SEQUENCE LISTING (76 sequences)

SEQ ID NO: 1 - DNA, 885 bp, Homo sapiens
  ref <308>: DATABASE ACCESSION NUMBER: NCBI/NM_181337
  ref <309>: DATABASE ENTRY DATE: 1999-12-20
  ref <313>: RELEVANT RESIDUES IN SEQ ID NO: (213)..(683)
gaggggcatcaatcacaccgagaagtcacagcccctcaaccactgaggtgtgggggggtagggatctgcatttcttcatatcaaccccacactatagggcacctaaatgggtgggcggtgggggagaccgactcacttgagtttcttgaaggcttcctggcctccagccacgtaattgcccccgctctggatctggtctagcttccggattcggtggccagtccgcggggtgtagatgttcctgacggccccaaagggtgcctgaacgccgccggtcacctccttcaggaagacttcgaagctggacaccttcttctcatggatgacgacgcggcgccccgcgtagaaggggtccccgttgcggtacacaagcacgctcttcacgacgggctgagacaggtggctggacctggcgctgctgccgctcatcttccccgctggccgccgcctcagctcgctgcttcgcgtcgggaggcacctccgctgtcccagcggcctcaccgcacccagggcgcgggatcgcctcctgaaacgaacgagaaactgacgaatccacaggtgaaagagaagtaacggccgtgcgcctaggcgtccacccagaggagacactaggagcttgcaggactcggagtagacgctcaagtttttcaccgtggcgtgcacagccaatcaggacccgcagtgcgcgcaccacaccaggttcacctgctacgggcagaatcaaggtggacagcttctgagcaggagccggaaacgcgcggggccttcaaacaggcacgcctagtgagggcaggagagaggaggacgcacacacacacacacacacaaatatggtgaaacccaatttcttacatcatatctgtgctaccctttccaaacagccta

SEQ ID NO: 2 - protein, 84 aa, Homo sapiens
  ref <308>: DATABASE ACCESSION NUMBER: NCBI/NP_851854.1
  ref <309>: DATABASE ENTRY DATE: 1999-12-20
  ref <313>: RELEVANT RESIDUES IN SEQ ID NO: (1)..(84)
MDDDAAPRVEGVPVAVHKHALHDGLRQVAGPGAAAAHLPRWPPPQLAASRREAPPLSQRPHRTQGAGSPPETNEKLTNPQVKEK

SEQ ID NO: 3 - DNA, 657 bp, Artificial Sequence
  complete light chain of 3D3 mAb
gacattgtgatgacccagtctccatcctccctggctgtgtcaataggacagaaggtcactatgaactgcaagtccagtcagagccttttaaatagtaactttcaaaagaactttttggcctggtaccagcagaaaccaggccagtctcctaaacttctgatatactttgcatccactcgggaatctagtatccctgatcgcttcataggcagtggatctgggacagatttcactcttaccatcagcagtgtgcaggctgaagacctggcagattacttctgtcagcaacattatagcactccgctcacgttcggtgctgggaccaagctggagctgaaagctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 219 aa, Artificial Sequence
  complete light chain of 3D3 mAb
DIVMTQSPSSLAVSIGQKVTMNCKSSQSLLNSNFQKNFLAWYQQKPGQSPKLLIYFASTRESSIPDRFIGSGSGTDFTLTISSVQAEDLADYFCQQHYSTPLTFGAGTKLELKAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1329 bp, Artificial Sequence
  complete heavy chain of 3D3 mAb
gaggttcagctgcagcagtctgtagctgagctggtgaggcctggggcttcagtgacgctgtcctgcaaggcttcgggctacatatttactgactatgagatacactgggtgaagcagactcctgtgcatggcctggaatggattggggttattgatcctgaaactggtaatactgccttcaatcagaagttcaagggcaaggccacactgactgcagacatatcctccagcacagcctacatggaactcagcagtttgacatctgaggactctgccgtctattactgtatgggttattctgattattggggccaaggcaccactctcacagtctcctcagcctcaacgaagggcccatctgtctttcccctggccccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgaattcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 6 - protein, 443 aa, Artificial Sequence
  complete heavy chain of 3D3 mAb
EVQLQQSVAELVRPGASVTLSCKASGYIFTDYEIHWVKQTPVHGLEWIGVIDPETGNTAFNQKFKGKATLTADISSSTAYMELSSLTSEDSAVYYCMGYSDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 654 bp, Artificial Sequence
  complete light chain of 3G10 mAb
gatgttttgatgacccaaactccacgctccctgtctgtcagtcttggagatcaagcctccatctcttgtagatcgagtcagagccttttacatagtaatggaaacacctatttagaatggtatttgcagaaaccaggccagcctccaaaggtcctgatctacaaagtttccaaccgattttctggggtcccagacaggttcagtggcagtggatcagggacagatttcacactcaagatcagcggagtggaggctgaggatctgggagtttattactgctttcaaggttcacatgttcctctcacgttcggtgctgggaccaagctggagctgaaagctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 218 aa, Artificial Sequence
  complete light chain of 3G10 mAb
DVLMTQTPRSLSVSLGDQASISCRSSQSLLHSNGNTYLEWYLQKPGQPPKVLIYKVSNRFSGVPDRFSGSGSGTDFTLKISGVEAEDLGVYYCFQGSHVPLTFGAGTKLELKAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1335 bp, Artificial Sequence
  complete heavy chain of 3G10 mAb
gagatccagctgcagcagtctggacctgagttggtgaagcctggggcttcagtgaagatatcctgtaaggcttctggatacaccttcactgacaactacatgaactgggtgaagcagagccatggaaagagccttgagtggattggagatattaatccttactatggtactactacctacaaccagaagttcaagggcaaggccacattgactgtagacaagtcctcccgcacagcctacatggagctccgcggcctgacatctgaggactctgcagtctattactgtgcaagagatgactggtttgattattggggccaagggactctggtcactgtctctgcagcctcaacgaagggcccatctgtctttcccctggccccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgaattcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 10 - protein, 445 aa, Artificial Sequence
  complete heavy chain of 3G10 mAb
EIQLQQSGPELVKPGASVKISCKASGYTFTDNYMNWVKQSHGKSLEWIGDINPYYGTTTYNQKFKGKATLTVDKSSRTAYMELRGLTSEDSAVYYCARDDWFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 639 bp, Artificial Sequence
  complete light chain of 3C4 mAb
gacatcgttatgtctcagtctccatcttccatgtatgcatctctaggagagagagtcactatcacttgcaaggcgagtcaggacattcataactttttaaactggttccagcagaaaccaggaaaatctccaaagaccctgatctttcgtgcaaacagattggtagatggggtcccatcaaggttcagtggcagtggatctgggcaagattattctctcaccatcagcagcctggagtttgaagatttgggaatttattcttgtctacagtatgatgagattccgctcacgttcggtgctgggaccaagctggagctgagagctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 12 - protein, 213 aa, Artificial Sequence
  complete light chain of 3C4 mAb
DIVMSQSPSSMYASLGERVTITCKASQDIHNFLNWFQQKPGKSPKTLIFRANRLVDGVPSRFSGSGSGQDYSLTISSLEFEDLGIYSCLQYDEIPLTFGAGTKLELRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1341 bp, Artificial Sequence
  complete heavy chain of 3C4 mAb
gaggtgcagcttcaggagtcaggacctgacctggtgaaaccttctcagtcactttcactcacctgcactgtcactggcttctccatcaccagtggttatggctggcactggatccggcagtttccaggaaacaaactggagtggatgggctacataaactacgatggtcacaatgactacaacccatctctcaaaagtcgaatctctatcactcaagacacatccaagaaccagttcttcctgcagttgaattctgtgactactgaggacacagccacatattactgtgcaagcagttacgacggcttatttgcttactggggccaagggactctggtcactgtctctgcagcctcaacgaagggcccatctgtctttcccctggccccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgaattcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 14 - protein, 447 aa, Artificial Sequence
  complete heavy chain of 3C4 mAb
EVQLQESGPDLVKPSQSLSLTCTVTGFSITSGYGWHWIRQFPGNKLEWMGYINYDGHNDYNPSLKSRISITQDTSKNQFFLQLNSVTTEDTATYYCASSYDGLFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 339 bp, Artificial Sequence
  light chain variable region of 3D3 mAb
gacattgtgatgacccagtctccatcctccctggctgtgtcaataggacagaaggtcactatgaactgcaagtccagtcagagccttttaaatagtaactttcaaaagaactttttggcctggtaccagcagaaaccaggccagtctcctaaacttctgatatactttgcatccactcgggaatctagtatccctgatcgcttcataggcagtggatctgggacagatttcactcttaccatcagcagtgtgcaggctgaagacctggcagattacttctgtcagcaacattatagcactccgctcacgttcggtgctgggaccaagctggagctgaaa

SEQ ID NO: 16 - protein, 113 aa, Artificial Sequence
  light chain variable region of 3D3 mAb
DIVMTQSPSSLAVSIGQKVTMNCKSSQSLLNSNFQKNFLAWYQQKPGQSPKLLIYFASTRESSIPDRFIGSGSGTDFTLTISSVQAEDLADYFCQQHYSTPLTFGAGTKLELK

SEQ ID NO: 17 - DNA, 339 bp, Artificial Sequence
  heavy chain variable region of 3D3 mAb
gaggttcagctgcagcagtctgtagctgagctggtgaggcctggggcttcagtgacgctgtcctgcaaggcttcgggctacatatttactgactatgagatacactgggtgaagcagactcctgtgcatggcctggaatggattggggttattgatcctgaaactggtaatactgccttcaatcagaagttcaagggcaaggccacactgactgcagacatatcctccagcacagcctacatggaactcagcagtttgacatctgaggactctgccgtctattactgtatgggttattctgattattggggccaaggcaccactctcacagtctcctca

SEQ ID NO: 18 - protein, 113 aa, Artificial Sequence
  heavy chain variable region of 3D3 mAb
EVQLQQSVAELVRPGASVTLSCKASGYIFTDYEIHWVKQTPVHGLEWIGVIDPETGNTAFNQKFKGKATLTADISSSTAYMELSSLTSEDSAVYYCMGYSDYWGQGTTLTVSS

SEQ ID NO: 19 - DNA, 336 bp, Artificial Sequence
  light chain variable region of 3G10 mAb
gatgttttgatgacccaaactccacgctccctgtctgtcagtcttggagatcaagcctccatctcttgtagatcgagtcagagccttttacatagtaatggaaacacctatttagaatggtatttgcagaaaccaggccagcctccaaaggtcctgatctacaaagtttccaaccgattttctggggtcccagacaggttcagtggcagtggatcagggacagatttcacactcaagatcagcggagtggaggctgaggatctgggagtttattactgctttcaaggttcacatgttcctctcacgttcggtgctgggaccaagctggagctgaaa

SEQ ID NO: 20 - protein, 112 aa, Artificial Sequence
  light chain variable region of 3G10 mAb
DVLMTQTPRSLSVSLGDQASISCRSSQSLLHSNGNTYLEWYLQKPGQPPKVLIYKVSNRFSGVPDRFSGSGSGTDFTLKISGVEAEDLGVYYCFQGSHVPLTFGAGTKLELK

SEQ ID NO: 21 - DNA, 345 bp, Artificial Sequence
  heavy chain variable region of 3G10 mAb
gagatccagctgcagcagtctggacctgagttggtgaagcctggggcttcagtgaagatatcctgtaaggcttctggatacaccttcactgacaactacatgaactgggtgaagcagagccatggaaagagccttgagtggattggagatattaatccttactatggtactactacctacaaccagaagttcaagggcaaggccacattgactgtagacaagtcctcccgcacagcctacatggagctccgcggcctgacatctgaggactctgcagtctattactgtgcaagagatgactggtttgattattggggccaagggactctggtcactgtctctgca

SEQ ID NO: 22 - protein, 115 aa, Artificial Sequence
  heavy chain variable region of 3G10 mAb
EIQLQQSGPELVKPGASVKISCKASGYTFTDNYMNWVKQSHGKSLEWIGDINPYYGTTTYNQKFKGKATLTVDKSSRTAYMELRGLTSEDSAVYYCARDDWFDYWGQGTLVTVSA

SEQ ID NO: 23 - DNA, 321 bp, Artificial Sequence
  light chain variable region of 3C4 mAb
gacatcgttatgtctcagtctccatcttccatgtatgcatctctaggagagagagtcactatcacttgcaaggcgagtcaggacattcataactttttaaactggttccagcagaaaccaggaaaatctccaaagaccctgatctttcgtgcaaacagattggtagatggggtcccatcaaggttcagtggcagtggatctgggcaagattattctctcaccatcagcagcctggagtttgaagatttgggaatttattcttgtctacagtatgatgagattccgctcacgttcggtgctgggaccaagctggagctgaga

SEQ ID NO: 24 - protein, 107 aa, Artificial Sequence
  light chain variable region of 3C4 mAb
DIVMSQSPSSMYASLGERVTITCKASQDIHNFLNWFQQKPGKSPKTLIFRANRLVDGVPSRFSGSGSGQDYSLTISSLEFEDLGIYSCLQYDEIPLTFGAGTKLELR

SEQ ID NO: 25 - DNA, 351 bp, Artificial Sequence
  heavy chain variable region of 3C4 mAb
gaggtgcagcttcaggagtcaggacctgacctggtgaaaccttctcagtcactttcactcacctgcactgtcactggcttctccatcaccagtggttatggctggcactggatccggcagtttccaggaaacaaactggagtggatgggctacataaactacgatggtcacaatgactacaacccatctctcaaaagtcgaatctctatcactcaagacacatccaagaaccagttcttcctgcagttgaattctgtgactactgaggacacagccacatattactgtgcaagcagttacgacggcttatttgcttactggggccaagggactctggtcactgtctctgca

SEQ ID NO: 26 - protein, 117 aa, Artificial Sequence
  heavy chain variable region of 3C4 mAb
EVQLQESGPDLVKPSQSLSLTCTVTGFSITSGYGWHWIRQFPGNKLEWMGYINYDGHNDYNPSLKSRISITQDTSKNQFFLQLNSVTTEDTATYYCASSYDGLFAYWGQGTLVTVSA

SEQ ID NO: 27 - DNA, 44 bp, Artificial Sequence
  Primer OGS364
  misc_feature (1)..(1): biotin
nactgtactaaccctgcggccgcttttttttttttttttttttv

SEQ ID NO: 28 - DNA, 47 bp, Artificial Sequence
  Primer OGS594
ggaattctaatacgactcactatagggagacgaagacagtagacagg

SEQ ID NO: 29 - DNA, 50 bp, Artificial Sequence
  Primer OGS595
cgcgcctgtctactgtcttcgtctccctatagtgagtcgtattagaattc

SEQ ID NO: 30 - DNA, 52 bp, Artificial Sequence
  Primer OGS458
ggaattctaatacgactcactatagggagagcctgcaccaacagttaacagg

SEQ ID NO: 31 - DNA, 55 bp, Artificial Sequence
  Primer OGS459
cgcgcctgttaactgttggtgcaggctctccctatagtgagtcgtattagaattc

SEQ ID NO: 32 - DNA, 19 bp, Artificial Sequence
  Primer OGS494
gggagacgaagacagtaga

SEQ ID NO: 33 - DNA, 20 bp, Artificial Sequence
  Primer OGS302
gcctgcaccaacagttaaca

SEQ ID NO: 34 - DNA, 28 bp, Artificial Sequence
  Primer OGS621
ggaattctaatacgactcactataggga

SEQ ID NO: 35 - DNA, 31 bp, Artificial Sequence
  Primer OGS622
cgcgtccctatagtgagtcgtattagaattc

SEQ ID NO: 36 - DNA, 2757 bp, Artificial Sequence
  plasmid PCATRMAN
ttttcccagtcacgacgttgtaaaacgacggccagtgaattctaatacgactcactatagggagatggagaaaaaaatcactggacgcgtggcgcgccattaattaatgcggccgctagctcgagtgataataagcggatgaatggctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccaggg

SEQ ID NO: 37 - DNA, 2995 bp, Artificial Sequence
  plasmid p20
ttttcccagtcacgacgttgtaaaacgacggccagtgaattcaattaaccctcactaaagggagacttgttccaaatgtgttaggcgcgccgcatgcgtcgacggatcctgagaacttcaggctcctgggcaacgtgctggttattgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaaaaaaaaaaagcggccgctcttctatagtgtcacctaaatggcccagcggccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccaggg

SEQ ID NO: 38 - DNA, 26 bp, Artificial Sequence
  Primer OGS315
tgaaggtcggagtcaacggatttggt

SEQ ID NO: 39 - DNA, 24 bp, Artificial Sequence
  Primer OGS316
catgtgggccatgaggtccaccac

SEQ ID NO: 40 - DNA, 24 bp, Artificial Sequence
  KAAG1 primer
gaggggcatcaatcacaccgagaa

SEQ ID NO: 41 - DNA, 22 bp, Artificial Sequence
  KAAG1 primer
ccccaccgcccacccatttagg

SEQ ID NO: 42 - DNA, 26 bp, Artificial Sequence
  GAPDH primer
tgaaggtcggagtcaacggatttggt

SEQ ID NO: 43 - DNA, 24 bp, Artificial Sequence
  GAPDH primer
catgtgggccatgaggtccaccac

SEQ ID NO: 44 - DNA, 19 bp, Artificial Sequence
  siRNA
ggcctccagccacgtaatt

SEQ ID NO: 45 - DNA, 19 bp, Artificial Sequence
  siRNA
ggcgctgctgccgctcatc

SEQ ID NO: 46 - DNA, 4455 bp, Artificial Sequence
  Primer OGS1068
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttttccaaaaaactaccgttgttataggtgtctcttgaacacctataacaacggtagtggatcccgcgtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggcgcgcgctcggcgcgcggtcacgctccgtcacgtggtgcgttttgcctgcgcgtctttccactggggaattcatgcttctcctccctttagtgagggtaattctctctctctccctatagtgagtcgtattaattccttctcttctatagtgtcacctaaatcgttgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaaaaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctattggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccacccggggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcaatctaagaaaccattattatcatgacattaacctataaaaataggcgtatcacgaggccctttcgtc

SEQ ID NO: 47 - DNA, 33 bp, Artificial Sequence
  Primer
gtaagcggatccatggatgacgacgcggcgccc

SEQ ID NO: 48 - DNA, 33 bp, Artificial Sequence
  Primer
gtaagcaagcttcttctctttcacctgtggatt

SEQ ID NO: 49 - DNA, 5138 bp, Artificial Sequence
  expression vector pYD5
gtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagccagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagccggcggatggtcgaggtgaggtgtggcaggcttgagatccagctgttggggtgagtactccctctcaaaagcgggcatgacttctgcgctaagattgtcagtttccaaaaacgaggaggatttgatattcacctggcccgatctggccatacacttgagtgacaatgacatccactttgcctttctctccacaggtgtccactcccaggtccaagtttgccgccaccatggagacagacacactcctgctatgggtactgctgctctgggttccaggttccactggcgccggatcaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaagctagcggagccggaagcacaaccgaaaacctgtattttcagggcggatccgaattcaagcttgatatctgatcccccgacctcgacctctggctaataaaggaaatttattttcattgcaatagtgtgttggaattttttgtgtctctcactcggaaggacatatgggagggcaaatcatttggtcgagatccctcggagatctctagctagagccccgccgccggacgaactaaacctgactacggcatctctgccccttcttcgcggggcagtgcatgtaatcccttcagttggttggtacaacttgccaactgaaccctaaacgggtagcatatgcttcccgggtagtagtatatactatccagactaaccctaattcaatagcatatgttacccaacgggaagcatatgctatcgaattagggttagtaaaagggtcctaaggaacagcgatgtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcacgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 50 - DNA, 36 bp, Artificial Sequence
  Primer
gtaagcaagcttaggccgctgggacagcggaggtgc

SEQ ID NO: 51 - DNA, 33 bp, Artificial Sequence
  Primer
gtaagcaagcttggcagcagcgccaggtccagc

SEQ ID NO: 52 - DNA, 33 bp, Artificial Sequence
  Primer OGS1773
gtaagcagcgctgtggctgcaccatctgtcttc

SEQ ID NO: 53 - DNA, 35 bp, Artificial Sequence
  Primer OGS1774
gtaagcgctagcctaacactctcccctgttgaagc

SEQ ID NO: 54 - DNA, 321 bp, Artificial Sequence
  human kappa constant region
gctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 55 - protein, 106 aa, Artificial Sequence
  human kappa constant region
AVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - DNA, 6385 bp, Artificial Sequence
  plasmid pTTVK1
cttgagccggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgaattcatgaactttctgctgtcttgggtgcattggagccttgccttgctgctctacctccaccatgccaagtggtcccaggcttgagacggagcttacagcgctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggtaccgcggccgcttcgaatgagatcccccgacctcgacctctggctaataaaggaaatttattttcattgcaatagtgtgttggaattttttgtgtctctcactcggaaggacatatgggagggcaaatcatttggtcgagatccctcggagatctctagctagagccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcacgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgcattgctgcaggcgcagaactggtaggtatggcagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggt

SEQ ID NO: 57 - DNA, 43 bp, Artificial Sequence
  Primer
atgccaagtggtcccaggctgacattgtgatgacccagtctcc

SEQ ID NO: 58 - DNA, 43 bp, Artificial Sequence
  Primer
atgccaagtggtcccaggctgatgttttgatgacccaaactcc

SEQ ID NO: 59 - DNA, 43 bp, Artificial Sequence
  Primer
atgccaagtggtcccaggctgacatcgttatgtctcagtctcc

SEQ ID NO: 60 - DNA, 32 bp, Artificial Sequence
  Primer
gggaagatgaagacagatggtgcagccacagc

SEQ ID NO: 61 - DNA, 50 bp, Artificial Sequence
  Primer OGS1769
gtaagcgctagcgcctcaacgaagggcccatctgtctttcccctggcccc

SEQ ID NO: 62 - DNA, 37 bp, Artificial Sequence
  Primer OGS1770
gtaagcgaattcacaagatttgggctcaactttcttg

SEQ ID NO: 63 - DNA, 309 bp, Artificial Sequence
  human IgG1 CH1 region
gcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 64 - protein, 103 aa, Artificial Sequence
  human IgG1 CH1 region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 65 - DNA, 5379 bp, Artificial Sequence
  Plasmid pYD15
cttgagccggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttgccgccaccatggagacagacacactcctgctatgggtactgctgctctgggttccaggttccactggcggagacggagcttacgggcccatctgtctttcccctggccccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgaattcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgatcccccgacctcgacctctggctaataaaggaaatttattttcattgcaatagtgtgttggaattttttgtgtctctcactcggaaggacatatgggagggcaaatcatttggtcgagatccctcggagatctctagctagagccccgccgccggacgaactaaacctgactacggcatctctgccccttcttcgcggggcagtgcatgtaatcccttcagttggttggtacaacttgccaactgaaccctaaacgggtagcatatgcttcccgggtagtagtatatactatccagactaaccctaattcaatagcatatgttacccaacgggaagcatatgctatcgaattagggttagtaaaagggtcctaaggaacagcgatgtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcacgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggt

SEQ ID NO: 66 - DNA, 43 bp, Artificial Sequence
  Primer
gggttccaggttccactggcgaggttcagctgcagcagtctgt

SEQ ID NO: 67 - DNA, 43 bp, Artificial Sequence
  Primer
gggttccaggttccactggcgaggtgcagcttcaggagtcagg

SEQ ID NO: 68 - DNA, 38 bp, Artificial Sequence
  Primer
ggggccaggggaaagacagatgggcccttcgttgaggc

SEQ ID NO: 69 - protein, 240 aa, Artificial Sequence
  complete humanized 3D3 light chain
MVLQTQVFISLLLWISGAYGDIVMTQSPDSLAVSLGERATINCKSSQSLLNSNFQKNFLAWYQQKPGQPPKLLIYFASTRESSVPDRFSGSGSGTDFTLTISSLQAEDVAVYYCQQHYSTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 462 aa, Artificial Sequence
  complete humanized 3D3 heavy chain
MDWTWRILFLVAAATGTHAEVQLVQSGAEVKKPGASVKVSCKASGYIFTDYEIHWVRQAPGQGLEWMGVIDPETGNTAFNQKFKGRVTITADTSTSTAYMELSSLTSEDTAVYYCMGYSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 113 aa, Artificial Sequence
  variable region of the humanized 3D3 light chain
DIVMTQSPDSLAVSLGERATINCKSSQSLLNSNFQKNFLAWYQQKPGQPPKLLIYFASTRESSVPDRFSGSGSGTDFTLTISSLQAEDVAVYYCQQHYSTPLTFGQGTKLEIK

SEQ ID NO: 72 - protein, 113 aa, Artificial Sequence
  variable region of the humanized 3D3 heavy chain
EVQLVQSGAEVKKPGASVKVSCKASGYIFTDYEIHWVRQAPGQGLEWMGVIDPETGNTAFNQKFKGRVTITADTSTSTAYMELSSLTSEDTAVYYCMGYSDYWGQGTLVTVSS

SEQ ID NO: 73 - protein, 234 aa, Artificial Sequence
  complete humanized 3C4 light chain
MVLQTQVFISLLLWISGAYGDIVMTQSPSSLSASVGDRVTITCKASQDIHNFLNWFQQKPGKAPKTLIFRANRLVDGVPSRFSGSGSGTDYTLTISSLQPEDFATYSCLQYDEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 466 aa, Artificial Sequence
  complete humanized 3C4 heavy chain
MDWTWRILFLVAAATGTHAEVQLQESGPGLVKPSQTLSLTCTVSGFSITSGYGWHWIRQHPGKGLEWIGYINYDGHNDYNPSLKSRVTISQDTSKNQFSLKLSSVTAADTAVYYCASSYDGLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 107 aa, Artificial Sequence
  variable region of the humanized 3C4 light chain
DIVMTQSPSSLSASVGDRVTITCKASQDIHNFLNWFQQKPGKAPKTLIFRANRLVDGVPSRFSGSGSGTDYTLTISSLQPEDFATYSCLQYDEIPLTFGQGTKLEIK

SEQ ID NO: 76 - protein, 116 aa, Artificial Sequence
  variable region of the humanized 3C4 heavy chain
EVQLQESGPGLVKPSQTLSLTCTVSGFSITSGYGWHWIRQHPGKGLEWIGYINYDGHNDYNPSLKSRVTISQDTSKNQFSLKLSSVTAADTAVYYCASSYDGLFAYWGQGTLVTVS

(19) United States
(12) Patent Application Publication
(10) Pub. No.: US 2012/0128661 A1
(43) Pub. Date: May 24, 2012

(54) POLYNUCLEOTIDE AND POLYPEPTIDE SEQUENCES INVOLVED IN CANCER

(75) Inventors: Roy Rabindranauth Sooknanan, Beaconsfield (CA); Gilles Bernard Tremblay, La Prairie (CA); Mario Filion, Longueuil (CA)

(73) Assignee: ALETHIA BIOTHERAPEUTICS INC., Montreal (CA)

(21) Appl. No.: 13/152,209

(22) Filed: Jun. 2, 2011

Related U.S. Application Data
(63) Continuation-in-part of application No. 12/305,648, filed on Nov. 6, 2009.
(60) Provisional application No. 60/815,829, filed on Jun. 23, 2006.
(60) Provisional application No. 60/874,471, filed on Dec. 13, 2006.

(51) Int. Cl. A61K 39/395; A61P 35/00

(52) U.S. Cl. 424/133.1; 424/139.1; 424/178.1

(57) ABSTRACT

The present invention relates to polynucleotide and polypeptide sequences which are differentially expressed in cancer cells compared to normal cells. The present invention more particularly relates to the use of these sequences in the diagnosis, prognosis or treatment of cancer and in the detection of cancer cells.

The present application is a continuation-in-part of U.S. Ser. No. 12/305,648 filed on Jun. 22, 2007, the entire content of which is incorporated herein by reference, which application claims the benefit of U.S. Provisional application Ser. No. 60/815,829 filed on Jun. 23, 2006 and U.S. Provisional application Ser. No. 60/874,471 filed on Dec. 13, 2006, the entire content of which is incorporated herein by reference.

### FIELD OF THE INVENTION

The present invention relates to polynucleotide and polypeptide sequences which are differentially expressed in cancer compared to normal cells. The present invention more particularly relates to the use of these sequences in the diagnosis, prognosis or treatment of cancer and in the detection of cancer cells.

### BACKGROUND OF THE INVENTION

Among gynecologic malignancies, ovarian cancer accounts for the highest tumor-related mortality in women in the United States (Jemal et al., 2005). It is the fourth leading cause of cancer-related death in women in the U.S (Menon et al., 2005). The American Cancer Society estimated a total of 22,220 new cases in 2005 and attributed 16,210 deaths to the disease (Bonome et al., 2005). For the past 30 years, the statistics have remained largely the same—the majority of women who develop ovarian cancer will die of this disease (Chambers and Vanderhyden, 2006). The disease carries a 1:70 lifetime risk and a mortality rate of >60% (Chambers and Vanderhyden, 2006). The high mortality rate is due to the difficulties with the early detection of ovarian cancer when the malignancy has already spread beyond the ovary. Indeed, >80% of patients are diagnosed with advanced staged disease (stage III or IV) (Bonome et al., 2005). These patients have a poor prognosis that is reflected in <45% 5-year survival rate, although 80% to 90% will initially respond to chemotherapy (Berek et al., 2000). This increased success compared to 20% 5-year survival rate years earlier is, at least in part, due to the ability to optimally debulk tumor tissue when it is confined to the ovaries, which is a significant prognostic factor for ovarian cancer (Bristow R. E., 2000 and Brown et al., 2004). In patients who are diagnosed with early disease (stage I), the 5-yr survival ranges from >90 (Chambers and Vanderhyden, 2006).

Ovarian cancer comprises a heterogeneous group of tumors that are derived from the surface epithelium of the ovary or from surface inclusions. They are classified into serous, mucinous, endometrioid, clear cell, and Brenner (transitional) types corresponding to the different types of epithelia in the organs of the female reproductive tract (Shih and Kurman, 2005). Of these, serous tumors account for ˜60% of the ovarian cancer cases diagnosed. Each histologic subcategory is further divided into three groups: benign, intermediate (borderline tumor or low malignancy potential (LMP)), and malignant, reflecting their clinical behavior (Seidman et al., 2002). LMP represents 10% to 15% of tumors diagnosed as serous and is a conundrum as they display atypical nuclear structure and metastatic behavior, yet they are considerably less aggressive than high-grade serous tumors. The 5-year survival for patients with LMP tumors is 95% in contrast to a <45% survival for advanced high-grade disease over the same period (Berek et al., 2000).

Despite improved knowledge of the etiology of the disease, aggressive cytoreductive surgery, and modern combination chemotherapy, there has been only little change in mortality. Poor outcomes have been attributed to (1) lack of adequate screening tests for early disease detection, in combination with only subtle presentation of symptoms at this stage—diagnosis is frequently being made only after progression to later stages, at which point the peritoneal dissemination of the cancer limits effective treatment and (2) the frequent development of resistance to standard chemotherapeutic strategies limiting improvement in the 5-year survival rate of patients. The initial chemotherapy regimen for ovarian cancer includes the combination of carboplatin (Paraplatin) and paclitaxel (taxol). Years of clinical trials have proved this combination to be most effective after effective surgery—reduces tumor volume in about 80% of the women with newly diagnosed ovarian cancer and 40% to 50% will have complete regression—but studies continue to look for ways to improve it. Recent abdominal infusion of chemotherapeutics to target hard-to-reach cells in combination with intravenous delivery has increased the effectiveness. However, severe side effects often lead to an incomplete course of treatment. Some other chemotherapeutic agents include doxorubicin, cisplatin, cyclophosphamide, bleomycin, etoposide, vinblastine, topotecan hydrochloride, ifosfamide, 5-fluorouracil and melphalan. The excellent survival rates for women with early stage disease receiving chemotherapy provide a strong rationale for research efforts to develop strategies to improve the detection of ovarian cancer. Furthermore, the discovery of new ovarian cancer-related biomarkers will lead to the development of more effective therapeutic strategies with minimal side effects for the future treatment of ovarian cancer.

Presently, the diagnosis of ovarian cancer is accomplished, in part, through routine analysis of the medical history of patients and by performing physical, ultrasound and x-ray examinations, and hematological screening. Two alternative strategies have been reported for early hematological detection of serum biomarkers. One approach is the analysis of serum samples by mass spectrometry to find proteins or protein fragments of unknown identity that detect the presence or absence of cancer (Mor et al., 2005 and Kozak et al., 2003). However, this strategy is expensive and not broadly available. Alternatively, the presence or absence of known proteins/peptides in the serum is being detected using antibody microarrays, ELISA, or other similar approaches. Serum testing for a protein biomarker called CA-125 (cancer antigen-125) has long been widely performed as a marker for ovarian cancer. However, although ovarian cancer cells may produce an excess of these protein molecules, there are some other cancers, including cancer of the fallopian tube or endometrial cancer (cancer of the lining of the uterus), 60% of people with pancreatic cancer, and 20%-25% of people with other malignancies with elevated levels of CA-125. The CA-125 test only returns a true positive result for about 50% of Stage I ovarian cancer patients and has a80% chance of returning true positive results from stage II, III, and IV ovarian cancer patients. The other 20% of ovarian cancer patients do not show any increase in CA-125 concentrations. In addition, an elevated CA-125 test may indicate other benign activity not associated with cancer, such as menstruation, pregnancy, or endometriosis. Consequently, this test has very limited clinical application for the detection of early stage disease when it is still treatable, exhibiting a positive predictive value (PPV) of <10%. And, even with the addition of ultrasound screening to CA-125, the PPV only improves to around 20% (Kozak et al., 2003). Thus, this test is not an effective screening test.

Other studies have yielded a number of biomarker combinations with increased specificity and sensitivity for ovarian cancer relative to CA-125 alone (McIntosh et al., 2004, Woolas et al., 1993, Schorge et., 2004). Serum biomarkers that are often elevated in women with epithelial ovarian cancer, but not exclusively, include carcinoembryonic antigen, ovarian cystadenocarcinoma antigen, lipidassociated sialic acid, NB/70, TAG72.3, CA-15.3, and CA-125. Unfortunately, although this approach has increased the sensitivity and specificity of early detection, published biomarker combinations still fail to detect a significant percentage of stage I/II epithelial ovarian cancer. Another study (Elieser et al., 2005) measured serum concentrations of 46 biomarkers including CA-125 and amongst these, 20 proteins in combination correctly recognized more than 98% of serum samples of women with ovarian cancer compared to other benign pelvic disease. Although other malignancies were not included in this study, this multimarker panel assay provided the highest diagnostic power for early detection of ovarian cancer thus far.

Additionally, with the advent of differential gene expression analysis technologies, for example DNA microarrays and subtraction methods, many groups have now reported large collections of genes that are upregulated in epithelial ovarian cancer (United States patent application published under numbers; 20030124579, 20030087250, 20060014686, 20060078941, 20050095592, 20050214831, 20030219760, 20060078941, 20050214826). However, the clinical utilities with respect to ovarian cancer of one or combinations of these genes are not as yet fully determined.

There is a need for new tumor biomarkers for improving diagnosis and/or prognosis of cancer. In addition, due to the genetic diversity of tumors, and the development of chemoresistance by many patients, there exists further need for better and more universal therapeutic approaches for the treatment of cancer. Molecular targets for the development of such therapeutics may preferably show a high degree of specificity for the tumor tissues compared to other somatic tissues, which will serve to minimize or eliminate undesired side effects, and increase the efficacy of the therapeutic candidates.

This present invention tries to address these needs and other needs.

### SUMMARY OF THE INVENTION

In accordance with the present invention, there is provided new polynucleotide sequences and new polypeptide sequences as well as compositions, antibodies specific for these sequences, vectors and cells comprising a recombinant form of these new sequences.

The present invention also provides methods of detecting cancer cells using single or multiple polynucleotides and/or polypeptide sequences which are specific to these tumor cells. Some of the polynucleotides and/or polypeptides sequences provided herein are differentially expressed in ovarian cancer compared to normal cells and may also be used to distinguish between malignant ovarian cancer and an ovarian cancer of a low malignancy potential and/or a normal state (individual free of ovarian cancer).

Also encompassed by the present invention are diagnostic methods, prognostic methods, methods of detection, kits, arrays, libraries and assays which comprises one or more polypeptide and/or polynucleotide sequences or antibodies described herein as well as new therapeutic avenues for cancer treatment.

The Applicant has come to the surprising discovery that polynucleotide and/or polypeptide sequences described herein are preferentially upregulated in malignant ovarian cancer compared to low malignancy potential ovarian cancer and/or compared to normal cells. More interestingly, some of these sequences appear to be overexpressed in late stage ovarian cancer.

The Applicant has also come to the surprising discovery that some of the sequences described herein are not only expressed in ovarian cancer cells but in other cancer cells such as cells from breast cancer, prostate cancer, renal cancer, colon cancer, lung cancer, melanoma, leukemia and from cancer of the central nervous system. As such, several of these sequences, either alone or in combination may represent universal tumor markers. Therefore, some NSEQs and PSEQs described herein not only find utility in the field of ovarian cancer detection and treatment but also in the detection and treatment of other types of tumors

Therefore, using NSEQs or PSEQs of the present invention, one may readily identify a cell as being cancerous. As such NSEQs or PSEQs may be used to identify a cell as being a ovarian cancer cell, a prostate cancer cell, a breast cancer cell, a lung cancer cell, a colon cancer cell, a renal cancer cell, a cell from a melanoma, a leukemia cell or a cell from a cancer of the central nervous system.

Even more particularly, NSEQs or PSEQs described herein may be used to identify a cell as being a malignant ovarian cancer or a low malignant potential ovarian cancer.

The presence of some NSEQs or PSEQs in ovarian cancer cell may preferentially be indicative that the ovarian cancer is of the malignant type. Some NSEQs or PSEQs of the present invention may also more particularly indicate that the cancer is a late-stage malignant ovarian cancer.

The NSEQs or PSEQs may further be used to treat cancer or to identify compounds useful in the treatment of cancer including, ovarian cancer (i.e., LMP and/or malignant ovarian cancer), prostate cancer, breast cancer, lung cancer, colon cancer, renal cancer, melanoma, leukemia or cancer of the central nervous system.

As used herein and in some embodiments of the invention, the term “NSEQ” refers generally to polynucleotides sequences comprising or consisting of SEQ ID NO.:1 (KAAG1 nucleic acid sequence) (e.g., an isolated form) or comprising or consisting of a fragment of SEQ ID NO.:1. The term “NSEQ” more particularly refers to a polynucleotide sequence comprising or consisting of a transcribed portion of SEQ ID NO.:1, which may be, for example, free of untranslated or untranslatable portion(s) (i.e., a coding portion of SEQ ID NO.:1). The term “NSEQ” additionally refers to a sequence substantially identical to any one of the above and more particularly substantially identical to polynucleotide sequence comprising or consisting of a transcribed portion of SEQ ID NO.:1, which may be, for example, free of untranslated or untranslatable portion(s). The term “NSEQ” additionally refers to a nucleic acid sequence region of SEQ ID NO.:1 which encodes or is able to encode a polypeptide. The term “NSEQ” also refers to a polynucleotide sequence able to encode any one of the polypeptides described herein or a polypeptide fragment of any one of the above. Finally, the term “NSEQ” refers to a sequence substantially complementary to any one of the above.

As such, in embodiments of the invention NSEQ encompasses, for example, SEQ ID NO.:1 and also encompasses polynucleotide sequences which comprises, are designed or derived from SEQ ID NO.:1. Non-limiting examples of such sequences includes, for example, SEQ ID NOs.: 44 and 45.

The term “inhibitory NSEQ” generally refers to a sequence substantially complementary to SEQ ID NO.:1, substantially complementary to a fragment of SEQ ID NO.:1, substantially complementary to a sequence substantially identical to SEQ ID NO.:1 and more particularly, substantially complementary to a transcribed portion of SEQ ID NO.:1 (e.g., which may be free of untranslated or untranslatable portion) and which may have attenuating or even inhibitory action against the transcription of a mRNA or against expression of a polypeptide encoded by a corresponding SEQ ID NO.:1. Suitable “inhibitory NSEQ” may have for example and without limitation from about 10 to about 30 nucleotides, from about 10 to about 25 nucleotides or from about 15 to about 20 nucleotides.

As used herein the term “PSEQ” refers generally to each and every polypeptide sequences mentioned herein such as, for example, any polypeptide sequences encoded (putatively encoded) by any one of NSEQ described herein (e.g., any one of SEQ ID NO.:1) including their isolated or substantially purified form. Therefore, in embodiments of the invention, a polypeptide comprising or consisting SEQ ID NO.:2 including variants (e.g., an isolated natural protein variant), analogs, derivatives and fragments thereof are collectively referred to herein as “PSEQ”. Some of the NSEQs or PSEQs described herein have been previously characterized for purposes other than those described herein. As such diagnostics and therapeutics which are known to target those NSEQs or PSEQs (e.g., antibodies and/or inhibitors) may thus now be applied for inhibition of these NSEQs or PSEQs in the context of treatment of ovarian cancer, prostate cancer, renal cancer, colon cancer, lung cancer, melanoma, leukemia or cancer of the central nervous system. The use of these known therapeutics and diagnostics for previously undisclosed utility such as those described herein is encompassed by the present invention.

### Non-Limitative Exemplary Embodiments of the Invention

### Use of NSEQ as a Screening Tool

The NSEQ described herein may be used either directly or in the development of tools for the detection and isolation of expression products (mRNA, mRNA precursor, hnRNA, etc.), of genomic DNA or of synthetic products (cDNA, PCR fragments, vectors comprising NSEQ etc.). NSEQs may also be used to prepare suitable tools for detecting an encoded polypeptide or protein. NSEQ may thus be used to provide an encoded polypeptide and to generate an antibody specific for the polypeptide.

Those skilled in the art will also recognize that short oligonucleotides sequences may be prepared based on the polynucleotide sequences described herein. For example, oligonucleotides having 10 to 20 nucleotides or more may be prepared for specifically hybridizing to a NSEQ having a substantially complementary sequence and to allow detection, identification and isolation of nucleic sequences by hybridization. Probe sequences of for example, at least 10-20 nucleotides may be prepared based on a sequence found in SEQ ID NO.:1 and more particularly selected from regions that lack homology to undesirable sequences. Probe sequences of 20 or more nucleotides that lack such homology may show an increased specificity toward the target sequence. Useful hybridization conditions for probes and primers are readily determinable by those of skill in the art. Stringent hybridization conditions encompassed herewith are those that may allow hybridization of nucleic acids that are greater than 90% homologous but which may prevent hybridization of nucleic acids that are less than 70% homologous. The specificity of a probe may be determined by whether it is made from a unique region, a regulatory region, or from a conserved motif. Both probe specificity and the stringency of diagnostic hybridization or amplification (maximal, high, intermediate, or low) reactions depend on whether or not the probe identifies exactly complementary sequences, allelic variants, or related sequences. Probes designed to detect related sequences may have, for example, at least 50% sequence identity to any of the selected polynucleotides.

Furthermore, a probe may be labelled by any procedure known in the art, for example by incorporation of nucleotides linked to a “reporter molecule”. A “reporter molecule”, as used herein, may be a molecule that provides an analytically identifiable signal allowing detection of a hybridized probe. Detection may be either qualitative or quantitative. Commonly used reporter molecules include fluorophores, enzymes, biotin, chemiluminescent molecules, bioluminescent molecules, digoxigenin, avidin, streptavidin or radioisotopes. Commonly used enzymes include horseradish peroxidase, alkaline phosphatase, glucose oxidase and β-galactosidase, among others. Enzymes may be conjugated to avidin or streptavidin for use with a biotinylated probe. Similarly, probes may be conjugated to avidin or streptavidin for use with a biotinylated enzyme. Incorporation of a reporter molecule into a DNA probe may be effected by any method known to the skilled artisan, for example by nick translation, primer extension, random oligo priming, by 3′ or 5′ end labeling or by other means. In addition, hybridization probes include the cloning of nucleic acid sequences into vectors for the production of mRNA probes. Such vectors are known in the art, are commercially available, and may be used to synthesize RNA probes in vitro. The labelled polynucleotide sequences may be used in Southern or northern analysis, dot blot, or other membrane-based technologies; in PCR technologies; and in micro arrays utilizing samples from subjects to detect altered expression. Oligonucleotides useful as probes for screening of samples by hybridization assays or as primers for amplification may be packaged into kits. Such kits may contain the probes or primers in a pre-measured or predetermined amount, as well as other suitably packaged reagents and materials needed for the particular hybridization or amplification protocol.

The expression of mRNAs identical or substantially identical to the NSEQs of the present invention may thus be detected and/or isolated using methods that are known in the art. Exemplary embodiment of such methods includes, for example and without limitation, hybridization analysis using oligonucleotide probes, reverse transcription and in vitro nucleic acid amplification methods.

Such procedures may therefore, permit detection of mRNAs in ovarian cells (e.g., ovarian cancer cells) or in any other cells expressing such mRNAs. Expression of mRNA in a tissue-specific or a disease-specific manner may be useful for defining the tissues and/or particular disease state. One of skill in the art may readily adapt the NSEQs for these purposes.

It is to be understood herein that the NSEQs may hybridize to a substantially complementary sequence found in a test sample (e.g., cell, tissue, etc.). Additionally, a sequence substantially complementary to NSEQ (including fragments) may bind a NSEQ and substantially identical sequences found in a test sample (e.g., cell, tissue, etc.). Polypeptide encoded by an isolated NSEQ, polypeptide variants, polypeptide analogs or polypeptide fragments thereof are also encompassed herewith. The polypeptides whether in a premature, mature or fused form, may be isolated from lysed cells, or from the culture medium, and purified to the extent needed for the intended use. One of skill in the art may readily purify these proteins, polypeptides and peptides by any available procedure. For example, purification may be accomplished by salt fractionation, size exclusion chromatography, ion exchange chromatography, reverse phase chromatography, affinity chromatography and the like. Alternatively, PSEQ may be made by chemical synthesis.

Natural variants may be identified through hybridization screening of a nucleic acid library or polypeptide library from different tissue, cell type, population, species, etc using the NSEQ and derived tools.

### Use of NSEQ for Development of an Expression System

In order to express a polypeptide, a NSEQ able to encode any one of a PSEQ described herein may be inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

A variety of expression vector/host cell systems known to those of skill in the art may be utilized to express a polypeptide or RNA from NSEQ. These include, but are not limited to, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems. For long-term production of recombinant proteins in mammalian systems, stable expression in cell lines may be effected. For example, NSEQ may be transformed into cell lines using expression vectors that may contain viral origins of replication and/or endogenous expression elements and a selectable or visible marker gene on the same or on a separate vector. The invention is not to be limited by the vector or host cell employed.

Alternatively, RNA and/or polypeptide may be expressed from a vector comprising NSEQ using an in vitro transcription system or a coupled in vitro transcription/translation system respectively.

In general, host cells that contain NSEQ and/or that express a polypeptide encoded by the NSEQ, or a portion thereof, may be identified by a variety of procedures known to those of skill in the art. These procedures include, but are not limited to, DNA/DNA or DNA/RNA hybridizations, PCR amplification, and protein bioassay or immunoassay techniques that include membrane, solution, or chip based technologies for the detection and/or quantification of nucleic acid or amino acid sequences. Immunological methods for detecting and measuring the expression of polypeptides using either specific polyclonal or monoclonal antibodies are known in the art. Examples of such techniques include enzyme-linked immunosorbent assays (ELISAs), radioimmunoassays (RIAs), and fluorescence activated cell sorting (FACS). Those of skill in the art may readily adapt these methodologies to the present invention.

Host cells comprising NSEQ may thus be cultured under conditions for the transcription of the corresponding RNA (mRNA, siRNA, shRNA etc.) and/or the expression of the polypeptide from cell culture. The polypeptide produced by a cell may be secreted or may be retained intracellularly depending on the sequence and/or the vector used. As will be understood by those of skill in the art, expression vectors containing NSEQ may be designed to contain signal sequences that direct secretion of the polypeptide through a prokaryotic or eukaryotic cell membrane. Due to the inherent degeneracy of the genetic code, other DNA sequences that encode the same, substantially the same or a functionally equivalent amino acid sequence may be produced and used, for example, to express a polypeptide encoded by NSEQ. The nucleotide sequences of the present invention may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. In addition, a host cell strain may be chosen for its ability to modulate expression of the inserted sequences or to process the expressed polypeptide in the desired fashion. Such modifications of the polypeptide include, but are not limited to, acetylation, carboxylation, glycosylation, phosphorylation, lipidation, and acylation. Post-translational processing, which cleaves a “prepro” form of the polypeptide, may also be used to specify protein targeting, folding, and/or activity. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Those of skill in the art will readily appreciate that natural, modified, or recombinant nucleic acid sequences may be ligated to a heterologous sequence resulting in translation of a fusion polypeptide containing heterologous polypeptide moieties in any of the aforementioned host systems. Such heterologous polypeptide moieties may facilitate purification of fusion polypeptides using commercially available affinity matrices. Such moieties include, but are not limited to, glutathione S-transferase (GST), maltose binding protein, thioredoxin, calmodulin binding peptide, 6-His (His), FLAG, c-myc, hemaglutinin (HA), and antibody epitopes such as monoclonal antibody epitopes.

In yet a further aspect, the present invention relates to a polynucleotide which may comprise a nucleotide sequence encoding a fusion protein, the fusion protein may comprise a fusion partner fused to a peptide fragment of a protein encoded by, or a naturally occurring allelic variant polypeptide encoded by, the polynucleotide sequence described herein.

Those of skill in the art will also readily recognize that the nucleic acid and polypeptide sequences may be synthesized, in whole or in part, using chemical or enzymatic methods well known in the art. For example, peptide synthesis may be performed using various solid-phase techniques and machines such as the ABI 431A Peptide synthesizer (PE Biosystems) may be used to automate synthesis. If desired, the amino acid sequence may be altered during synthesis and/or combined with sequences from other proteins to produce a variant protein.

The present invention additionally relates to a bioassay for evaluating compounds as potential antagonists of the polypeptide described herein, the bioassay may comprise:
a) culturing test cells in culture medium containing increasing concentrations of at least one compound whose ability to inhibit the action of a polypeptide described herein is sought to be determined, wherein the test cells may contain a polynucleotide sequence described herein (for example, in a form having improved trans-activation transcription activity, relative to wild-type polynucleotide, and comprising a response element operatively linked to a reporter gene); and thereafter
b) monitoring in the cells the level of expression of the product of the reporter gene (encoding a reporter molecule) as a function of the concentration of the potential antagonist compound in the culture medium, thereby indicating the ability of the potential antagonist compound to inhibit activation of the polypeptide encoded by, the polynucleotide sequence described herein.

The present invention further relates to a bioassay for evaluating compounds as potential agonists for a polypeptide encoded by the polynucleotide sequence described herein, the bioassay may comprise:
a) culturing test cells in culture medium containing increasing concentrations of at least one compound whose ability to promote the action of the polypeptide encoded by the polynucleotide sequence described herein is sought to be determined, wherein the test cells may contain a polynucleotide sequence described herein (for example, in a form having improved trans-activation transcription activity, relative to wild-type polynucleotide, and comprising a response element operatively linked to a reporter gene); and thereafter
b) monitoring in the cells the level of expression of the product of the reporter gene as a function of the concentration of the potential agonist compound in the culture medium, thereby indicating the ability of the potential agonist compound to promote activation of a polypeptide encoded by the polynucleotide sequence described herein.

### Use of NSEQ as a Identification Tool or as a Diagnostic Screening Tool

The skilled artisan will readily recognize that NSEQ may be used to identify a particular cell, cell type, tissue, disease and thus may be used for diagnostic purposes to determine the absence, presence, or altered expression (i.e. increased or decreased compared to normal) of the expression product of a gene. Suitable NSEQ may be for example, between 10 and 20 or longer, i.e., at least 10 nucleotides long or at least 12 nucleotides long, or at least 15 nucleotides long up to any desired length and may comprise, for example, RNA, DNA, branched nucleic acids, and/or peptide nucleic acids (PNAs). In one alternative, the polynucleotides may be used to detect and quantify gene expression in samples in which expression of NSEQ is correlated with disease. In another alternative, NSEQ may be used to detect genetic polymorphisms associated with a disease. These polymorphisms may be detected, for example, in the transcript, cDNA or genomic DNA.

The invention provides for the use of at least one of the NSEQ described herein on an array and for the use of that array in a method of detection of a particular cell, cell type, tissue, disease for the prognosis or diagnosis of cancer. The method may comprise hybridizing the array with a patient sample (putatively comprising or comprising a target polynucleotide sequence substantially complementary to a NSEQ) under conditions to allow complex formation (between NSEQ and target polynucleotide), detecting complex formation, wherein the complex formation is indicative of the presence of the polynucleotide and wherein the absence of complex formation is indicative of the absence of the polynucleotide in the patient sample. The presence or absence of the polynucleotide may be indicative of cancer such as, for example, ovarian cancer or other cancer as indicated herein.

The method may also comprise the step of quantitatively or qualitatively comparing (e.g., with a computer system, apparatus) the level of complex formation in the patient sample to that of standards for normal cells or individual or other type, origin or grade of cancer.

The present invention provides one or more compartmentalized kits for detection of a polynucleotide and/or polypeptide for the diagnosis or prognosis of ovarian cancer. A first kit may have a receptacle containing at least one isolated NSEQ or probe comprising NSEQ. Such a probe may bind to a nucleic acid fragment that is present/absent in normal cells but which is absent/present in affected or diseased cells. Such a probe may be specific for a nucleic acid site that is normally active/inactive but which may be inactive/active in certain cell types. Similarly, such a probe may be specific for a nucleic acid site that may be abnormally expressed in certain cell types. Finally, such a probe may identify a specific mutation. The probe may be capable of hybridizing to the nucleic acid sequence that is mutated (not identical to the normal nucleic acid sequence), or may be capable of hybridizing to nucleic acid sequences adjacent to the mutated nucleic acid sequences. The probes provided in the present kits may have a covalently attached reporter molecule. Probes and reporter molecules may be readily prepared as described above by those of skill in the art.

Antibodies (e.g., isolated antibody) that may specifically bind to a protein or polypeptide described herein (a PSEQ) as well as nucleic acids encoding such antibodies are also encompassed by the present invention.

As used herein the term “antibody” means a monoclonal antibody, a polyclonal antibody, a single chain antibody, a chimeric antibody, a humanized antibody, a deimmunized antibody, an antigen-binding fragment, an Fab fragment; an F(ab′)₂ fragment, and Fv fragment; CDRs, or a single-chain antibody comprising an antigen-binding fragment (e.g., a single chain Fv).

The antibody may originate for example, from a mouse, rat or any other mammal or from other sources such as through recombinant DNA technologies.

The antibody may also be a human antibody which may be obtained, for example, from a transgenic non-human mammal capable of expressing human Ig genes. The antibody may also be a humanized antibody which may comprise, for example, one or more complementarity determining regions of non-human origin. It may also comprise a surface residue of a human antibody and/or framework regions of a human antibody. The antibody may also be a chimeric antibody which may comprise, for example, variable domains of a non-human antibody and constant domains of a human antibody.

The antibody of the present invention may be mutated and selected based on an increased affinity, solubility, stability, specificity and/or for one of a polypeptide described herein and/or based on a reduced immunogenicity in a desired host or for other desirable characteristics.

Suitable antibodies may bind to unique antigenic regions or epitopes in the polypeptides, or a portion thereof. Epitopes and antigenic regions useful for generating antibodies may be found within the proteins, polypeptides or peptides by procedures available to one of skill in the art. For example, short, unique peptide sequences may be identified in the proteins and polypeptides that have little or no homology to known amino acid sequences. Preferably the region of a protein selected to act as a peptide epitope or antigen is not entirely hydrophobic; hydrophilic regions are preferred because those regions likely constitute surface epitopes rather than internal regions of the proteins and polypeptides. These surface epitopes are more readily detected in samples tested for the presence of the proteins and polypeptides. Such antibodies may include, but are not limited to, polyclonal, monoclonal, chimeric, and single chain antibodies, Fab fragments, and fragments produced by a Fab expression library. The production of antibodies is well known to one of skill in the art and is not intended to be limited herein.

Peptides may be made by any procedure known to one of skill in the art, for example, by using in vitro translation or chemical synthesis procedures or by introducing a suitable expression vector into cells. Short peptides which provide an antigenic epitope but which by themselves are too small to induce an immune response may be conjugated to a suitable carrier. Suitable carriers and methods of linkage are well known in the art. Suitable carriers are typically large macromolecules such as proteins, polysaccharides and polymeric amino acids. Examples include serum albumins, keyhole limpet hemocyanin, ovalbumin, polylysine and the like. One of skill in the art may use available procedures and coupling reagents to link the desired peptide epitope to such a carrier. For example, coupling reagents may be used to form disulfide linkages or thioether linkages from the carrier to the peptide of interest. If the peptide lacks a disulfide group, one may be provided by the addition of a cysteine residue. Alternatively, coupling may be accomplished by activation of carboxyl groups.

The minimum size of peptides useful for obtaining antigen specific antibodies may vary widely. The minimum size must be sufficient to provide an antigenic epitope that is specific to the protein or polypeptide. The maximum size is not critical unless it is desired to obtain antibodies to one particular epitope. For example, a large polypeptide may comprise multiple epitopes, one epitope being particularly useful and a second epitope being immunodominant, etc. Typically, antigenic peptides selected from the present proteins and polypeptides will range without limitation, from 5 to about 100 amino acids in length. More typically, however, such an antigenic peptide will be a maximum of about 50 amino acids in length, and preferably a maximum of about 30 amino acids. It is usually desirable to select a sequence of about 6, 8, 10, 12 or 15 amino acids, up to about 20 or 25 amino acids (and any number therebetween).

Amino acid sequences comprising useful epitopes may be identified in a number of ways. For example, preparing a series of short peptides that taken together span the entire protein sequence may be used to screen the entire protein sequence. One of skill in the art may routinely test a few large polypeptides for the presence of an epitope showing a desired reactivity and also test progressively smaller and overlapping fragments to identify a preferred epitope with the desired specificity and reactivity.

As mentioned herein, antigenic polypeptides and peptides are useful for the production of monoclonal and polyclonal antibodies. Antibodies to a polypeptide encoded by the polynucleotides of NSEQ, polypeptide analogs or portions thereof, may be generated using methods that are well known in the art. For example, monoclonal antibodies may be prepared using any technique that provides for the production of antibody molecules by continuous cell lines in culture. These include, but are not limited to, the hybridoma, the human B-cell hybridoma, and the EBV-hybridoma techniques. In addition, techniques developed for the production of chimeric antibodies may be used. Alternatively, techniques described for the production of single chain antibodies may be employed. Fabs that may contain specific binding sites for a polypeptide encoded by the polynucleotides of NSEQ, or a portion thereof, may also be generated. Various immunoassays may be used to identify antibodies having the desired specificity. Numerous protocols for competitive binding or immunoradiometric assays using either polyclonal or monoclonal antibodies with established specificities are well known in the art.

To obtain polyclonal antibodies, a selected animal may be immunized with a protein or polypeptide. Serum from the animal may be collected and treated according to known procedures. Polyclonal antibodies to the protein or polypeptide of interest may then be purified by affinity chromatography. Techniques for producing polyclonal antisera are well known in the art.

Monoclonal antibodies (MAbs) may be made by one of several procedures available to one of skill in the art, for example, by fusing antibody producing cells with immortalized cells and thereby making a hybridoma. The general methodology for fusion of antibody producing B cells to an immortal cell line is well within the province of one skilled in the art. Another example is the generation of MAbs from mRNA extracted from bone marrow and spleen cells of immunized animals using combinatorial antibody library technology.

One drawback of MAbs derived from animals or from derived cell lines is that although they may be administered to a patient for diagnostic or therapeutic purposes, they are often recognized as foreign antigens by the immune system and are unsuitable for continued use. Antibodies that are not recognized as foreign antigens by the human immune system have greater potential for both diagnosis and treatment. Methods for generating human and humanized antibodies are now well known in the art.

Chimeric antibodies may be constructed in which regions of a non-human MAb are replaced by their human counterparts. A preferred chimeric antibody is one that has amino acid sequences that comprise one or more complementarity determining regions (CDRs) of a non-human Mab that binds to a polypeptide encoded by the polynucleotides of NSEQ, or a portion thereof, grafted to human framework (FW) regions. Methods for producing such antibodies are well known in the art. Amino acid residues corresponding to CDRs and FWs are known to one of average skill in the art.

A variety of methods have been developed to preserve or to enhance affinity for antigen of antibodies comprising grafted CDRs. One way is to include in the chimeric antibody the foreign framework residues that influence the conformation of the CDR regions. A second way is to graft the foreign CDRs onto human variable domains with the closest homology to the foreign variable region. Thus, grafting of one or more non-human CDRs onto a human antibody may also involve the substitution of amino acid residues which are adjacent to a particular CDR sequence or which are not contiguous with the CDR sequence but which are packed against the CDR in the overall antibody variable domain structure and which affect the conformation of the CDR. Humanized antibodies of the invention therefore include human antibodies which comprise one or more non-human CDRs as well as such antibodies in which additional substitutions or replacements have been made to preserve or enhance binding characteristics.

Chimeric antibodies of the invention also include antibodies that have been humanized by replacing surface-exposed residues to make the MAb appear human. Because the internal packing of amino acid residues in the vicinity of the antigen-binding site remains unchanged, affinity is preserved. Substitution of surface-exposed residues of a polypeptide encoded by the polynucleotides of NSEQ (or a portion thereof)-antibody according to the invention for the purpose of humanization does not mean substitution of CDR residues or adjacent residues that influence affinity for a polypeptide encoded by the polynucleotides of NSEQ, or a portion thereof.

Chimeric antibodies may also include antibodies where some or all non-human constant domains have been replaced with human counterparts. This approach has the advantage that the antigen-binding site remains unaffected. However, significant amounts of non-human sequences may be present where variable domains are derived entirely from non-human antibodies.

Antibodies of the invention include human antibodies that are antibodies consisting essentially of human sequences. Human antibodies may be obtained from phage display libraries wherein combinations of human heavy and light chain variable domains are displayed on the surface of filamentous phage. Combinations of variable domains are typically displayed on filamentous phage in the form of Fab′ s or scFvs. The library may be screened for phage bearing combinations of variable domains having desired antigen-binding characteristics. Preferred variable domain combinations are characterized by high affinity for a polypeptide encoded by the polynucleotides of NSEQ, or a portion thereof. Preferred variable domain combinations may also be characterized by high specificity for a polypeptide encoded by the polynucleotides of NSEQ, or a portion thereof, and little cross-reactivity to other related antigens. By screening from very large repertoires of antibody fragments, (2−10×10¹⁰) a good diversity of high affinity Mabs may be isolated, with many expected to have sub-nanomolar affinities for a polypeptide encoded by the polynucleotides of NSEQ, or a portion thereof.

Alternatively, human antibodies may be obtained from transgenic animals into which un-rearranged human Ig gene segments have been introduced and in which the endogenous mouse Ig genes have been inactivated. Preferred transgenic animals contain very large contiguous Ig gene fragments that are over 1 Mb in size but human polypeptide-specific Mabs of moderate affinity may be raised from transgenic animals containing smaller gene loci. Transgenic animals capable of expressing only human Ig genes may also be used to raise polyclonal antiserum comprising antibodies solely of human origin.

Antibodies of the invention may include those for which binding characteristics have been improved by direct mutation or by methods of affinity maturation. Affinity and specificity may be modified or improved by mutating CDRs and screening for antigen binding sites having the desired characteristics. CDRs may be mutated in a variety of ways. One way is to randomize individual residues or combinations of residues so that in a population of otherwise identical antigen binding sites, all twenty amino acids may be found at particular positions. Alternatively, mutations may be induced over a range of CDR residues by error prone PCR methods. Phage display vectors containing heavy and light chain variable region gene may be propagated in mutator strains of E. coli. These methods of mutagenesis are illustrative of the many methods known to one of skill in the art.

The antibody may further comprise a detectable label (reporter molecule) attached thereto.

There is provided also methods of producing antibodies able to specifically bind to one of a polypeptide, polypeptide fragments, or polypeptide analogs described herein, the method may comprise:
a) immunizing a mammal (e.g., mouse, a transgenic mammal capable of producing human Ig, etc.) with a suitable amount of a PSEQ described herein including, for example, a polypeptide fragment comprising at least 6 (e.g., 8, 10, 12 etc.) consecutive amino acids of a PSEQ;
b) collecting the serum from the mammal; and
c) isolating the polypeptide-specific antibodies from the serum of the mammal.

The method may further comprise the step of administering a second dose to the mammal (e.g., animal).

Methods of producing a hybridoma which secretes an antibody that specifically binds to a polypeptide are also encompassed herewith and are known in the art.

The method may comprise:
a) immunizing a mammal (e.g., mouse, a transgenic mammal capable of producing human Ig, etc.) with a suitable amount of a PSEQ thereof;
b) obtaining lymphoid cells from the immunized animal obtained from (a);
c) fusing the lymphoid cells with an immortalizing cell to produce hybrid cells; and
d) selecting hybrid cells which produce antibody that specifically binds to a PSEQ thereof.

Also encompassed by the present invention is a method of producing an antibody that specifically binds to one of the polypeptide described herein, the method may comprise:
a) synthesizing a library of antibodies (e.g., antigen binding fragment) on phage or ribosomes;
b) panning the library against a sample by bringing the phage or ribosomes into contact with a composition comprising a polypeptide or polypeptide fragment described herein;
c) isolating phage which binds to the polypeptide or polypeptide fragment, and;
d) obtaining an antibody from the phage or ribosomes.

The antibody of the present invention may thus be obtained, for example, from a library (e.g., bacteriophage library) which may be prepared, for example, by
a) extracting cells which are responsible for production of antibodies from a host mammal;
b) isolating RNA from the cells of (a);
c) reverse transcribing mRNA to produce cDNA;
d) amplifying the cDNA using a (antibody-specific) primer; and
e) inserting the cDNA of (d) into a phage display vector or ribosome display cassette such that antibodies are expressed on the phage or ribosomes.

In order to generate antibodies, the host animal may be immunized with polypeptide and/or a polypeptide fragment and/or analog described herein to induce an immune response prior to extracting the cells that are responsible for production of antibodies.

The antibodies obtained by the means described herein may be useful for detecting proteins, variant and derivative polypeptides in specific tissues or in body fluids. Moreover, detection of aberrantly expressed proteins or protein fragments is probative of a disease state. For example, expression of the present polypeptides encoded by the polynucleotides of NSEQ, or a portion thereof, may indicate that the protein is being expressed at an inappropriate rate or at an inappropriate developmental stage. Hence, the present antibodies may be useful for detecting diseases associated with protein expression from NSEQs disclosed herein.

For in vivo detection purposes, antibodies may be those that preferably recognize an epitope present at the surface of a tumor cell.

A variety of protocols for measuring polypeptides, including ELISAs, RIAs, and FACS, are well known in the art and provide a basis for diagnosing altered or abnormal levels of expression. Standard values for polypeptide expression are established by combining samples taken from healthy subjects, preferably human, with antibody to the polypeptide under conditions for complex formation. The amount of complex formation may be quantified by various methods, such as photometric means. Quantities of polypeptide expressed in disease samples may be compared with standard values. Deviation between standard and subject values may establish the parameters for diagnosing or monitoring disease.

Design of immunoassays is subject to a great deal of variation and a variety of these are known in the art. Immunoassays may use a monoclonal or polyclonal antibody reagent that is directed against one epitope of the antigen being assayed. Alternatively, a combination of monoclonal or polyclonal antibodies may be used which are directed against more than one epitope. Protocols may be based, for example, upon competition where one may use competitive drug screening assays in which neutralizing antibodies capable of binding a polypeptide encoded by the polynucleotides of NSEQ, or a portion thereof, specifically compete with a test compound for binding the polypeptide. Alternatively one may use, direct antigen-antibody reactions or sandwich type assays and protocols may, for example, make use of solid supports or immunoprecipitation. Furthermore, antibodies may be labelled with a reporter molecule for easy detection. Assays that amplify the signal from a bound reagent are also known. Examples include immunoassays that utilize avidin and biotin, or which utilize enzyme-labelled antibody or antigen conjugates, such as ELISA assays.

Kits suitable for immunodiagnosis and containing the appropriate labelled reagents include antibodies directed against the polypeptide protein epitopes or antigenic regions, packaged appropriately with the remaining reagents and materials required for the conduct of the assay, as well as a suitable set of assay instructions.

The present invention therefore provides a kit for specifically detecting a polypeptide described herein, the kit may comprise, for example, an antibody or antibody fragment capable of binding specifically to the polypeptide described herein.

In accordance with the present invention, the kit may be a diagnostic kit, which may comprise:
a) one or more antibodies described herein; and
b) a detection reagent which may comprise a reporter group.

In accordance with the present invention, the antibodies may be immobilized on a solid support. The detection reagent may comprise, for example, an anti-immunoglobulin, protein G, protein A or lectin etc. The reporter group may be selected, without limitation, from the group consisting of radioisotopes, fluorescent groups, luminescent groups, enzymes, biotin and dye particles

### Use of NSEQ, PSEQ as a Therapeutic or Therapeutic Targets

One of skill in the art will readily appreciate that the NSEQ, PSEQ, expression systems, assays, kits and array discussed above may also be used to evaluate the efficacy of a particular therapeutic treatment regimen, in animal studies, in clinical trials, or to monitor the treatment of an individual subject. Once the presence of disease is established and a treatment protocol is initiated, hybridization or amplification assays may be repeated on a regular basis to determine if the level of mRNA or protein in the patient (patient's blood, tissue, cell etc.) begins to approximate the level observed in a healthy subject. The results obtained from successive assays may be used to show the efficacy of treatment over a period ranging from several days to many years.

In yet another aspect of the invention, NSEQ may be used therapeutically for the purpose of expressing mRNA and polypeptide, or conversely to block transcription and/or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art may be used to construct vectors to express nucleic acid sequences or their complements.

Alternatively, NSEQ may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of NSEQ by transfection, liposome injections, or polycationic amino polymers may be achieved using methods that are well known in the art. Additionally, endogenous NSEQ expression may be inactivated using homologous recombination methods that insert an inactive gene sequence into the coding region or other targeted region of NSEQ.

Depending on the specific goal to be achieved, vectors containing NSEQ may be introduced into a cell or tissue to express a missing polypeptide or to replace a non-functional polypeptide. Of course, when one wishes to express PSEQ in a cell or tissue, one may use a NSEQ able to encode such PSEQ for that purpose or may directly administer PSEQ to that cell or tissue.

On the other hand, when one wishes to attenuate or inhibit the expression of PSEQ, one may use a NSEQ (e.g., an inhibitory NSEQ) that is substantially complementary to at least a portion of a NSEQ able to encode such PSEQ.

The expression of an inhibitory NSEQ may be done by cloning the inhibitory NSEQ into a vector and introducing the vector into a cell to down-regulate the expression of a polypeptide encoded by the target NSEQ. Complementary or anti-sense sequences may also comprise an oligonucleotide derived from the transcription initiation site; nucleotides between about positions −10 and +10 from the ATG may be used. Therefore, inhibitory NSEQ may encompass a portion that is substantially complementary to a desired nucleic acid molecule to be inhibited and a portion (sequence) which binds to an untranslated portion of the nucleic acid.

Similarly, inhibition may be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee et al. 1994)

Ribozymes, enzymatic RNA molecules, may also be used to catalyze the cleavage of mRNA and decrease the levels of particular mRNAs, such as those comprising the polynucleotide sequences of the invention. Ribozymes may cleave mRNA at specific cleavage sites. Alternatively, ribozymes may cleave mRNAs at locations dictated by flanking regions that form complementary base pairs with the target mRNA. The construction and production of ribozymes is well known in the art.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, may be included.

Pharmaceutical compositions are also encompassed by the present invention. The pharmaceutical composition may comprise at least one NSEQ or PSEQ and a pharmaceutically acceptable carrier.

As it will be appreciated form those of skill in the art, the specificity of expression NSEQ and/or PSEQ in tumor cells may advantageously be used for inducing an immune response (through their administration) in an individual having, or suspected of having a tumor expressing such sequence. Administration of NSEQ and/or PSEQ in individuals at risk of developing a tumor expressing such sequence is also encompassed herewith.

In addition to the active ingredients, a pharmaceutical composition may contain pharmaceutically acceptable carriers comprising excipients and auxiliaries that facilitate processing of the active compounds into preparations that may be used pharmaceutically.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and most preferably, humans.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

The term “treatment” for purposes of this disclosure refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

### Use of NSEQ in General Research

The invention also provides products, compositions, processes and methods that utilize a NSEQ described herein, a polypeptide encoded by a NSEQ described herein, a PSEQ described herein for research, biological, clinical and therapeutic purposes. For example, to identify splice variants, mutations, and polymorphisms and to generate diagnostic and prognostic tools.

NSEQ may be extended utilizing a partial nucleotide sequence and employing various PCR-based methods known in the art to detect upstream sequences such as promoters and other regulatory elements. Additionally, one may use an XL-PCR kit (PE Biosystems, Foster City Calif.), nested primers, and commercially available cDNA libraries (Life Technologies, Rockville Md.) or genomic libraries (Clontech, Palo Alto Calif.) to extend the sequence.

The polynucleotides (NSEQ) may also be used as targets in a microarray. The microarray may be used to monitor the expression patterns of large numbers of genes simultaneously and to identify splice variants, mutations, and polymorphisms. Information derived from analyses of the expression patterns may be used to determine gene function, to identify a particular cell, cell type or tissue, to understand the genetic basis of a disease, to diagnose a disease, and to develop and monitor the activities of therapeutic agents used to treat a disease. Microarrays may also be used to detect genetic diversity, single nucleotide polymorphisms which may characterize a particular population, at the genomic level.

The polynucleotides (NSEQ) may also be used to generate hybridization probes useful in mapping the naturally occurring genomic sequence. Fluorescent in situ hybridization (FISH) may be correlated with other physical chromosome mapping techniques and genetic map data.

It is to be understood herein that a sequence which is upregulated in an ovarian cancer cell (e.g., malignant ovarian cancer cell) may represent a sequence which is involved in or responsible for the growth, development, malignancy and so on, of the cancer cell (referred herein as a positive regulator of ovarian cancer). It is also to be understood that a sequence which is downregulated (unexpressed or expressed at low levels) in a malignant ovarian cancer cell may represent a sequence which is responsible for the maintenance of the normal status (untransformed) of an ovarian cell (referred herein as a negative regulator of ovarian cancer). Therefore, both the presence or absence of some sequences may be indicative of the disease or may be indicative of the disease, probability of having a disease, degree of severity of the disease (staging).

Therefore, the present invention relates in an aspect thereof to an isolated polynucleotide (e.g., exogenous form of) that may comprise a member selected from the group consisting of;
a) a polynucleotide which may comprise or consist of SEQ ID NO.:1,
b) a polynucleotide which may comprise the open reading frame of SEQ ID NO.:1,
c) a polynucleotide which may comprise a transcribed or transcribable portion of SEQ ID NO.:1, which may be, for example, free of untranslated or untranslatable portion(s),
d) a polynucleotide which may comprise a translated or translatable portion of any one of SEQ ID NO.:1 (e.g., coding portion),
e) a polynucleotide which may comprise a sequence substantially identical (e.g., from about 50 to 100%, or about 60 to 100% or about 70 to 100% or about 80 to 100% or about 85, 90, 95 to 100% identical over the entire sequence or portion of sequences) to a), b), c), or d);
f) a polynucleotide which may comprise a sequence substantially complementary (e.g., from about 50 to 100%, or about 60 to 100% or about 70 to 100% or about 80 to 100% or about 85, 90, 95 to 100% complementarity over the entire sequence or portion of sequences) to a), b), c), or d) and;
g) a fragment of any one of a) to f)
   including polynucleotides which consist in the above.

More specifically, the present invention relates to expressed polynucleotides which are selected from the group consisting of;
a) a polynucleotide which may comprise or consist of SEQ ID NO.:1,
b) a polynucleotide which may comprise the open reading frame of SEQ ID NO.:1,
c) a polynucleotide which may comprise a transcribed or transcribable portion of SEQ ID NO.:1, which may be, for example, free of untranslated or untranslatable portion(s),
d) a polynucleotide which may comprise a translated or translatable portion of SEQ ID NO.:1, (e.g., coding portion),
e) a polynucleotide which may comprise a sequence substantially identical (e.g., from about 50 to 100%, or about 60 to 100% or about 70 to 100% or about 80 to 100% or about 85, 90, 95 to 100% identical over the entire sequence or portion of sequences) to a), b), c), or d);
f) a polynucleotide which may comprise a sequence substantially complementary (e.g., from about 50 to 100%, or about 60 to 100% or about 70 to 100% or about 80 to 100% or about 85, 90, 95 to 100% complementarity over the entire sequence or portion of sequences) to a), b), c), or d) and;
g) a fragment of any one of a) to f)
   including polynucleotides which consist in the above.

Vectors (e.g., a viral vector, a mammalian vector, a plasmid, a cosmid, etc.) that may comprise the polynucleotides described herein are also encompassed by the present invention. The vector may be, for example, an expression vector.

The present invention also provides a library of polynucleotide comprising at least one polynucleotide (e.g., at least two, etc.) described herein. The library may be, for example, an expression library. Some or all of the polynucleotides described herein may be contained within an expression vector. The present invention also relates to a polypeptide library that may comprise at least one (e.g., at least two, etc.) polypeptide as described herein.

In another aspect, the present invention provides arrays that may comprise at least one polynucleotide (e.g., at least two, etc.) described herein.

The present invention also provides an isolated cell (e.g., an isolated live cell such as an isolated mammalian cell, a bacterial cell, a yeast cell, an insect cell, etc.) that may comprise the polynucleotide, the vector or the polypeptide described herein.

In yet a further aspect the present invention relates to a composition comprising the polynucleotide and/or polypeptide described herein.

In accordance with the present invention, the composition may be, for example, a pharmaceutical composition that may comprise a polynucleotide and/or a polypeptide described herein and a pharmaceutically acceptable carrier. More specifically, the pharmaceutical composition may be used for the treatment of ovarian cancer and/or for inhibiting the growth of an ovarian cancer cell.

Polynucleotides fragments of those listed above includes polynucleotides comprising at least 10 nucleic acids which may be identical to a corresponding portion of any one of a) to e) and more particularly a coding portion of SEQ ID NO.:1.

Another exemplary embodiment of polynucleotide fragments encompassed by the present invention includes polynucleotides comprising at least 10 nucleic acids which may be substantially complementary to a corresponding portion of a coding portion of SEQ ID NO.:1 and encompasses, for example, fragments such as those defined by SEQ ID NO.:44 or 45.

These above sequences may represent powerful markers of cancer and more particularly of, ovarian cancer, breast cancer, prostate cancer, leukemia, melanoma, renal cancer, colon cancer, lung cancer, cancer of the central nervous system and any combination thereof.

Based on the results presented herein and upon reading the present description, a person skilled in the art will understand that the appearance of a positive signal upon testing (hybridization, PCR amplification etc.) for the presence of a given sequence amongst those expressed in a cancer cell, indicates that such sequence is specifically expressed in that type of cancer cell. A person skilled in the art will also understand that, sequences that are specifically expressed in a certain types of cancer cell may be used for developing tools for the detection of this specific type of cancer cell and may also be used as targets in the development of anticancer drugs.

A positive signal may be in the form of a band in a gel following electrophoresis, Northern blot or Western blot, a PCR fragment detected by emission of fluorescence, etc.

As it will be understood, sequences that are particularly useful for the development of tools for the detection of cancer cell may preferably be expressed at lower levels in at least some normal cells (non-cancerous cells).

For example, in Figures and related description, the appearance of a band upon RT-PCR amplification of mRNAs obtained from ovarian cancer cells, renal cancer cells, lung cancer cells, breast cancer cells and melanoma cells indicates that the relevant sequence is expressed in such cancer cells and that this sequence may therefore represent a valid marker and target for these types of cancer cells.

NSEQs chosen among those that are substantially complementary to those described herein, or to fragments thereof may be used for the treatment of cancer.

The present invention therefore relates to a method for identifying a cancer cell. The method may comprise contacting a cell, a cell sample (cell lysate), a body fluid (blood, urine, plasma, saliva etc.) or a tissue with a reagent which may be, for example, capable of specifically binding at least one NSEQ or PSEQ described herein. The method may more particularly comprise contacting a sequence isolated or derived such cell, sample, fluid or tissue. The complex formed may be detected using methods known in the art.

In accordance with the present invention, the presence of the above mentioned complex may be indicative (a positive indication of the presence) of the presence of a cancer cell.

The present invention also relates in an additional aspect thereof to a method for the diagnosis or prognosis of cancer. The method may comprise, for example, detecting, in a cell, tissue, sample, body fluid, etc., at least one NSEQ or PSEQ described herein.

The cell, cell sample, body fluid or tissue may originate, for example, from an individual which has or is suspected of having a cancer and more particularly ovarian cancer, breast cancer, prostate cancer, leukemia, melanoma, renal cancer, colon cancer, lung cancer and/or cancer of the central nervous system

Any of the above mentioned methods may further comprise comparing the level obtained with at least one reference level or value.

Detection of NSEQ may require an amplification (e.g., PCR) step in order to have sufficient material for detection purposes.

In accordance with the present invention, the polynucleotide described herein may comprise, for example, a RNA molecule, a DNA molecule, including those that are partial or complete, single-stranded or double-stranded, hybrids, modified by a group etc.

Other aspects of the present invention which are encompassed herewith comprises the use of at least one NSEQ or PSEQ described herein and derived antibodies in the manufacture of a composition for identification or detection of a cancer cell (e.g., a tumor cell) or for inhibiting or lowering the growth of cancer cell (e.g., for treatment of ovarian cancer or other cancer).

As some NSEQ and PSEQ are expressed at higher levels in malignant ovarian cancer than in LMP detection of such NSEQ or PSEQ in a sample from an individual (or in vivo) one may rule-out a low malignant potential ovarian cancer and may therefore conclude in a diagnostic of a malignant ovarian cancer. Furthermore, detection of the NSEQ or PSEQ in a cell, tissue, sample or body fluid from an individual may also be indicative of a late-stage malignant ovarian cancer. As such, therapies adapted for the treatment of a malignant ovarian cancer or a late-stage malignant ovarian cancer may be commenced.

In accordance with an embodiment of the present invention, the method may also comprise a step of qualitatively or quantitatively comparing the level (amount, presence) of at least one complex present in the test cell, test sample, test fluid or test tissue with the level of complex in a normal cell, a normal cell sample, a normal body fluid, a normal tissue or a reference value (e.g., for a non-cancerous condition).

The normal cell may be any cell that does not substantially express the desired sequence to be detected. Examples of such normal cells are included for example, in the description of the drawings section. A normal cell sample or tissue thus include, for example, a normal (non-cancerous) ovarian cell, a normal breast cell, a normal prostate cell, a normal lymphocyte, a normal skin cell, a normal renal cell, a normal colon cell, a normal lung cell and/or a normal cell of the central nervous system. For comparison purposes, a normal cell may be chosen from those of identical or similar cell type.

Of course, the presence of more than one complex may be performed in order to increase the precision of the diagnostic method. As such, at least two complexes (e.g., formed by a first reagent and a first polynucleotide and a second reagent or a second polynucleotide) or multiple complexes may be detected.

An exemplary embodiment of a reagent which may be used for detecting a NSEQ described herein is a polynucleotide which may comprise a sequence substantially complementary to the NSEQ.

A suitable reference level or value may be, for example, derived from the level of expression of a specified sequence in a low malignant potential ovarian cancer and/or from a normal cell.

It will be understood herein that a higher level of expression measured in a cancer cell, tissue or sample in comparison with a reference value or sample is a indicative of the presence of cancer in the tested individual.

For example, the higher level measured in an ovarian cell, ovarian tissue or a sample of ovarian origin compared to a reference level or value for a normal cell (normal ovarian cell or normal non-ovarian cell) may be indicative of an ovarian cancer.

For comparison purpose, the presence or level of expression of a desired NSEQ or PSEQ to be detected or identified may be compared with the presence, level of expression, found in a normal cell that has been shown herein not to express the desired sequence.

Therapeutic uses and methods are also encompassed herewith.

The invention therefore provides polynucleotides that may be able to lower or inhibit the growth of an ovarian cancer cell (e.g., in a mammal or mammalian cell thereof).

The present invention therefore relates in a further aspect to the use of a polynucleotide sequence that may be selected from the group consisting of
a) a polynucleotide which may comprise a sequence substantially complementary to SEQ ID NO.:1,
b) a polynucleotide which may comprise a sequence substantially complementary to a transcribed or transcribable portion of SEQ ID NO.:1,
c) a polynucleotide which may comprise a sequence substantially complementary to a translated or translatable portion of SEQ ID NO.:1, and;
d) a fragment of any one of a) to c) for reducing, lowering or inhibiting the growth of a cancer cell.

The polynucleotide may be selected, for example, from the group consisting of polynucleotides which may comprise a sequence of at least 10 nucleotides which is complementary to the nucleic acid sequence of SEQ ID NO.:1 (to a translated portion which may be free, for example, of untranslated portions).

Of course, the present invention encompasses immunizing an individual by administering a NSEQ (e.g., in an expression vector) or a PSEQ.

The present invention also relates to a method of reducing or slowing the growth of an ovarian cancer cell in an individual in need thereof. The method may comprise administering to the individual a polynucleotide sequence that may be selected from the group consisting of:
a) a polynucleotide which may comprise a sequence substantially complementary (also including 100% complementary over a portion, e.g., a perfect match) to SEQ ID NO.:1,
b) a polynucleotide which may comprise a sequence substantially complementary (also including 100% complementary over a portion, e.g., a perfect match) to a transcribed or transcribable portion of SEQ ID NO.:1,
c) a polynucleotide which may comprise a sequence substantially complementary (also including 100% complementary over a portion, e.g., a perfect match) to a translated or translatable portion of SEQ ID NO.:1, and;
d) a fragment of any one of a) to c).

The present invention therefore provides in yet another aspect thereof, a siRNA or shRNA molecule that is able to lower the expression of a nucleic acid selected from the group consisting of:
a) a polynucleotide which may comprise SEQ ID NO.:1,
b) a polynucleotide which may comprise a transcribed or transcribable portion of SEQ ID NO.:1,
c) a polynucleotide which may comprise a translated or translatable portion of SEQ ID NO.:1, and;
d) a polynucleotide which may comprise a sequence substantially identical to a), b), or c).

Exemplary embodiment of polynucleotides are those which, for example, may be able to inhibit the growth of an ovarian cancer cell, such as, for example, a polynucleotide having or comprising a sequence such as those defined by SEQ ID NO.:44 or 45. These specific sequences are provided as guidance only and are not intended to limit the scope of the invention.

The present invention also provides a kit for the diagnosis of cancer. The kit may comprise at least one polynucleotide as described herein and/or a reagent capable of specifically binding at least one polynucleotide described herein.

In a further aspect, the present invention relates to an isolated polypeptide encoded by the polynucleotide described herein.

The present invention more particularly provides an isolated polypeptide that may be selected from the group consisting of:
a) a polypeptide which may comprise SEQ ID NO.:2
b) a polypeptide which may be encoded by any one of the polynucleotide described herein,
c) a fragment of any one of a) or b),
d) a derivative of any one of a) or b) and;
e) an analog of any one of a) or b).

In accordance with the present invention, the analog may comprise, for example, at least one amino acid substitution, deletion or insertion in its amino acid sequence.

The substitution may be conservative or non-conservative. The polypeptide analog may be a biologically active analog or an immunogenic analog which may comprise, for example, at least one amino acid substitution (conservative or non conservative), for example, 1 to 5, 1 to 10, 1 to 15, 1 to 20, 1 to 50 etc. (including any number there between) compared to the original sequence. An immunogenic analog may comprise, for example, at least one amino acid substitution compared to the original sequence and may still be bound by an antibody specific for the original sequence. In accordance with the present invention, a polypeptide fragment may comprise, for example, at least 6 consecutive amino acids, at least 8 consecutive amino acids or more of an amino acid sequence selected from the group consisting of polypeptides encoded by a polynucleotide of SEQ ID NO.:1, including variants and analogs thereof. The fragment may be immunogenic and may be used for the purpose, for example, of generating antibodies.

Exemplary embodiments of polypeptide encompassed by the present invention are those which may be encoded by SEQ ID NO.:1.

In a further aspect the present invention relates to a polypeptide that may be encoded by the isolated differentially expressed sequence of the present invention. The present invention as well relates to the polypeptide encoded by the non-human ortholog polynucleotide, analogs, derivatives and fragments thereof.

A person skilled in the art may easily determine the possible peptide sequence encoded by a particular nucleic acid sequence as generally, a maximum of 6 possible open-reading frames exist in a particular coding sequence. The first possible open-reading frame may start at the first nucleotide (5′-3′) of the sequence, therefore using in a 5′ to 3′ direction nucleotides No. 1 to 3 as the first codon, using nucleotides 4 to 6 as the second codon, etc. The second possible open-reading frame may start at the second nucleotide (5′-3′) of the sequence, therefore using in a 5′ to 3′ direction nucleotides No. 2 to 4 as the first codon, using nucleotides 5 to 7 as the second codon, etc. Finally, the third possible open-reading frame may start at the third nucleotide (5′-3′) of the sequence, therefore using in a 5′ to 3′ direction nucleotides No. 3 to 5 as the first codon, using nucleotides 6 to 8 as the second codon, etc. The fourth possible open-reading frame may start at the first nucleotide of the sequence in a 3′ to 5′ direction, therefore using in 3′ to 5′ direction, nucleotides No. 1 to 3 as the first codon, using nucleotides 4 to 6 as the second codon, etc. The fifth possible open-reading frame may start at the second nucleotide of the sequence in a 3′ to 5′ direction, therefore using in a 3′ to 5′ direction, nucleotides No. 2 to 4 as the first codon, using nucleotides 5 to 7 as the second codon, etc. Finally, the sixth possible open-reading frame may start at the third nucleotide of the sequence in a 3′ to 5′ direction, therefore using in a 3′ to 5′ direction nucleotides No. 3 to 5 as the first codon, using nucleotides 6 to 8 as the second codon, etc.

In an additional aspect, the present invention relates to the use of at least one polypeptide in the manufacture of a composition for the identification or detection of a cancer cell (tumor cell). The polypeptide may be used, for example, as a standard in an assay and/or for detecting antibodies specific for the particular polypeptide, etc. In yet an additional aspect, the present invention relates to the use of at least one polypeptide described herein in the identification or detection of a cancer cell, such as for example, an ovarian cancer cell or any other cancer cell as described herein.

The present invention therefore relates in a further aspect, to the use of at least one polypeptide described herein in the prognosis or diagnosis of cancer, such as, for example, a malignant ovarian cancer or a low malignant potential ovarian cancer.

As such and in accordance with the present invention, detection of the polypeptide in a cell (e.g., ovarian cell), tissue (e.g., ovarian tissue), sample or body fluid from an individual may preferentially be indicative of a malignant ovarian cancer diagnosis over a low malignant potential ovarian cancer diagnosis and therefore may preferentially be indicative of a malignant ovarian cancer rather than a low malignant potential ovarian cancer.

Further in accordance with the present invention, the presence of the polypeptide in a cell, tissue, sample or body fluid from an individual may preferentially be indicative of a late-stage malignant ovarian cancer.

There is also provided by the present invention, methods for identifying a cancer cell, which may comprise, for example, contacting a test cell, a test cell sample (cell lysate), a test body fluid (blood, urine, plasma, saliva etc.) or a test tissue with a reagent which may be capable of specifically binding the polypeptide described herein, and detecting the complex formed by the polypeptide and reagent. The presence of a complex may be indicative (a positive indication of the presence) of a cancer cell such as for example, an ovarian cancer cell, a breast cancer cell, a prostate cancer cell, leukemia, melanoma, a renal cancer cell, a colon cancer cell, a lung cancer cell, a cancer cell of the central nervous system and any combination thereof.

The presence of a complex formed by the polypeptide and the specific reagent may be indicative, for example, of ovarian cancer including, for example, a low malignant potential ovarian cancer or a malignant ovarian cancer.

However, the method is more particularly powerful for the detection of ovarian cancer of the malignant type. Therefore, the presence of a complex may preferentially be indicative of a malignant ovarian cancer relative (rather than) to a low malignant potential ovarian cancer.

Detection of the complex may also be indicative of a late stage malignant ovarian cancer.

In accordance with the present invention, the method may also comprise a step of qualitatively or quantitatively comparing the level (amount, presence) of at least one complex present in a test cell, a test sample, a test fluid or a test tissue with the level of complex in a normal cell, a normal cell sample, a normal body fluid, a normal tissue or a reference value (e.g., for a non-cancerous condition).

Of course, the presence of more than one polypeptide or complex (two complexes or more (multiple complexes)) may be determined, e.g., one formed by a first specific reagent and a first polypeptide and another formed by a second specific reagent and a second polypeptide may be detected. Detection of more than one polypeptide or complex may help in the determination of the tumorigenicity of the cell.

An exemplary embodiment of a reagent, which may be used for the detection of the polypeptide described herein, is an antibody and antibody fragment thereof.

The present invention also relates to a kit that may comprise at least one of the polypeptide described herein and/or a reagent capable of specifically binding to at least one of the polypeptide described herein.

As one skill in the art will understand, compositions which comprises a polypeptide may be used, for example, for generating antibodies against the particular polypeptide, may be used as a reference for assays and kits, etc.

Additional aspects of the invention relate to isolated or purified antibodies (including an antigen-binding fragment thereof) which may be capable of specifically binding to a polypeptide selected from the group consisting of;
a) a polypeptide comprising or consisting of SEQ ID NO.:2, and;
b) a polypeptide comprising a polypeptide sequence encoded by any one of the polynucleotide sequence described herein (e.g., a fragment of at least 6 amino acids of the polypeptide).

More particularly, exemplary embodiments of the present invention relates to antibodies which may be capable of specifically binding a polypeptide comprising a polypeptide sequence encoded by SEQ ID NO.:2, or a fragment of at least 6 amino acids of the polypeptide.

In yet an additional aspect, the present invention relates to a hybridoma cell which is capable of producing an antibody which may specifically bind to a polypeptide selected from the group consisting of;
a) a polypeptide which may comprise SEQ ID NO.:2, and;
b) a polypeptide which may comprise a polypeptide sequence encoded by any one of the polynucleotide sequence described herein or a fragment of at least 6 amino acids of the polypeptide.

Exemplary hybridoma which are more particularly encompassed by the present invention are those which may produce an antibody which may be capable of specifically binding a polypeptide comprising a polypeptide sequence encoded by SEQ ID NO.:1 or a fragment of at least 6 amino acids of the polypeptide.

The present invention also relates to a composition that may comprise an antibody described herein.

In a further aspect the present invention provides a method of making an antibody which may comprise immunizing a non-human animal with an immunogenic fragment (at least 6 amino acids, at least 8 amino acids, etc.) of a polypeptide which may be selected, for example, from the group consisting of;
a) a polypeptide which may comprise or consist in SEQ ID NO.:2 or a fragment thereof, and;
b) a polypeptide which may comprise a polypeptide sequence encoded by any one of the polynucleotide sequence described herein or a portion thereof.

Exemplary polypeptides which may, more particularly, be used for generating antibodies are those which are encoded by SEQ ID NO.:1 (and polypeptide comprising a polypeptide fragment of these particular PSEQ). In a further aspect, the present invention relates to a method of identifying a compound which is capable of inhibiting the activity or function of a polypeptide defined in SEQ ID NO.:2 or a polypeptide comprising a polypeptide sequence encoded by SEQ ID NO.:1 (e.g., a transcribed portion, a translated portion, a fragment, substantially identical and even substantially complementary sequences). The method may comprise contacting the polypeptide with a putative compound an isolating or identifying a compound that is capable of specifically binding any one of the above-mentioned polypeptide. The compound may originate from a combinatorial library.

The method may also further comprise determining whether the activity or function of the polypeptide (e.g., a function attributed to SEQ ID NO.:2) is affected by the binding of the compound. Those compounds which are capable of binding to the polypeptide and which and/or which are capable of altering the function or activity of the polypeptide represents a desirable compound to be used in cancer therapy.

The method may also further comprise a step of determining the effect of the putative compound on the growth of a cancer cell such as an ovarian cancer cell.

The present invention also relates to an assay and method for identifying a nucleic acid sequence and/or protein involved in the growth or development of ovarian cancer. The assay and method may comprise silencing an endogenous gene of a cancer cell such as an ovarian cancer cell and providing the cell with a candidate nucleic acid (or protein). A candidate gene (or protein) positively involved in inducing cancer cell death (e.g., apoptosis) (e.g., ovarian cancer cell) may be identified by its ability to complement the silenced endogenous gene. For example, a candidate nucleic acid involved in ovarian cancer provided to a cell for which an endogenous gene has been silenced, may enable the cell to undergo apoptosis more so in the presence of an inducer of apoptosis.

Alternatively, an assay or method may comprise silencing an endogenous gene (gene expression) corresponding to the candidate nucleic acid or protein sequence to be evaluated and determining the effect of the candidate nucleic acid or protein on cancer growth (e.g., ovarian cancer cell growth). A sequence involved in the promotion or inhibition of cancer growth, development or malignancy may change the viability of the cell, may change the ability of the cell to grow or to form colonies, etc. The activity of a polypeptide may be impaired by targeting such polypeptide with an antibody molecule or any other type of compound. Again, such compound may be identified by screening combinatorial libraries, phage libraries, etc.

The present invention also provides a method for identifying an inhibitory compound (inhibitor, antagonist) able to impair the function (activity) or expression of a polypeptide described herein. The method may comprise, for example, contacting the (substantially purified or isolated) polypeptide or a cell expressing the polypeptide with a candidate compound and measuring the function (activity) or expression of the polypeptide. A reduction in the function or activity of the polypeptide (compared to the absence of the candidate compound) may thus positively identify a suitable inhibitory compound.

In accordance with the present invention, the impaired function or activity may be associated, for example, with a reduced ability of the polypeptide to reduce growth of an ovarian cancer cell or a reduced enzymatic activity or function attributed to the polypeptide.

The cell used to carry the screening test may not naturally (endogenously) express the polypeptide or analogs, or alternatively the expression of a naturally expressed polypeptide analog may be repressed.

As used herein the term “sequence identity” relates to (consecutive) nucleotides of a nucleotide sequence with reference to an original nucleotide sequence which when compared are the same or have a specified percentage of nucleotides which are the same. With respect to polypeptides, the term “sequence identity” relates to (consecutive) amino acids of a sequence with reference to an original sequence which when compared are the same or have a specified percentage of amino acids which are the same.

The identity may be compared over a region or over the total sequence of a nucleic acid sequence or amino acid sequence. Thus, “identity” may be compared, for example, over a region of 10, 19, 20 nucleotides or amino acids or more (and any number therebetween) and more preferably over a longer region or over the entire region of a polynucleotide or amino acid sequence described herein (e.g., SEQ ID NO.:1 or SEQ ID NO.:2). It is to be understood herein that gaps of non-identical nucleotides or amino acids may be found between identical nucleic acids regions (identical nucleotides). For example, a polynucleotide may have 100% identity with another polynucleotide over a portion thereof. However, when the entire sequence of both polynucleotides is compared, the two polynucleotides may have 50% of their overall (total) sequence identity to one another.

Percent identity may be determined, for example, with n algorithm GAP, BESTFIT, or FASTA in the Wisconsin Genetics Software Package Release 7.0, using default gap weights.

Polynucleotides or polypeptides of the present invention or portion thereof having from about 50 to about 100% and any range therebetween, or about 60 to about 100% or about 70 to about 100% or about 80 to about 100% or about 85% to about 100%, about 90% to about 100%, about 95% to about 100% sequence identity with an original polynucleotide or polypeptide are encompassed herewith. It is known by those of skill in the art, that a polynucleotide having from about 50% to 100% identity may function (e.g., anneal to a substantially complementary sequence) in a manner similar to an original polynucleotide and therefore may be used in replacement of an original polynucleotide. For example a polynucleotide (a nucleic acid sequence) may comprise or have from about 50% to about 100% identity with an original polynucleotide over a defined region and may still work as efficiently or sufficiently to achieve the present invention.

The term “substantially identical” used to define the polynucleotides of the present invention refers to polynucleotides which have, for example, from 50% to 100% sequence identity and any range therebetween but preferably at least 80%, at least 85%, at least 90%, at least 95% sequence identity and also include 100% identity with that of an original sequence (including sequences 100% identical over the entire length of the polynucleotide sequence).

“Substantially identical” polynucleotide sequences may be identified by providing a probe of about 10 to about 25, or more or about 10 to about 20 nucleotides long (or longer) based on the sequence of SEQ ID NO.:1 (more particularly, a transcribed and/or translated portion of SEQ ID NO.:1) and complementary sequence thereof and hybridizing a library of polynucleotide (e.g., cDNA or else) originating from another species, tissue, cell, individual etc. A polynucleotide that hybridizes under highly stringent conditions (e.g., 6×SCC, 65° C.) to the probe may be isolated and identified using methods known in the art. A sequence “substantially identical” includes for example, an isolated allelic variant, an isolated splice variant, an isolated non-human ortholog, a modified NSEQ etc.

As used herein the terms “sequence complementarity” refers to (consecutive) nucleotides of a nucleotide sequence that are complementary to a reference (original) nucleotide sequence. The complementarity may be compared over a region or over the total sequence of a nucleic acid sequence.

Polynucleotides of the present invention or portion thereof having from about 50 to about 100%, or about 60 to about 100% or about 70 to about 100% or about 80 to about 100% or about 85%, about 90%, about 95% to about 100% sequence complementarity with an original polynucleotide are thus encompassed herewith. It is known by those of skill in the art, that a polynucleotide having from about 50% to 100% complementarity with an original sequence may anneal to that sequence in a manner sufficient to carry out the present invention (e.g., inhibit expression of the original polynucleotide).

The term “substantially complementary” used to define the polynucleotides of the present invention refers to polynucleotides which have, for example, from 50% to 100% sequence complementarity and any range therebetween but preferably at least 80%, at least 85%, at least 90%, at least 95% sequence complementarity and also include 100% complementarity with that of an original sequence (including sequences 100% complementarity over the entire length of the polynucleotide sequence).

As used herein the term “polynucleotide” generally refers to any polyribonucleotide or polydeoxyribo-nucleotide, which may be unmodified RNA or DNA, or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus “polynucleotide” embraces chemically, enzymatically or metabolically modified forms of polynucleotides as typically found or not in nature, as well as the chemical forms of DNA and RNA characteristic of viruses and cells. “Polynucleotide” includes but is not limited to linear and end-closed molecules. “Polynucleotide” also embraces relatively short polynucleotides, often referred to as oligonucleotides.

“Polypeptides” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins. As described above, polypeptides may contain amino acids other than the 20 gene-encoded amino acids.

As used herein the term “polypeptide analog” or “analog” relates to mutants, chimeras, fusions, a polypeptide comprising at least one amino acid deletion, a polypeptide comprising at least one amino acid insertion or addition, a polypeptide comprising at least one amino acid substitutions, and any other type of modifications made relative to a given polypeptide.

An “analog” is thus to be understood herein as a molecule having a biological activity and/or chemical structure similar to that of a polypeptide described herein or having a defined level of amino acid identity. An “analog” may have sequence similarity or identity with that of an original sequence or a portion of an original sequence and may also have a modification of its structure as discussed herein. For example, an “analog” may have at least 80% or 85%, 90% or 95% sequence similarity or identity with an original sequence or a portion of an original sequence. An “analog” may also have, for example; at least 70% or even 50% sequence similarity or identity with an original sequence or a portion of an original sequence and may function in a suitable manner.

A “derivative” is to be understood herein as a polypeptide originating from an original sequence or from a portion of an original sequence and which may comprise one or more modification; for example, one or more modification in the amino acid sequence (e.g., an amino acid addition, deletion, insertion, substitution etc.), one or more modification in the backbone or side-chain of one or more amino acid, or an addition of a group or another molecule to one or more amino acids (side-chains or backbone). Biologically active derivatives of the carrier described herein are encompassed by the present invention. Also, an “derivative” may have, for example, at least 50%, 70%, 80%, 90% sequence similarity or identity to an original sequence with a combination of one or more modification in a backbone or side-chain of an amino acid, or an addition of a group or another molecule, etc.

As used herein the term “biologically active” refers to an analog which retains some or all of the biological activity of the original polypeptide, i.e., to have some of the activity or function associated with the polypeptide described herein, or to be able to promote or inhibit the growth ovarian cancer.

Therefore, any polypeptide having a modification compared to an original polypeptide that does not destroy significantly a desired activity, function or immunogenicity is encompassed herein. It is well known in the art, that a number of modifications may be made to the polypeptides of the present invention without deleteriously affecting their biological activity. These modifications may, on the other hand, keep or increase the biological activity of the original polypeptide or may optimize one or more of the particularity (e.g. stability, bioavailability, etc.) of the polypeptides of the present invention which, in some instance might be desirable. Polypeptides of the present invention may comprise for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino- or carboxy-terminus. It will be appreciated that the same type of modification may be present in the same or varying degrees at several sites in a given polypeptide. Also, a given polypeptide may contain many types of modifications. It is to be understood herein that more than one modification to the polypeptides described herein are encompassed by the present invention to the extent that the biological activity is similar to the original (parent) polypeptide.

As discussed above, polypeptide modification may comprise, for example, amino acid insertion, deletion and substitution (i.e., replacement), either conservative or non-conservative (e.g., D-amino acids, desamino acids) in the polypeptide sequence where such changes do not substantially alter the overall biological activity of the polypeptide.

Example of substitutions may be those, which are conservative (i.e., wherein a residue is replaced by another of the same general type or group) or when wanted, non-conservative (i.e., wherein a residue is replaced by an amino acid of another type). In addition, a non-naturally occurring amino acid may substitute for a naturally occurring amino acid (i.e., non-naturally occurring conservative amino acid substitution or a non-naturally occurring non-conservative amino acid substitution).

As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same type or group as that of the amino acid to be replaced. Thus, in some cases, the basic amino acids Lys, Arg and H is may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

It should be further noted that if the polypeptides are made synthetically, substitutions by amino acids, which are not naturally encoded by DNA (non-naturally occurring or unnatural amino acid) may also be made.

A non-naturally occurring amino acid is to be understood herein as an amino acid that is not naturally produced or found in a mammal. A non-naturally occurring amino acid comprises a D-amino acid, an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide. Non-naturally occurring amino acids (residues) include also the omega amino acids of the formula NH₂(CH₂)ₙCOOH wherein n is 2-6, neutral nonpolar amino acids, such as sarcosine, t-butyl alanine, t-butyl glycine, N-methyl isoleucine, norleucine, etc. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that analogs may be generated by substitutional mutagenesis and retain the biological activity of the polypeptides of the present invention. These analogs have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in Table A. If such substitutions result in a change not desired, then other type of substitutions, denominated “exemplary substitutions” in Table A, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:

(1) hydrophobic: norleucine, methionine (Met), Alanine (Ala), Valine (Val), Leucine (Leu), Isoleucine (Ile)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro); and aromatic: Tryptophan (Trp), Tyrosine (Tyr), Phenylalanine (Phe)

Non-conservative substitutions will entail exchanging a member of one of these classes for another.

TABLE A
Examplary amino acid substitution

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example, with respect to a percentage (%) of identity of from about 80 to 100%, it is to be understood as specifically incorporating herein each and every individual %, as well as sub-range, such as for example 80%, 81%, 84.78%, 93%, 99% etc. with respect to a length of “about 10 to about 25” it is to be understood as specifically incorporating each and every individual number such as for example 10, 11, 12, 13, 14, 15 up to and including 25; and similarly with respect to other parameters such as, concentrations, elements, etc.

Other objects, features, advantages, and aspects of the present invention will become apparent to those skilled in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

In the appended drawings:

FIG. 1 is a picture of the macroarray hybridization results showing the differential expression data for STAR selected ovarian cancer-related human SEQ. ID. NO. 1. The STAR dsDNA clone representing SEQ. ID. NO.1 was labeled with ³²P and hybridized to the macroarray. The hybridization results obtained confirm its upregulation in several of the malignant ovarian cancer samples (A-F 2 and A-G 3-4) compared to LMP samples (A-F 1). Significant expression of this sequence was also evident in the breast cancer cell line, MCF7 (B-C 5); Macroarrays were prepared using RAMP amplified RNA from six human LMP samples (A-F 1) and twenty malignant ovarian tumor samples (Table B) (A-F 2 and A-G 3-4), and 30 different normal human tissues (adrenal (A7), breast (B7), jejunum (C7), trachea (D7), liver (E7), placenta (F7), aorta (G7), brain (H7), lung (A8), adrenal cortex (B8), esophagus (C8), colon (D8), ovary (E8), kidney (F8), prostate (G8), thymus (H8), skeletal muscle (A9), vena cava (B9), stomach (C9), small intestine (D9), heart (E9), fallopian tube (F9), spleen (G9), bladder (H9), cervix (A10), pancreas (B10), ileum (C10), duodenum (D10), thyroid (E10) and testicle (F10)). Also included on the RNA macroarray were breast cancer cell lines (MDA (A5), MCF7 (B5) and MCF7+ estradiol (C5)) and LCM microdissected prostate normal epithelium (A-C 6) and prostate cancer (D-F 6), prostate cancer cell line, LNCap (G6) and LNCap+ androgen (H6). In these figures, the probe labeling reaction was also spiked with a dsDNA sequence for Arabidopsis, which hybridizes to the same sequence spotted on the macroarray (M) in order to serve as a control for the labeling reaction.

FIG. 2 is a picture of RT-PCR data showing the differential expression data for STAR selected SEQ ID NO.:1. To further demonstrate that the STAR SEQ. ID. NOs. selected after macroarray analysis were upregulated in malignant ovarian cancer samples compared to LMPs and normal ovarian samples, semi-quantitative RT-PCR was performed for 25 cycles using HotStarTaq polymerase according to the supplier instructions (Qiagen). Furthermore, these results serve to demonstrate the utility of these sequences as potential diagnostic, prognostic or theranostic markers for ovarian cancer. A specific primer pair was used for SEQ ID NO.:1. The differential expression results obtained for SEQ ID NO.:1 is shown in FIG. 2. As indicated by the expected PCR amplicon product for SEQ ID NO.:1, there is a clear tendency towards increased expression of the mRNAs corresponding to SEQ ID NO.:1 in clear cell carcinoma (Lanes 8-9), late stage endometrioid (Lane 12) and different stages of malignant serous (Lanes 15-17) compared to normal (Lane 1), benign (Lanes 2-3) and LMPs (Lanes 4-7) ovarian samples. These results confirm the upregulation of the gene expression for SEQ ID NO.:1 in the different stages of malignant ovarian cancer as was observed using the macroarrays;

FIG. 3A shows the expression profiling analyses using semi-quantitative RT-PCR reactions carried out to measure the level of KAAG1 mRNA expression in RNA samples derived from greater than 20 ovarian tumors, benign (low malignancy potential) tumors, ovarian cancer cell lines, and 30 normal tissues. The control panels show GAPDH expression, a house-keeping gene used to compare the amount of starting material in each RT-PCR reaction;

FIG. 3B shows semi-quantitative RT-PCR experiments demonstrating that KAAG1 mRNA is expressed in ovarian cancer cell lines, in particular those that are derived from ascites;

FIG. 3C shows a diagram illustrating the ability of ovarian cancer cell lines to form 3D structures called spheroids. The left panels show the cells grown in medium lacking serum whereas 5% serum stimulated the formation of the spheroid structures;

FIG. 3D shows semi-quantitative RT-PCR experiments demonstrating that the KAAG1 mRNA is highly induced during the formation of spheroids in ovarian cancer cell lines;

FIG. 4A shows a diagram illustrating the wound or scratch assay, a cell-based assay that is a measurement of a cell line's ability to migrate into a denuded area over a pre-determined period of time. TOV-21G cells harboring KAAG1 shRNAs display a reduced capacity to fill in the denuded area;

FIG. 4B shows an illustration of the clonogenic assay, also known as a colony survival assay. It measured the survival of diluted cells over a period of several days. TOV-21G cells harboring KAAG1 shRNAs display reduced survival;

FIG. 5 is a picture of RT-PCR data showing the differential expression data for the STAR selected ovarian cancer-related human SEQ ID NO.:1 in RNA samples derived from the NCl-60 panel of cancer cell lines. A primer pair, OGS 1067 (GAGGGGCATCAATCACACCGAGAA; SEQ. ID. NO. 45) and OGS 1068 (CCCCACCGCCCACCCATTTAGG; SEQ. ID. NO. 46) for SEQ ID NO.:1 was used to perform RT-PCR. As indicated by the expected PCR amplicon, increased expression of SEQ ID NO.:1 mRNA was evident in ovarian, renal, lung, colon, breast cancer, and melanoma but weakly in CNS cancer and leukemia;

FIG. 6A shows a polyacrylamide gel that was stained with Coomassie Blue and contains a sample (10 μg) of purified Fc-KAAG1 fusion protein that was produced in transiently transfected 293E cells;

FIG. 6B shows the results of an ELISA of one of the 96-well plates containing individual monoclonal antibodies selected from Omniclonal library #3 containing anti-KAAG1 Fabs. The results showed that 48 (highlighted in grey) of the Fabs interacted very efficiently with KAAG1. The wells indicated by bold numbers contained the exemplary monoclonals 3D3, 3G10, and 3C4;

FIG. 7A shows a polyacrylamide gel that was stained with Coomassie Blue and contains a sample (10 μg) of purified Fc-KAAG1 fusion protein (lane 1), a truncated mutant of KAAG1 spanning amino acids 1-60 (lane 2), and another truncated mutant of KAAG1 spanning amino acids 1-35 (lane 3) that were produced in transiently transfected 293E cells. All proteins were Fc fusion proteins;

FIG. 7B is a scheme that illustrates the truncated mutants of KAAG1 that were generated for the epitope mapping studies;

FIG. 7C shows a drawing that describes the results from ELISA analyses to map the epitopes that are bound by the anti-KAAG1 antibodies contained in Omniclonal library #3. The results showed that the majority of monoclonals interact with central region of KAAG1 and that certain antibodies bound to the amino- or carboxyl-termini of KAAG1;

FIG. 8 presents a scheme that illustrates the steps involved to convert the mouse Fabs into IgG1 mouse-human chimeric mAbs;

FIG. 9 shows drawings that compare the binding of the mouse anti-KAAG1 Fabs with the binding of the corresponding IgG1 chimeric monoclonal antibodies for exemplary antibodies 3D3, 3G10, and 3C4. The results indicate that the relative binding of the Fab variable regions was maintained when transferred to a full human IgG1 scaffold;

FIG. 10 shows depictions of spheroid formation experiments using TOV-21G and OV-90 ovarian cancer cell lines in the presence of chimeric IgG1 anti-KAAG1 monoclonal antibodies. Loosely packed structures are indicative of less invasive cancer cell lines. The results show spheroids treated with the exemplary anti-KAAG1 antibodies 3D3, 3G10, or 3C4;

FIG. 11A shows a scan of a tissue microarray containing approximately 70 biopsy samples obtained from ovarian tumor patients. The samples were blotted with the 3D3 anti-KAAG1 antibody and showed that the vast majority of ovarian tumors expressed very high level of KAAG1 antigen;

FIG. 11B a higher magnification picture from the tissue microarray experiment. The arrows show the membrane localization of KAAG1 at the apical surface of the epithelial layer of cells in serous ovarian tumors;

FIG. 11C illustrates other immunohistochemical studies that demonstrate that KAAG1 is highly expressed in all ovarian cancer types. The histotypes shown are serous, mucinous and endometrioid;

FIG. 12 An IgG1 antibody that targets KAAG1 can efficiently mediate ADCC activity in vitro. PBMNCs (AllCells, LLC, Emoryville, Calif.) were incubated with 3D3 for 30 min and mixed with either OVCAR-3 or WIL2-S cells at a ratio of 1:25. The cells were incubated for 4 h at 37 C and cell lysis was determined by measuring LDH levels in the medium. Cell cytotoxicity was calculated as follows: % cytotoxicity=(experimental−effector spontaneous−target spontaneous)×100/(target maximum−target spontaneous);

FIG. 13 Anti-KAAG1 mAbs prevent the spread of TOV-112D ovarian tumors in vivo. 1×10⁶ cells were implanted in the peritoneal cavity of SCID mice in a volume of 200 μL. Treatment with either PBS or antibodies diluted in PBS was performed 2 days later at a dose of 25 mg/kg qwk. The mice were sacrificed as soon as the tumors were detected by palpation of the abdomen. The number of tumors were scored visually (B) and the data in panel A is expressed as the average number of tumors/mouse±SE;

FIG. 14 shows immunohistochemistry performed with an anti-KAAG1 antibody on human skin tumor tissue microarrays (Pantomics Inc., Richmond, Calif.) of several sections isolated from squamous cell carcinomas and melanomas;

FIG. 15 illustrates spheroid formation of melanoma cell lines (A375 and SK-MEL5) and of renal cell carcinoma cell lines (A498 and 786-O) in the presence or absence of the chimeric 3D3 antibody;

FIG. 16A represents graphs illustrating the binding of increasing concentrations of the 3C4, 3D3 and 3G10 antibodies to cell lines (OV-90, TOV-21G and SKOV-3) fixed under condition that do not permeate the cells;

FIG. 16B is a graph illustrating the results of flow cytometry performed on SKOV-3 cell line with the 3D3 antibody;

FIG. 17A is a graph illustrating the binding of increasing concentration of the humanized 3D3 antibody in comparison with the chimeric 3D3 antibody to recombinant KAAG1;

FIG. 17B is a table summarizing the kinetics parameters of the humanized 3D3 antibody, the chimeric 3D3 antibody as well as hybrid antibodies encompassing permutations of the light and heavy chains of the chimeric or humanized antibody;

FIG. 17C illustrates spheroid formation of SKOV-3 ovarian cancer cells in the presence of the humanized 3D3 antibody, chimeric 3D3 antibody or in the presence of a buffer or a control IgG;

FIG. 18 shows the expression of the KAAG1 antigen on the surface of the ovarian cancer cell line OVCAR-3 as measured by immunofluorescence. The cells were stained with the chimeric 3D3 antibody followed by visualization with a fluorescently labelled secondary antibody. The cell surface expression was confirmed with the co-localization of an known surface protein, E-cadherin;

FIG. 19 represents the detection of the KAAG1 antigen on the surface of SKOV-3 cells by flow cytometry. The fluorescent signal decreases with time when the cells were incubated at 37 C, which suggests that the KAAG1/antibody complex was internalized during the incubation; and

FIG. 20 shows that the KAAG1 antigen that was detected by the chimeric 3D3 antibody gets internalized soon after the complex is formed. Punctate peri-nuclear staining was observed at 30 minutes of incubation at 37 C which was consistent with the complex following an endosomal pathway.

### DESCRIPTION OF ILLUSTRATIVE EMBODIMENTS

The applicant employed a carefully planned strategy to identify and isolate genetic sequences involved in ovarian cancer. The process involved the following steps: 1) preparation of highly representative cDNA libraries using mRNA isolated from LMPs and malignant ovarian cancer samples of human origin; 2) isolation of sequences upregulated in the malignant ovarian cancer samples; 3) identification and characterization of upregulated sequences; 4) selection of upregulated sequences for tissue specificity; 5) determination of knock-down effects on ovarian cancer cell line proliferation and migration; and 6) determination of the expression pattern of each upregulated sequence in samples derived from nine different cancer types. The results discussed in this disclosure demonstrate the advantage of targeting ovarian cancer-related genes that are highly specific to this differentiated cell type compared to normal tissues and provide a more efficient screening method when studying the genetic basis of diseases and disorders. Polynucleotide and/or polypeptide sequences that are known but have not had a role assigned to them until the present disclosure have also been isolated and shown to have a critical role in ovarian cancer cell line proliferation and migration. Finally, novel polynucleotide and/or polypeptide sequences have been identified that play a role as well.

The present invention is illustrated in further details below in a non-limiting fashion.

### A—Material and Methods

Commercially available reagents referred to in the present disclosure were used according to supplier's instructions unless otherwise indicated. Throughout the present disclosure certain starting materials were prepared as follows:

B—Preparation of LMP and malignant ovarian cancer cells

LMP and malignant ovarian tumor samples were selected based on histopathology to identify the respective stage and grade (Table B). LMP was chosen instead of normal ovarian tissue to avoid genes that associated with proliferation due to ovulation. Also very few cells would have been recovered and stromal cells would have been a major contaminant. LMP and serous (most common) ovarian tumors represent the extremes of tumorigenicity, differentiation and invasion. Once the sample were selected, total RNA was extracted with Trizol™ (InVitrogen, Grand Island, N.Y.) after the tissues were homogenized. The quality of the RNA was assessed using a 2100 Bioanalyzer (Agilent Technologies, Palo Alto, Calif.)

TABLE B
shows the pathologies including grade and stage of the different ovarian
cancer samples used on the macroarrays.

| | | | | | Position |
|---|---|---|---|---|---|
| MF | | | | | on |
| Code | | | | | Macro- |
| No. | Pathologies | Symbol | Stage | Grade | array |
| 15 | Borderline serous | B | 1b | B | A1 |
| 16 | Borderline serous | B | 2a | B | B1 |
| 17 | Borderline/carcinoma serous | B/CS | 3c | 1 | F1 |
| 18 | Borderline serous | B | 3c | B | C1 |
| 19 | Borderline serous | B | 1b | B | D1 |
| 20 | Borderline serous | B | 1a | B | E1 |
| 42 | Carcinoma serous of the | CSS | 3a | 3 | A4 |
| | surface | | | | |
| 22 | Carcinoma serous | CS | 1b | 3 | A2 |
| 30 | Carcinoma serous | CS | 2c | 3 | E2 |
| 23 | Carcinoma serous | CS | 3c | 3 | F2 |
| 25 | Carcinoma serous | CS | 3c | 3 | B2 |
| 26 | Carcinoma serous | CS | 3c | 3 | A3 |
| 27 | Carcinoma serous | CS | 3c | 3 | C2 |
| 28 | Carcinoma serous | CS | 3c | 3 | D2 |
| 43 | Carcinoma serous | CS | 3c | 3 | B4 |
| 45 | Carcinoma serous | CS | 3c | 3 | D4 |
| 49 | Carcinoma serous | CS | 3c | 2 | F4 |
| 41 | Carcinoma endometrioide | CE | 3b | 3 | G3 |
| 40 | Carcinoma endometrioide | CE | 3c | 3 | F3 |
| 44 | Carcinoma endometrioide | CE | 3c | 3 | C4 |
| 39 | Carcinoma endometrioide | CE | 3c | 2 | E3 |
| 50 | Carcinoma endometrioide | CE | 1c | 1 | G4 |
| 46 | Carcinoma endometrioide | CE | 1a | 2 | E4 |
| 34 | Clear cell carcinoma | CCC | 3c | 2 | B3 |
| 38 | Clear cell carcinoma | CCC | 3c | 3 | D3 |
| 37 | Clear cell carcinoma | CCC | 1c | 2 | C3 |

C—Method of Isolating Differentially Expressed mRNA

Key to the discovery of differentially expressed sequences unique to malignant ovarian cancer is the use of the applicant's patented STAR technology (Subtractive Transcription-based Amplification of mRNA; U.S. Pat. No. 5,712,127 Malek et al., 1998). Based on this procedure, mRNA isolated from malignant ovarian tumor sample is used to prepare “tester RNA”, which is hybridized to complementary single-stranded “driver DNA” prepared from mRNA from LMP sample and only the un-hybridized “tester RNA” is recovered, and used to create cloned cDNA libraries, termed “subtracted libraries”. Thus, the “subtracted libraries” are enriched for differentially expressed sequences inclusive of rare and novel mRNAs often missed by micro-array hybridization analysis. These rare and novel mRNA are thought to be representative of important gene targets for the development of better diagnostic and therapeutic strategies.

The clones contained in the enriched “subtracted libraries” are identified by DNA sequence analysis and their potential function assessed by acquiring information available in public databases (NCBI and GeneCard). The non-redundant clones are then used to prepare DNA micro-arrays, which are used to quantify their relative differential expression patterns by hybridization to fluorescent cDNA probes. Two classes of cDNA probes may be used, those which are generated from either RNA transcripts prepared from the same subtracted libraries (subtracted probes) or from mRNA isolated from different ovarian LMP and malignant samples (standard probes). The use of subtracted probes provides increased sensitivity for detecting the low abundance mRNA sequences that are preserved and enriched by STAR. Furthermore, the specificity of the differentially expressed sequences to malignant ovarian cancer is measured by hybridizing radio-labeled probes prepared from each selected sequence to macroarrays containing RNA from different LMP and malignant ovarian cancer samples and different normal human tissues.

A major challenge in gene expression profiling is the limited quantities of RNA available for molecular analysis. The amount of RNA isolated from many human specimens (needle aspiration, laser capture micro-dissection (LCM) samples and transfected cultured cells) is often insufficient for preparing: 1) conventional tester and driver materials for STAR; 2) standard cDNA probes for DNA micro-array analysis; 3)

RNA macroarrays for testing the specificity of expression; 4) Northern blots and; 5) full-length cDNA clones for further biological validation and characterization etc. Thus, the applicant has developed a proprietary technology called RAMP (RNA Amplification Procedure) (U.S. patent application Ser. No. 11/000,958 published under No. US 2005/0153333A1 on Jul. 14, 2005 and entitled “Selective Terminal Tagging of Nucleic Acids”), which linearly amplifies the mRNA contained in total RNA samples yielding microgram quantities of amplified RNA sufficient for the various analytical applications. The RAMP RNA produced is largely full-length mRNA-like sequences as a result of the proprietary method for adding a terminal sequence tag to the 3′-ends of single-stranded cDNA molecules, for use in linear transcription amplification. Greater than 99.5% of the sequences amplified in RAMP reactions show <2-fold variability and thus, RAMP provides unbiased RNA samples in quantities sufficient to enable the discovery of the unique mRNA sequences involved in ovarian cancer.

### D—Preparation of Human Malignant Ovarian Cancer Subtracted Library

Total RNA from five human ovarian LMP samples (MF-15, -16, -18, -19 and -20) (Table B) and five malignant ovarian cancer samples (MF-22, -25, -27, -28 and -30) (Table B) (CHUM, Montreal, QC) were prepared as described above. Following a slight modification of the teachings of Malek et al., 1998 (U.S. Pat. No. 5,712,127) i.e., preparation of the cDNA libraries on the paramagnetic beads as described below), 1 μg of total RNA from each sample were used to prepare highly representative cDNA libraries on streptavidin-coated paramagnetic beads (InVitrogen, Grand Island, N.Y.) for preparing tester and driver materials. In each case, first-strand cDNA was synthesized using an oligo dT₁₁ primer with 3′ locking nucleotides (e.g., A, G or C), a 5′-biotin moiety and containing a Not I recognition site (OGS 364: SEQ. ID. NO. 27) Next, second-strand cDNA synthesis was performed according to the manufacturer's procedure for double-stranded cDNA synthesis (Invitrogen, Burlington, ON) and the resulting double-stranded cDNA ligated to linkers containing an Asc I recognition site (New England Biolabs, Pickering, ON). The double-stranded cDNAs were then digested with Asc I and Not I restriction enzymes (New England Biolabs, Pickering, ON), purified from the excess linkers using the cDNA fractionation column from Invitrogen (Burlington, ON) as specified by the manufacturer. Each sample was equally divided and ligated separately to specialized oligonucleotide promoter tags, TAG1 (OGS 594 and 595: SEQ. ID. NO: 28 and SEQ. ID. NO:29) and TAG2 (OGS458 and 459: SEQ. ID. NO:30 and SEQ. ID. NO:31) used for preparing tester and driver materials, respectively. Thereafter, each ligated cDNA was purified by capturing on the streptavidin beads as described by the supplier (InVitrogen, Grand Island, N.Y.), and transcribed in vitro with T7 RNA polymerase (Ambion, Austin, Tex.).

Next, in order to prepare 3′-represented tester and driver libraries, a 10-μg aliquot of each of the in vitro synthesized RNA was converted to double-stranded cDNA by performing first-strand cDNA synthesis as described above followed by primer-directed (primer OGS 494 (SEQ. ID. NO:32) for TAG1 and primer OGS 302 (SEQ. ID. NO:33) for TAG2) second-strand DNA synthesis using Advantage-2 Taq polymerase (BD Biosciences Clontech, Mississauga, ON). The double-stranded cDNA was purified using Qiaquick columns and quantified at A₂₆₀ₙₘ. Thereafter, 6x 1-μg aliquots of each double-stranded cDNA was digested individually with one of the following 4-base recognition restriction enzymes Rsa I, Sau3A1, Mse I, Msp I, HinPI I and Bsh 1236I (MBI Fermentas, Burlington, ON), yielding up to six possible 3′-fragments for each RNA species contained in the cDNA library. Following digestion, the restriction enzymes were inactivated with phenol and the set of six reactions pooled. The restriction enzymes sites were then blunted with T4 DNA polymerase and ligated to linkers containing an Asc I recognition site. Each linker-adapted pooled DNA sample was digested with Asc I and Not I restriction enzymes, desalted and ligated to specialized oligonucleotide promoter tags, TAG1 (OGS 594 and 595) for the original TAG1-derived materials to generate tester RNA and TAG2-related OGS 621 and 622 (SEQ. ID. NO:34 and SEQ. ID. NO:35) with only the promoter sequence for the original TAG2-derived materials for generating driver DNA. The promoter-ligated materials were purified using the streptavidin beads, which were then transcribed in vitro with either T7 RNA polymerase (Ambion, Austin, Tex.), purified and quantified at A₂₆₀ₙₘ. The resulting TAG1 3′-represented RNA was used directly as “tester RNA” whereas, the TAG2 3′-represented RNA was used to synthesize first-strand cDNA, which then served as single-stranded “driver DNA”. Each “driver DNA” reaction was treated with RNase A and RNase H to remove the RNA, phenol extracted and purified before use. An equivalent amount of each driver RNA for the five LMP samples were pooled before synthesis of the single-stranded driver DNA.

The following 3′-represented libraries were prepared:

Tester 1 (MF-22)—human malignant ovarian cancer donor 1

Tester 2 (MF-25)—human malignant ovarian cancer donor 2

Tester 3 (MF-27)—human malignant ovarian cancer donor 3

Tester 4 (MF-28)—human malignant ovarian cancer donor 4

Tester 5 (MF-30)—human malignant ovarian cancer donor 5

Driver 1 (MF-15)—human ovarian LMP donor 1

Driver 2 (MF-16)—human ovarian LMP donor 2

Driver 3 (MF-18)—human ovarian LMP donor 3

Driver 4 (MF-19)—human ovarian LMP donor 4

Driver 5 (MF-20)—human ovarian LMP donor 5

Each tester RNA sample was subtracted following the teachings of U.S. Pat. No. 5,712,127 with the pooled driver DNA (MF-15, -16, -18, -19 and -20) in a ratio of 1:100 for 2-rounds following the teachings of Malek et al., 1998 (U.S. Pat. No. 5,712,127). Additionally, control reactions containing tester RNA and no driver DNA, and tester RNA plus driver DNA but no RNase H were prepared. The tester RNA remaining in each reaction after subtraction was converted to double-stranded DNA, and a volume of 5% removed and amplified in a standard PCR reaction for 30-cycles for analytical purposes. The remaining 95% of only the tester-driver plus RNase H subtracted samples after 2-rounds were amplified for 4-cycles in PCR, digested with Asc I and Not I restriction enzymes, and one half ligated into the pCATRMAN (SEQ. ID. NO:36) plasmid vector and the other half, into the p20 (SEQ. ID. NO.:37) plasmid vector. The ligated materials were transformed into E. coli DH10B and individual clones contained in the pCATRMAN libraries were picked for further analysis (DNA sequencing and hybridization) whereas, clones contained in each p20 library were pooled for use as subtracted probes. Each 4-cycles amplified cloned subtracted library contained between 15,000 and 25,000 colonies. Additionally, in order to prepare subtracted cDNA probes, reciprocal subtraction for g-rounds was performed using instead, the pooled driver RNA as “tester” and each of the malignant tester RNA as “driver”. The materials remaining after subtraction for each were similarly amplified for 4-cycles in PCR, digested with Asc I and Not I restriction enzymes, and one half ligated into the p20 plasmid vector.

The following cloned subtracted libraries were prepared:

SL123—Tester 1 (MF-22) minus Pooled Driver (MF-15, -16, -18, -19 and -20)
SL124—Tester 2 (MF-25) minus Pooled Driver (MF-15, -16, -18, -19 and -20)
SL125—Tester 3 (MF-27) minus Pooled Driver (MF-15, -16, -18, -19 and -20)
SL126—Tester 4 (MF-28) minus Pooled Driver (MF-15, -16, -18, -19 and -20)
SL127—Tester 5 (MF-30) minus Pooled Driver (MF-15, -16, -18, -19 and -20)
SL133—Pooled Driver (MF-15, -16, -18, -19 and -20) minus Tester 1 (MF-22)
SL134—Pooled Driver (MF-15, -16, -18, -19 and -20) minus Tester 2 (MF-25)
SL135—Pooled Driver (MF-15, -16, -18, -19 and -20) minus Tester 3 (MF-27)
SL136—Pooled Driver (MF-15, -16, -18, -19 and -20) minus Tester 4 (MF-28)
SL137—Pooled Driver (MF-15, -16, -18, -19 and -20) minus Tester 5 (MF-30)

A 5-μL aliquot of the 30-cycles PCR amplified subtracted and non-subtracted materials were visualized on a 1.5% agarose gel containing ethidium bromide and then transferred to Hybond N+ (Amersham Biosciences, Piscataway, N.J.) nylon membrane for Southern blot analysis. Using radiolabeled probes specific for GAPDH (glyceraldehyde-3-phosphate dehydrogenase; Accession #M32599.1) and β-actin (Accession #X00351), which are typically non-differentially expressed house-keeping genes, it was evident that there was subtraction of both GAPDH and β-actin (data not shown). Yet, at the same time, a probe specific for CCNE1 (Accession # NM_{—}001238, a gene known to be upregulated in malignant ovarian cancer, indicated that it was not subtracted (data not shown). Based on these results, it was anticipated that the subtracted libraries would be enriched for differentially expressed upregulated sequences.

### E—Sequence Identification and Annotation of Clones Contained in the Subtracted Libraries:

Approximately ˜5300 individual colonies contained in the pCATRMAN subtracted libraries (SL123 to SL127) described above were randomly picked using a Qbot (Genetix Inc., Boston, Mass.) into 60 μL of autoclaved water. Then, 42 μL of each was used in a 100 μL standard PCR reaction containing oligonucleotide primers, OGS 1 and OGS 142 and amplified for 40-cycles (94° C. for 10 minutes, 40× (94° C. for 40 seconds, 55° C. for 30 seconds and 72° C. for 2 minutes) followed by 72° C. for 7 minutes) in 96-wells microtitre plates using HotStart™ Taq polymerase (Qiagen, Mississauga, ON). The completed PCR reactions were desalted using the 96-well filter plates (Corning) and the amplicons recovered in 100 μL 10 mM Tris (pH 8.0). A 5-μL aliquot of each PCR reaction was visualized on a 1.5% agarose gel containing ethidium bromide and only those reactions containing a single amplified product were selected for DNA sequence analysis using standard DNA sequencing performed on an ABI 3100 instrument (Applied Biosystems, Foster City, Calif.). Each DNA sequence obtained was given a Sequence Identification Number and entered into a database for subsequent tracking and annotation.

Each sequence was selected for BLAST analysis of public databases (e.g. NCBI). Absent from these sequences were the standard housekeeping genes (GAPDH, actin, most ribosomal proteins etc.), which was a good indication that the subtracted library was depleted of at least the relatively abundant non-differentially expressed sequences.

Once sequencing and annotation of the selected clones were completed, the next step involved identifying those sequences that were actually upregulated in the malignant ovarian cancer samples compared to the LMP samples.

### F—Hybridization Analysis for Identifying Upregulated Sequences

The PCR amplicons representing the annotated sequences from the pCATRMAN libraries described above were used to prepare DNA microarrays. The purified PCR amplicons contained in 70 μL of the PCR reactions prepared in the previous section was lyophilized and each reconstituted in 20 μL of spotting solution comprising 3×SSC and 0.1% sarkosyl. DNA micro-arrays of each amplicon in triplicate were then prepared using CMT-GAP2 slides (Corning, Corning, N.Y.) and the GMS 417 spotter (Affymetrix, Santa Clara, Calif.).

The DNA micro-arrays were then hybridized with either standard or subtracted cy3 and cy5 labelled cDNA probes as recommended by the supplier (Amersham Biosciences, Piscataway, N.J.). The standard cDNA probes were synthesized using RAMP amplified RNA prepared from the different human ovarian LMP and malignant samples. It is well known to the skilled artisan that standard cDNA probes only provide limited sensitivity of detection and consequently, low abundance sequences contained in the cDNA probes are usually missed. Thus, the hybridization analysis was also performed using cy3 and cy5 labelled subtracted cDNA probes prepared from in vitro transcribed RNA generated from subtracted libraries (SLP123 to SLP127 and SLP133 to SLP137) cloned into the p20 plasmid vector and represent the different tester and driver materials. These subtracted libraries may be enriched for low abundance sequences as a result of following the teachings of Malek et al., 1998 (U.S. Pat. No. 5,712,127), and therefore, may provide increased detection sensitivity.

All hybridization reactions were performed using the dye-swap procedure as recommended by the supplier (Amersham Biosciences, Piscataway, N.J.) and approximately 750 putatively differentially expressed upregulated (>2-fold) sequences were selected for further analysis.

### G—Determining Malignant Ovarian Cancer Specificity of the Differentially Expressed Sequences Identified:

The differentially expressed sequences identified in Section F for the different human malignant ovarian cancer subtracted libraries (SL123 to SL127) were tested for specificity by hybridization to nylon membrane-based macroarrays. The macroarrays were prepared using RAMP amplified RNA from 6 LMP and 20 malignant human ovarian samples, and 30 normal human tissues (adrenal, liver, lung, ovary, skeletal muscle, heart, cervix, thyroid, breast, placenta, adrenal cortex, kidney, vena cava, fallopian tube, pancreas, testicle, jejunum, aorta, esophagus, prostate, stomach, spleen, ileum, trachea, brain, colon, thymus, small intestine, bladder and duodenum) purchased commercially (Ambion, Austin, Tex.). In addition, RAMP RNA prepared from breast cancer cell lines, MDA and MCF7, prostate cancer cell line, LNCap, and a normal and prostate cancer LCM microdissected sample. Because of the limited quantities of mRNA available for many of these samples, it was necessary to first amplify the mRNA using the RAMP methodology. Each amplified RNA sample was reconstituted to a final concentration of 250 ng/μL in 3×SSC and 0.1% sarkosyl in a 96-well microtitre plate and 1 μL spotted onto Hybond N+ nylon membranes using the specialized MULTI-PRINT™ apparatus (VP Scientific, San Diego, Calif.), air dried and UV-cross linked. Of the ˜750 different sequences selected from SL123 to SL127 for macroarray analysis, only 250 sequences were individually radiolabeled with α-³²P-dCTP using the random priming procedure recommended by the supplier (Amersham, Piscataway, N.J.) and used as probes on the macroarrays thus far. Hybridization and washing steps were performed following standard procedures well known to those skilled in the art.

Occasionally, the results obtained from the macroarray methodology were inconclusive. For example, probing the membranes with certain STAR clones resulted in patterns where all the RNA samples appeared to express equal levels of the message or in patterns where there was no signal. This suggested that not all STAR clones were useful tools to verify the expression of their respective genes. To circumvent this problem, RT-PCR was used to determine the specificity of expression. Using the same RAMP RNA samples that were spotted on the macroarrays, 500 μg of RNA was converted to single-stranded cDNA with Thermoscript RT (Invitrogen, Burlington, ON) as described by the manufacturer. The cDNA reaction was diluted so that 1/200 of the reaction was used for each PCR experiment. After trial PCR reactions with gene-specific primers designed against each SEQ. ID NOs. to be tested, the linear range of the reaction was determined and applied to all samples, PCR was conducted in 96-well plates using Hot-Start Taq Polymerase from Qiagen (Mississauga, ON) in a DNA Engine Tetrad from MJ Research. Half of the reaction mixture was loaded on a 1.2% agarose/ethidium bromide gel and the amplicons visualized with UV light.

Of the 250 sequences tested, approximately 55% were found to be upregulated in many of the malignant samples compared to the LMPs. However, many of these sequences were also readily detected in a majority of the different normal human tissues. Based on these results, those sequences that were detected in many of the other human tissues at significantly elevated levels were eliminated. Consequently, only 49 sequences, which appeared to be upregulated and highly malignant ovarian cancer-specific, were selected for biological validation studies. This subset of 49 sequences include some genes previously reported in the literature to be upregulated in ovarian cancer but without demonstration of their relative expression in normal tissues. The macroarray data for FOLR1 was included to exemplify the hybridization pattern and specificity of a gene that is already known to be involved in the development of ovarian cancer (data not shown).

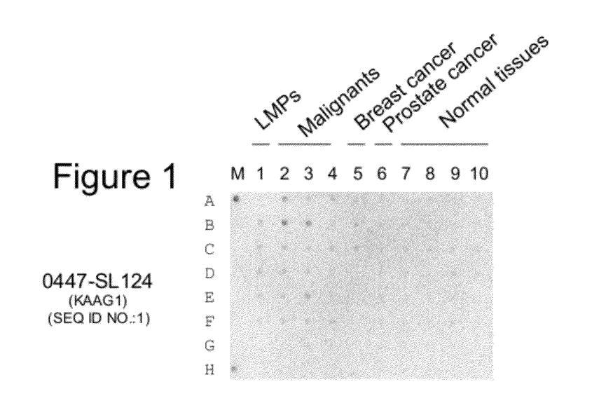

Amongst the 50 selected sequences, 27 were associated with genes having functional annotation 15 were associated with genes with no functional annotation and 8 were novel sequences (genomic hits). The identification of gene products involved in regulating the development of ovarian cancer has thus led to the discovery of highly specific, including novel targets, for the development of new therapeutic strategies for ovarian cancer management. FIG. 1 shows the macroarray hybridization signal patterns and RT-PCR amplification data for the malignant ovarian cancer and normal human tissues relative to LMPs for SEQ ID NO.:1 isolated and selected for biological validation.

The method described herein may be used to preferentially identify a sequence which is upregulated in malignant ovarian cancer cell compared to a cell from a low malignancy potential ovarian cancer and/or compared to a normal cell.

In accordance with the present invention, a sequence may be further selected based on a reduced, lowered or substantially absent expression in a subset of other normal cell (e.g., a normal ovarian cell) or tissue, therefore representing a candidate sequence specifically involved in ovarian cancer.

The method may also further comprise a step of determining the complete sequence of the nucleotide sequence and may also comprise determining the coding sequence of the nucleotide sequence.

A sequence may also be selected for its specificity to other types of tumor cells, thus identifying a sequence having a more generalized involvement in the development of cancer. These types of sequence may therefore represent desirable candidates having a more universal utility in the treatment and/or detection of cancer.

The present invention also relates in a further aspect, to the isolated differentially expressed sequence (polynucleotide and polypeptide) identified by the method of the present invention.

### EXAMPLES

### Example 1

### SEQ. ID. NO:1

SEQ ID NO.:1 is one of the sequences identified using the method described above. The candidate protein encoded by the isolated SEQ. ID. NO:1 is a previously identified gene that encodes a protein, kidney associated antigen 1 (KAAG1), which has no known function (NCBI Unigene # Gene Symbol Hs.512599; Acession No. NM_{—}000077; open reading frame; 213-683 encoding SEQ ID NO.:2 (KAAG1)). We have demonstrated that expression of this gene is markedly upregulated in malignant ovarian cancer samples compared to ovarian LMP samples and a majority of normal human tissues (FIG. 1), which have not been previously reported. Thus, it is believed that expression of the gene may be required for, or involved in ovarian cancer tumorigenesis.

We have also demonstrated that Folate receptor 1 (adult) (FOLR1) is markedly upregulated in malignant ovarian cancer samples compared to ovarian LMP samples and a majority of normal human tissues (data not shown). The potential role of FOLR1 in ovarian cancer therapeutics has been previously documented (Leamon and Low, 2001 and Jhaveri et al., 2006, U.S. Pat. No. 7,030,236). By way of example of the FOLR1 gene target, similar genes described herein with upregulation in malignant ovarian tumors and limited or no expression in a majority of normal tissues may also serve as potential therapeutic targets for ovarian cancer.

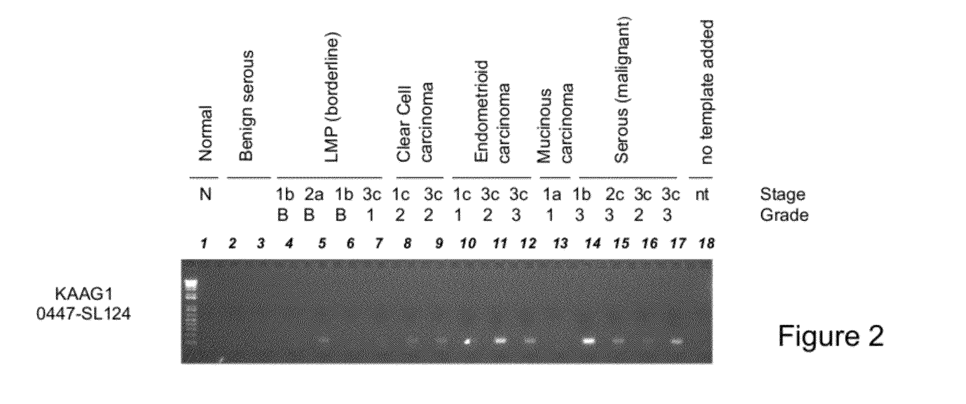

To further demonstrate that SEQ ID NO.:1 was upregulated in malignant ovarian cancer samples compared to LMPs and normal ovarian samples, semi-quantitative RT-PCR was performed for 25 cycles using HotStarTaq polymerase according to the supplier instructions (Qiagen). A specific primer pair was used for SEQ ID NO.:1. The differential expression results obtained for SEQ ID NO.:1 is shown in FIG. 2. As indicated by the expected PCR amplicon product for SEQ ID NO.:1, there is a clear tendency towards increased expression of the mRNAs corresponding to SEQ ID NO.:1 in clear cell carcinoma (Lanes 8-9), late stage endometrioid (Lane 12) and different stages of malignant serous (Lanes 15-17) compared to normal (Lane 1), benign (Lanes 2-3) and LMPs (Lanes 4-7) ovarian samples. These results confirm the upregulation of the gene expression for SEQ ID NO.:1 in the different stages of malignant ovarian cancer as was observed using the macroarrays. Furthermore, these results serve to demonstrate the utility of these sequences as potential diagnostic, prognostic or theranostic markers for ovarian cancer.

### Example 2

### Expression of the KAAG1 Gene in Ovarian Tumors and Ovarian Cancer Cell Line

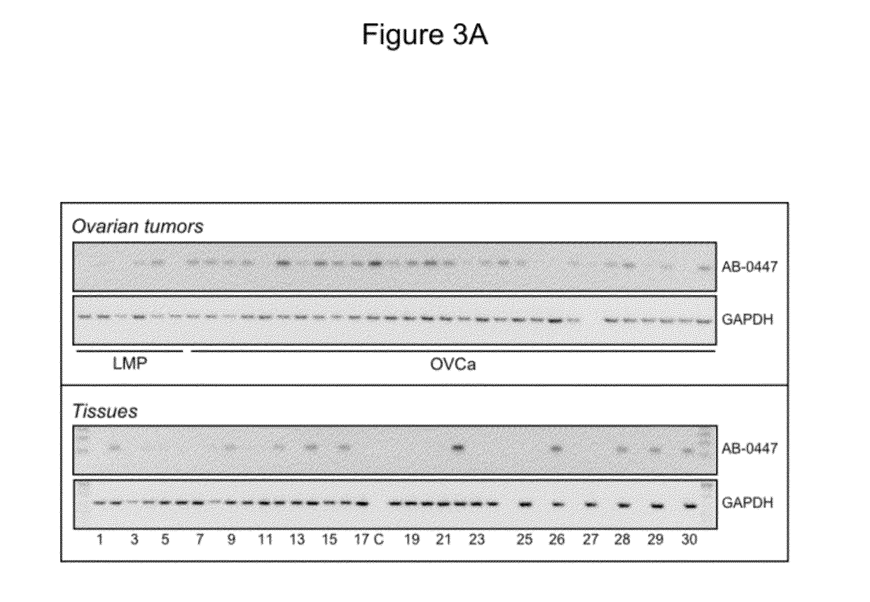

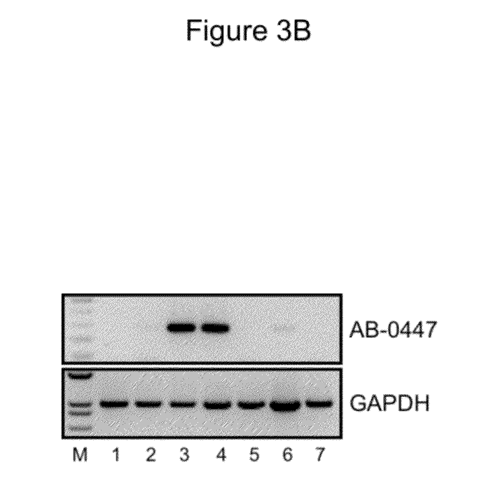

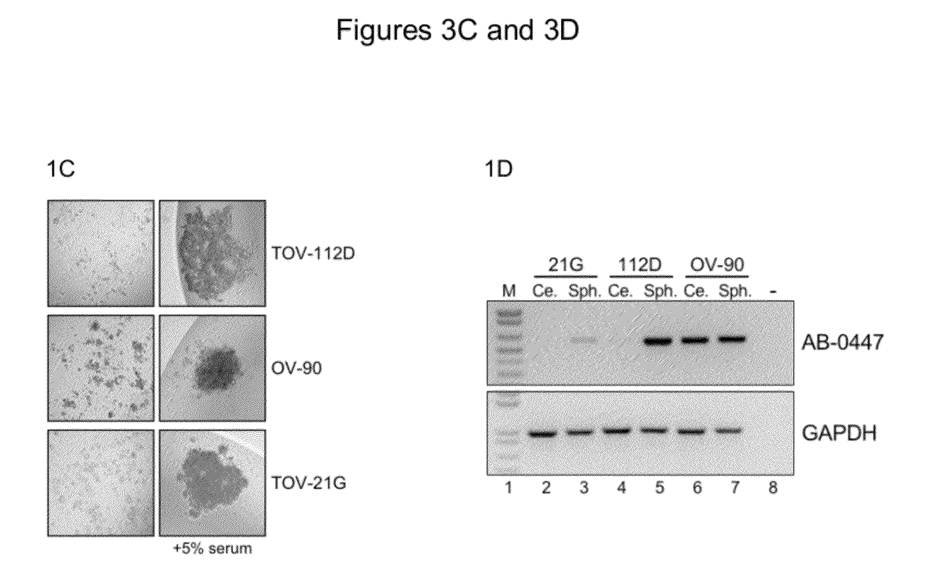

PCR analysis was performed to verify the percentage of ovarian tumors that express the mRNA encoding KAAG1 (indicated as AB-0447 in the Figure). The results showed that the KAAG1 gene is expressed in greater than 85% of ovarian tumors from all stages of the disease and 100% of late stage tumors. The expression of KAAG1 is lower or undetectable in LMP samples (see FIG. 3A). For each sample, 1 μg of amplified RNA was reverse transcribed with random hexamers using Thermoscript RT (Invitrogen). The cDNA was diluted and 1/200th of the reaction was used as template for each PCR reaction with gene-specific primers as indicated. The primers used to amplify the KAAG1 mRNA contained the sequences shown in SEQ ID NOS:40 and 41. PCR reactions were carried out in 96-well plates and half of the 25 μl reaction was electrophoresed on a 1% agarose gel. The gels were visualized and photographed with a gel documentation system (BioRad). The upper panel of FIG. 3A shows the results from 6 LMP samples (LMP) and 22 ovarian tumor and 6 ovarian cell line (last 6 lanes on the right, OVCa) samples. The lower panel of FIG. 3 shows the RNA samples from 30 normal tissues that were tested as indicated.

KAAG1 expression was weakly detected in a few normal tissues whereas the mRNA was evident in the fallopian tube and the pancreas (see FIG. 3A). The amount of total RNA used in these reactions was controlled with parallel PCR amplifications of glyceraldehyde-3-phosphate dehydrogenase (GAPDH), a housekeeping gene, and the results showed that equivalent starting material was present in each sample (see FIG. 3A). The primers used to amplify the GAPDH gene contained the sequences shown in SEQ ID NOs: 42 and 43. Thus, the expression of the KAAG1 gene fulfills an important selection criteria: it is over-expressed in a large proportion of ovarian tumors and its expression is low or absent in most normal tissues. These data suggest that ovarian tumors may be specifically targeted with high affinity monoclonal antibodies against KAAG1.

Early stage cancer or tumors tend to be made up of cells that are in a high state of differentiation but as the tumor progresses to a more aggressive and invasive state, the cancer cells become increasingly undifferentiated. There are needs to identify factors that contribute to this transition and exploit these proteins as targets for the development of therapeutics. Several ovarian cancer cell lines are available that were derived from primary tumors and serve as excellent models for the functional studies. The expression of KAAG1 was examined in these cell lines. Four lines termed TOV-21G, TOV-112D, TOV-1946, and TOV-2223G were established from primary tumors whereas OV-90 and OV-1946 are cell lines derived from cells contained in ascites fluid of patients with advanced ovarian cancer. Total RNA from cells established from primary tumors (see in FIG. 3B, lanes 1, TOV-21G; 2, TOV-112D; 5, TOV-1946; 6, TOV-2223G) and cells established from ascitic cells (lanes 3, OV-90; 4, OV-1946) was converted to cDNA with reverse transcriptase and used as template in PCR reactions with KAAG1-specific primers (SEQ ID NOS:45 and 46). As a negative control, the reaction was carried out with total RNA from normal ovary. Equal amounts of starting material were utilized as evidenced by parallel PCR reactions with GAPDH (SEQ ID NOS:42 and 43). A sample of the PCR reaction was electrophoresed on an agarose gel and visualized with ethidium bromide. As shown in FIG. 3B, KAAG1 was detectable but weakly expressed in the cell lines from the primary tumors and PCR reactions performed at a higher number of cycles revealed the KAAG1 transcript in all four of these cell lines. Conversely, both cell lines established from the ascitic fluid cells exhibited high level of the KAAG1 transcript. The increased expression in cells from the ascitic fluid suggests that the environment of the cells influences the regulation of the KAAG1 gene.

Ascitic cells are associated with advanced disease and the pattern of expression disclosed in FIG. 3B implies that increased KAAG1 levels are associated with anchorage-independent growth. This question was addressed by culturing the cells in hanging droplets, a condition that prevents the cells from adhering to the petri dish, as is the case when they are grown as monolayers. These so called three-dimensional cultures allow the cells to associate and the formation of spheroids is observed (see FIG. 3C). Spheroids were cultures as follows: TOV-112D, OV-90, or TOV-21G cells (4 000 in 15 μl) were incubated for 4 days in medium in the absence (left panels, FIG. 3C) or presence of 5% FBS (right panels, FIG. 3C, +5% serum). The magnification of the image was set to 100×. These spheroids have been extensively characterized and exhibit many of the properties found in primary tumors including morphological and functional properties as well as the molecular signature as measured by microarray-based expression profiling.

Total RNA was isolated from spheroid preparations and RT-PCR was performed as described for FIG. 3A. TOV-21G, TOV-112D, OV-90 cells were seeded as described in the legend for FIG. 3C under conditions to produce spheroids. After 4 days, total RNA was isolated and used to perform RT-PCR reactions with KAAG1-specific primers (SEQ ID NOS:45 and 46). PCR reactions were electrophoresed on agarose gels. Conducting parallel reactions to amplify GAPDH (SEQ ID NOS:42 and 43) demonstrated that equal amounts of starting material were present in each sample. The following acronyms are used in FIG. 3D: Ce., cells grown as monolayers; Sph., cells grown as spheroids. Strikingly, KAAG1 expression was up-regulated when TOV-21G and TOV-112D were grown as spheroids (see FIG. 3D). In the case of the OV-90 cells, the level of expression of the KAAG1 gene was unchanged and remained very high. Presumably, the level of expression attained in the cell lines derived from the ascitic fluid, as exemplified by the OV-90 cells and the OV-1946 cells (see FIG. 3A) has reached a maximum.

These results correlated with the previous data showing high expression in cell lines derived from ascitic fluid and confirm that expression of KAAG1 is influenced by the microenvironment of the cancer cells. Additionally, the up-regulation of KAAG1 transcription that was observed in spheroids implies that high levels of KAAG1 are present in malignant ovarian cancer.

### Example 3

RNA interference is a recently discovered gene regulation mechanism that involves the sequence-specific decrease in a gene's expression by targeting the mRNA for degradation and although originally described in plants, it has been discovered across many animal kingdoms from protozoans and invertebrates to higher eukaryotes (reviewed in Agrawal et al., 2003). In physiological settings, the mechanism of RNA interference is triggered by the presence of double-stranded RNA molecules that are cleaved by an RNAse III-like protein active in cells, called Dicer, which releases the 21-23 bp siRNAs. The siRNA, in a homology-driven manner, complexes into a RNA-protein amalgamation termed RISC(RNA-induced silencing complex) in the presence of mRNA to cause degradation resulting in attenuation of that mRNA's expression (Agrawal et al., 2003).

Current approaches to studying the function of genes, such as gene knockout mice and dominant negatives, are often inefficient, and generally expensive, and time-consuming. RNA interference is proving to be a method of choice for the analysis of a large number of genes in a quick and relatively inexpensive manner. Although transfection of synthetic siRNAs is an efficient method, the effects are often transient at best (Hannon G. J., 2002). Delivery of plasmids expressing short hairpin RNAs by stable transfection has been successful in allowing for the analysis of RNA interference in longer-term studies (Brummelkamp et al., 2002; Elbashir et al., 2001).

### Determination of Knockdown Effects on the Proliferation of Ovarian Cancer Cell Lines

In order to determine which ovarian cancer-specific genes participate in the proliferation of ovarian cancer cells, an assay was developed using stably transfected cell lines that contain attenuated (i.e., knocked down) levels of the specific gene being investigated. Two human ovarian cancer cell lines derived from chemotherapy-naïve patients were utilized that have been previously characterized in terms of their morphology, tumorigenicity, and global expression profiles. In addition, these analyses revealed that these cell lines were excellent models for in vivo behavior of ovarian tumors in humans (Provencher et al., 2000 and Samouelian et al., 2004). These cell lines are designated TOV-21G and TOV-112D.

The design and subcloning of individual shRNA expression cassettes and the procedure utilized for the characterisation of each nucleotide sequence is described below. Selection of polynucleotides were chosen based on their upregulation in ovarian tumors and the selective nature of their expression in these tumors compared to other tissues as described above. The design of shRNA sequences was performed using web-based software that is freely available to those skilled in the art (Qiagen for example). These chosen sequences, usually 19-mers, were included in two complementary oligonucleotides that form the template for the shRNAs, i.e. the 19-nt sense sequence, a 9-nt linker region (loop), the 19-nt antisense sequence followed by a 5-6 poly-T tract for termination of the RNA polymerase III. Appropriate restriction sites were inserted at the ends of these oligonucleotides to facilitate proper positioning of the inserts so that the transcriptional start point is at a precise location downstream of the hU6 promoter. The plasmid utilized in all RNA interference studies, pSilencer 2.0 (SEQ. ID. NO. 51), was purchase from a commercial supplier (Ambion, Austin, Tex.). For each sequence selected, at least two different shRNA expression vectors were constructed to increase the chance of observing RNA interference.

TOV-21G or TOV-112D cells were seeded in 6-well plates in OSE (Samouelian et al., 2004) containing 10% fetal bovine serum at a density of 600 000 cells/well, allowed to plate overnight and transfected with 1 μg of pSil-shRNA plasmid using the Fugene 6 reagent (Roche, Laval, QC). After 16 h of incubation, fresh medium was added containing 2 μg/ml puromycin (Sigma, St. Louis, Mo.) to select for stable transfectants. Control cells were transfected with a control pSil that contains a scrambled shRNA sequence that displays homology to no known human gene. After approximately 4-5 days, pools and/or individual clones of cells were isolated and expanded for further analyses. The effectiveness of attenuation was verified in all shRNA cells lines. Total RNA was prepared by standard methods using Trizol™ reagent from cells grown in 6-well plates and expression of the target gene was determined by RT-PCR using gene-specific primers. First strand cDNA was generated using Thermoscript (Invitrogen, Burlington, ON) and semi-quantitative PCR was performed by standard methods (Qiagen, Mississauga, ON). 100% expression levels for a given gene was assigned to those found in the cell lines transfected with the control pSil plasmid (sh-scr).

Results from the attenuation of two candidate genes, indicate that the shRNAs that were expressed in the TOV-21G stable transfectants were successful in attenuating the expression of their target genes. As a control for equal quantities of RNA in all reactions, the expression of glyceraldehyde-3-phosphate dehydrogenase was monitored and found to be expressed at equal levels in all samples used (data not shown).

The proliferative ability of each shRNA-expressing cell line was determined and compared to cells expressing the scrambled shRNA (control). Cell number was determined spectrophotometrically by MTT assay at 570 nm (Mosmann, 1983). After selection of stably shRNA expressing pools and expansion of the lines, 5 000 cells/well of each cell lines was plated in 48-well plates in triplicate and incubated for 4 days under standard growth conditions. Experiments were typically repeated at least three times to confirm the results observed. The cell number after 4 days in the control cell line expressing the scrambled shRNA was arbitrarily set to 100%. TOV-21G cell lines containing shRNA against three of the tested sequences exhibited less than 50% proliferation for at least one shRNA compared to the control cell line (not shown). The proliferation of TOV-21G cell lines containing shRNA against two other sequences was not affected to the same extent but significant inhibition of growth was still observed nevertheless. These results indicate that attenuation of genes identified by the methods also used to identify SEQ ID NO.1 causes retardation in the growth of this ovarian cancer cell line. Several of these shRNA expression vectors were also transfected into the TOV-112D cell line and similar results were obtained (data not shown). This suggested that these genes are important for proliferation of ovarian cancer cells.

The gene encoding the folate receptor 1 was also attenuated in TOV-21G cells, and marked growth inhibition was observed in the presence of the shRNAs. This gives credibility to the approach used to validate the genes presented in this patent and substantiated their functional importance in the proliferation of ovarian cancer cells.

As a means of complementing the growth inhibition data that was generated with the stable TOV-21G cell lines, a colony survival assay was used to determine the requirement of the selected genes in the survival of the cancer cells. The ‘colony formation assay’ or ‘clonogenic assay’ is a classical test to evaluate cell growth after treatment. The assay is widespread in oncological research areas where it is used to test the proliferating power of cancer cell lines after radiation and/or treatment with anticancer agents. It was expected that the results obtained when analyzing the genes that were functionally important in ovarian cancer would correlate between the growth inhibition study and the colony survival assay.

TOV-21G cells were seeded in 12-well plates at a density of 50 000 cells/well and transfected 24 h later with 1 μg of pSil-shRNA vector, the same plasmids used in the previous assay. The next day, fresh medium was applied containing 2 μg/ml puromycin and the selection of the cells was carried out for 3 days. The cells were washed and fresh medium without puromycin was added and growth continued for another 5 days. To visualize the remaining colonies, the cells were washed in PBS and fixed and stained simultaneously in 1% crystal violet/10% ethanol in PBS for 15 minutes at room temperature. Following extensive washing in PBS, the dried plates were scanned for photographic analysis.

Therefore, these results implied that a phenotypic manifestation was indicative of important genes that are functionally required in ovarian cancer cells and suggest that inhibition of the proteins they encode could be serve as important targets to develop new anticancer drugs. SEQ ID NOs.: 44 and 45 (siRNA against SEQ ID NO.: 1) were not successful at blocking proliferation in this experimental setting.

### Role for KAAG1 in the Survival of Ovarian Cancer Cells

With the demonstration that KAAG1 expression is regulated in ovarian cancer cells, the function of this gene in these cells was further examined. To address this question, in vitro assays were conducted to determine if this protein plays a role in cancer cell proliferation, migration, and/or survival. RNAi was used to knock down the expression of the endogenous KAAG1 gene in the TOV-21G ovarian cancer cell line. The design of two separate short-hairpin RNA (shRNA) sequences was performed using web-based software that is freely available to those skilled in the art (Qiagen for example). These chosen sequences, usually 19-mers, were included in two complementary oligonucleotides that form the template for the shRNAs, i.e. the 19-nt sense sequence, a 9-nt linker region (loop), the 19-nt antisense sequence followed by a 5-6 poly-T tract for termination of the RNA polymerase III. The sequences of the 19-mers that were used to knock down the expression of KAAG1 are shown in SEQ ID NOS: 44 and 45. Appropriate restriction sites were inserted at the ends of these oligonucleotides to facilitate proper positioning of the inserts so that the transcriptional start point is at a precise location downstream of the hU6 promoter. The plasmid utilized in all RNA interference studies, pSilencer 2.0 (SEQ ID NO.:46), was purchase from a commercial supplier (Ambion, Austin, Tex.). Two different shRNA expression vectors were constructed to increase the chance of observing RNAi effects and the specificity of phenotypic observations. TOV-21G cells were seeded in 6-well plates and transfected 24 h later with 1 μg of pSil-shRNA vector. Sh.1 and sh.2 were used to designate 2 different shRNA sequences targeting the KAAG1 gene. Stable transfectants were selected for 5-7 days, expanded, and grown to confluence. All of the following in vitro cell-based assays were performed using these stably transfected cell lines that contain shRNAs specific for KAAG1.

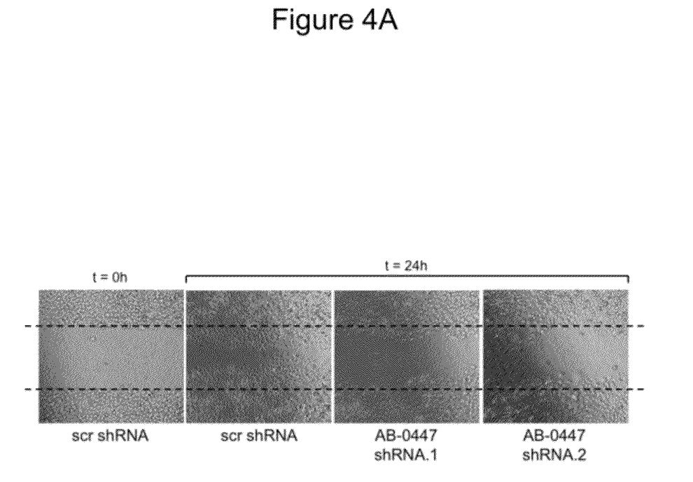

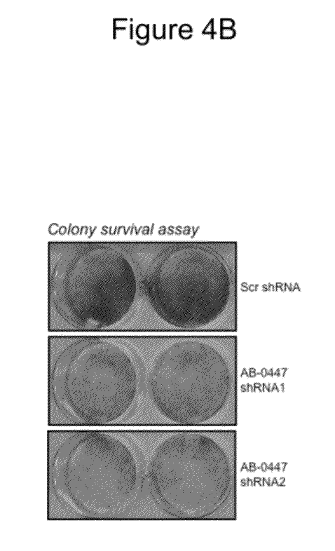

The migration or mobility of the cells was measured in a standard cell motility assay. This scratch assay, as it is called, measures the speed at which cells fill a denuded area in a confluent monolayer. As illustrated in FIG. 4A, TOV-21G cells containing the scrambled shRNA filled up the wound almost completely after 24 h compared to the control untreated cells (compare middle-left panel with left panel). By contrast, the ability of TOV-21G cells expressing KAAG1 shRNAs to fill the denuded area was greatly reduced. In fact, the number of cells that filled the denuded area in the presence of the KAAG1 shRNA cells more closely resembled the number of cells at time Oh (compare the left panel with the right panels).

To examine the longer-term effects of reduced expression of KAAG1 in ovarian cancer cells, the cells were extensively diluted and cultured for 10 days in a colony survival assay. TOV-21G cells were seeded in 12-well plates at a density of 50 000 cells/well and transfected 24 h later with 1 μg of pSil-shRNA vector. Sh-1 and sh-2 are used to designate 2 different shRNA sequences targeting the same gene. The next day, fresh medium was applied containing 2 μg/ml puromycin and the selection of the cells was carried out for 3 days. The cells were washed and fresh medium without puromycin was added and growth continued for another 5 days. To visualize the remaining colonies, the cells were washed in PBS and fixed and stained simultaneously in 1% crystal violet/10% ethanol in PBS for 15 minutes at room temperature. Following extensive washing in PBS, the dried plates were scanned for photographic analysis. A significant decrease in the survival of the cancer cell line was observed and a representative experiment is displayed in FIG. 4B. Identical results were obtained when the shRNAs were transfected into another ovarian cancer cell line, TOV-112D.

Thus, taken together, these results support an important role for KAAG1 in ovarian cancer cells. Furthermore, these results suggest that an antagonist of KAAG1 protein, such as a monoclonal antibody, would result in reduced invasiveness and decreased tumor survival.

### Example 4

### KAAG1 Involved in Other Oncology Indications

One skilled in the art will recognize that the sequences described in this invention have utilities in not only ovarian cancer, but these applications can also be expanded to other oncology indications where the genes are expressed. To address this, a PCR-based method was adapted to determine the expression pattern of all sequences described above in cancer cell lines isolated from nine types of cancer. The cancer types represented by the cell lines are leukemia, central nervous system, breast, colon, lung, melanoma, ovarian, prostate, and renal cancer (see Table C). These RNA samples were obtained from the Developmental Therapeutics Program at the NCl/NIH. Using the same RAMP RNA samples that amplified from the total RNA samples obtained from the NCl, 500 μg of RNA was converted to single-stranded cDNA with Thermoscript RT (Invitrogen, Burlington, ON) as described by the manufacturer. The cDNA reaction was diluted so that 1/200 of the reaction was used for each PCR experiment. After trial PCR reactions with gene-specific primers designed against each SEQ. ID NOs. to be tested, the linear range of the reaction was determined and applied to all samples, PCR was conducted in 96-well plates using Hot-Start Taq Polymerase from Qiagen (Mississauga, ON) in a DNA Engine Tetrad from MJ Research. Half of the reaction mixture was loaded on a 1.2% agarose/ethidium bromide gel and the amplicons visualized with UV light. To verify that equal quantities of RNA was used in each reaction, the level of RNA was monitored with GAPDH expression.

TABLE C
List of cancer cell lines from the NCI-60 panel

| Cell line | Cancer type |
|---|---|
| K-562 | leukemia |
| MOLT-4 | leukemia |
| CCRF-CEM | leukemia |
| RPMI-8226 | leukemia |
| HL-60(TB) | leukemia |
| SR | leukemia |
| SF-268 | CNS |
| SF-295 | CNS |
| SF-539 | CNS |
| SNB-19 | CNS |
| SNB-75 | CNS |
| U251 | CNS |
| BT-549 | breast |
| HS 578T | breast |
| MCF7 | breast |
| NCI/ADR-RES | breast |
| MDA-MB-231 | breast |
| MDA-MB-435 | breast |
| T-47D | breast |
| COLO 205 | colon |
| HCC-2998 | colon |
| HCT-116 | colon |
| HCT-15 | colon |
| HT29 | colon |
| KM12 | colon |
| SW-620 | colon |
| A549/ATCC | non-small cell lung |
| EKVX | non-small cell lung |
| HOP-62 | non-small cell lung |
| HOP-92 | non-small cell lung |
| NCI-H322M | non-small cell lung |
| NCI-H226 | non-small cell lung |
| NCI-H23 | non-small cell lung |
| NCI-H460 | non-small cell lung |
| NCI-H522 | non-small cell lung |
| LOX IMVI | melanoma |
| M14 | melanoma |
| MALME-3M | melanoma |
| SK-MEL-2 | melanoma |
| SK-MEL-28 | melanoma |
| SK-MEL-5 | melanoma |
| UACC-257 | melanoma |
| UACC-62 | melanoma |
| IGROV-1 | ovarian |
| OVCAR-3 | ovarian |
| OVCAR-4 | ovarian |
| OVCAR-5 | ovarian |
| OVCAR-8 | ovarian |
| SK-OV-3 | ovarian |
| DU-145 | prostate |
| PC-3 | prostate |
| 786-O | renal |
| A498 | renal |
| ACHN | renal |
| CAKI-1 | renal |
| RXF-393 | renal |
| SN-12C | renal |
| TK-10 | renal |
| UO-31 | renal |

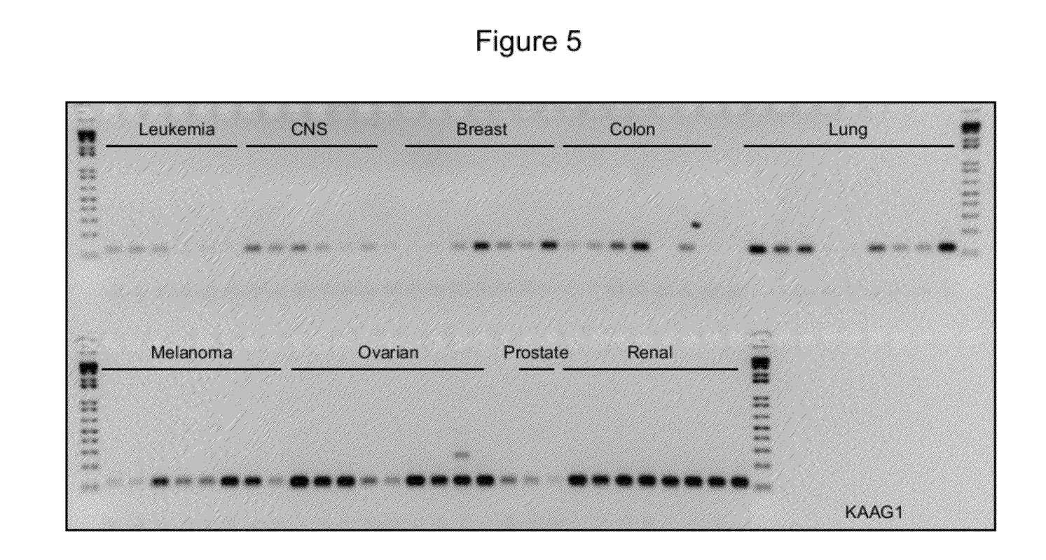

The NCl-60 panel includes 59 cell lines that are derived from tumors encompassing 9 human cancer types including leukemia, the central nervous system, breast, colon, lung, melanoma, ovarian, prostate, and renal. Complementary DNAs were prepared using random hexamers from RAMP amplified RNA from 59 human cancer cell lines (Table C). The cDNAs were quantified and used as templates for PCR with gene-specific primers using standard methods known to those skilled in the art. For each PCR result depicted in FIG. 5, equal amounts of template cDNA used in each PCR reaction was confirmed by reamplifying GAPDH with a specific primer pair, OGS 315 (TGAAGGTCGGAGTCAACGGATTTGGT; SEQ. ID. NO. 38) and OGS 316 (CATGTGGGCCATGAGGTCCACCAC; SEQ. ID. NO. 39) for this housekeeping gene.

Results of this experiment shows an increased expression of SEQ ID NO.:1 mRNA in ovarian cancer, renal cancer, lung cancer, colon cancer, breast cancer, and melanoma but weakly in CNS cancer and leukemia.

### Example 5

This example provides details pertaining to the family of monoclonal antibodies that bind to KAAG1.

The antibodies that bind KAAG1 were generated using the Biosite phage display technology. A detailed description of the technology and the methods for generating these antibodies can be found in the U.S. Pat. No. 6,057,098. Briefly, the technology utilizes stringent panning of phage libraries that display the antigen binding fragments (Fabs). After a several rounds of panning, a library, termed the Omniclonal, was obtained that was enriched for recombinant Fabs containing light and heavy chain variable regions that bound to KAAG1 with very high affinity and specificity. From this library, more precisely designated Omniclonal AL0003Z1, 96 individual recombinant monoclonal Fabs were prepared from E. coli and tested for KAAG1 binding.

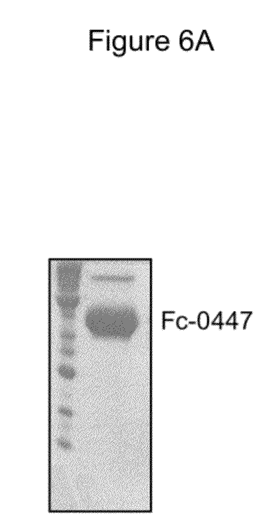

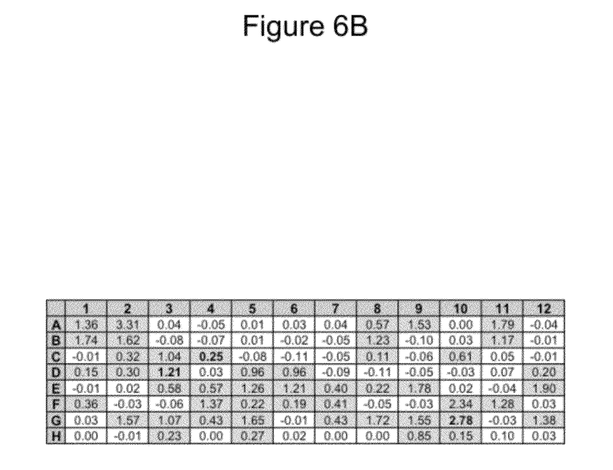

To measure the relative binding of each individual monoclonal antibody, recombinant human KAAG1 was produced in 293E cells using the large-scale transient transfection technology (Durocher et al., 2002; Durocher, 2004). The entire coding region of the KAAG1 cDNA was amplified by PCR using a forward primer that incorporated a BamHI restriction site (SEQ ID NO.:47) and a reverse primer that incorporated a HindIII restriction site (SEQ ID NO.:48). The resulting PCR product measured 276 base pairs and following digestion with BamHI and HindIII, the fragment was ligated into the expression vector pYD5 (SEQ ID NO.:49) that was similarly digested with the same restriction enzymes. The pYD5 expression plasmid contains the coding sequence for the human Fc domain that allows fusion proteins to be generated as well as the sequence encoding the IgG1 signal peptide to allow the secretion of the fusion protein into the culture medium. For each milliliter of cells, one microgram of the expression vector, called pYD5-0447, was transfected in 293E cells grown in suspension to a density of 1.5-2.0 million cells/ml. The transfection reagent used was polyethylenimine (PEI), (linear, MW 25,000, Cat #23966 Polysciences, Inc., Warrington, Pa.) which was included at a DNA:PEI ratio of 1:3. Growth of the cells was continued for 5 days after which the culture medium was harvested for purification of the recombinant Fc-KAAG1 fusion protein. The protein was purified using Protein-A agarose as instructed by the manufacturer (Sigma-Aldrich Canada Ltd., Oakville, ON). A representative polyacrylamide gel showing a sample of the purified Fc-KAAG1 (indicated as Fc-0447) is shown in FIG. 6A.

The 96-well master plate of monoclonal preparations contained different concentrations of purified anti-KAAG1 Fabs in each well. A second stock master plate was prepared by diluting the Fabs to a final concentration of 10 μg/ml from which all subsequent dilutions were performed for ELISA measurements. To carry out the binding of Fc-KAAG1 to the monoclonal preparations, the Fc-KAAG1 was biotinylated with NHS-biotin (Pierce, Rockford, Ill.) and 10 ng/well was coated in a streptavidin 96-well plate. One nanogram of each Fab monoclonal preparation was added to each well and incubated at room temperature for 30 minutes. Bound antibody was detected with HRP-conjugated mouse anti-kappa light chain antibody in the presence of TMB liquid substrate (Sigma-Aldrich Canada Ltd., Oakville, ON) and readings were conducted at 450 nm in microtiter plate reader. As shown in FIG. 6B, a total of 48 (highlighted in grey) monoclonal antibodies displayed significant binding in this assay (>0.1 arbitrary OD₄₅₀ units). The antibodies were purposely diluted to 1 ng/well to accentuate the binding of those antibodies with the most affinity for KAAG1. As a control, the antibodies did not bind to biotinylated Fc domain. These data also revealed that the binding of the antibodies varied from well to well indicating that they exhibited different affinities for KAAG1.

### Example 6

This example describes the epitope mapping studies to determine which region of KAAG1 the antibodies bind to.

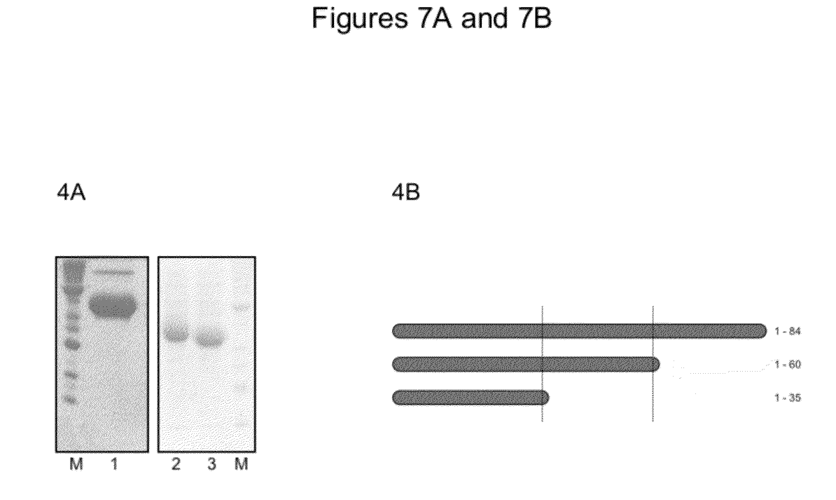

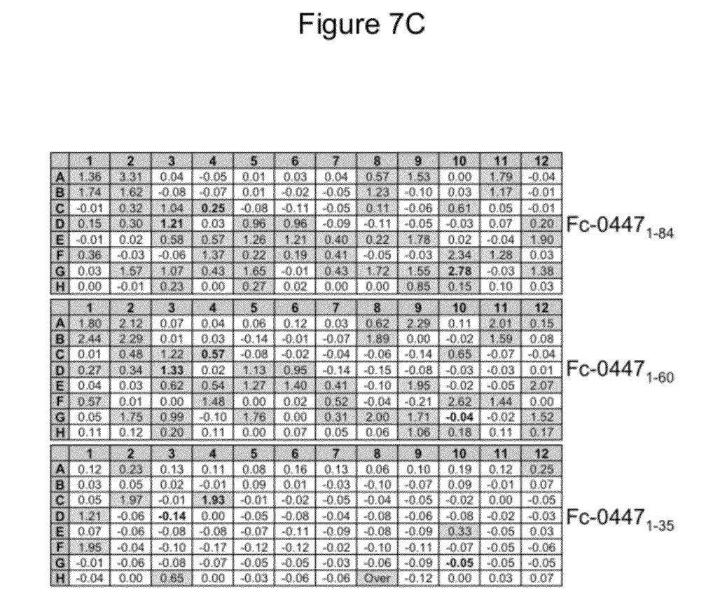

To further delineate the regions of KAAG1 that are bound by the monoclonal antibodies, truncated mutants of KAAG1 were expressed and used in the ELISA. As for the full length KAAG1, the truncated versions were amplified by PCR and ligated into BamHI/HindIII digested pYD5. The primers that were used combined the forward oligonucleotide with the sequence shown in SEQ ID NO.:47 with primers of SEQ ID NOS:50 and 51, to produce Fc-fused fragments that ended at amino acid number 60 and 35 of KAAG1, respectively. The expression of these mutants was conducted as was described above for the full length Fc-KAAG1 and purified with Protein-A agarose. A representative gel of the protein preparations that were used in the ELISA is shown in FIG. 7A and a schematic of the mutant proteins used for epitope mapping is depicted in FIG. 7B.

The results showed that the library was comprised of antibodies that could bind to each of the delineated KAAG1 regions. In particular, of the 48 mAbs that bound to KAAG1 in the first ELISA, nine (wells A2, A12, C2, C4, D1, E10, F1, H3, and H8) were found to interact with the first 35 amino acids of KAAG1 whereas five (D12, E8, F5, G10, and H5) were found to interact with the last 25 amino acids of KAAG1. Thus, the remaining 34 antibodies interacted with a region of KAAG1 spanned by amino acids 36-59. These results were in agreement with the sequence analysis of 24 representative light and heavy chain variable regions. Indeed, alignment of these sequences revealed that the antibodies clustered into three groups based on the percentage identity in their respective CDRs. Antibodies contained in each cluster all interacted with the same region of KAAG1.

Therefore, based on the relative binding affinity of the mAb, differential epitope interaction characteristics, and the differences in variable domain sequences, three antibodies from the plate described in Example 5 were selected for further analysis as exemplary anti-KAAG1 monoclonal antibodies.

### Example 7

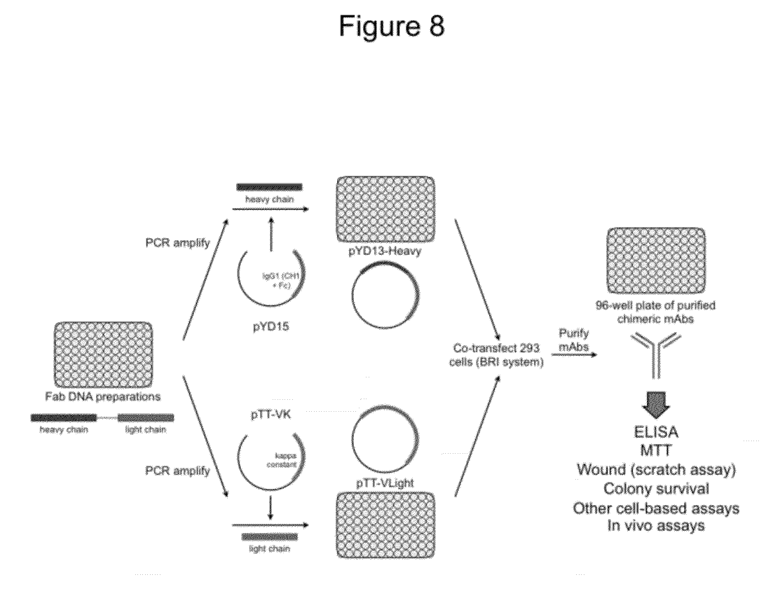

This example discloses the methods used to convert the Fabs into full IgG1 chimeric monoclonal antibodies. A scheme of the methodology is presented in FIG. 8. While three representative monoclonal antibodies were selected for experimentation purposes, the Applicant has generated several others anti-KAAG1 antibodies.

The nucleotide sequence of the complete light chain for the 3D3, 3G10 and 3C4 monoclonal antibodies is outlined in SEQ ID NOs.:3, 7 and 11 respectively, while the amino acid sequence of the complete light chain for the 3D3, 3G10 and 3C4 antibodies is outlined in SEQ ID NOs.: 4, 8 and 12 respectively (CDRS are shown in bold). The nucleotide sequence of the light chain variable region for the 3D3, 3G10 and 3C4 mAbs is outlined in SEQ ID NOs.: 15, 19 and 23 respectively, while the amino acid sequence of the light chain variable region for the 3D3, 3C4 and 3G10 mAbs is outlined in SEQ ID NOs.:17, 21 and 25 respectively.

The nucleotide sequence of the complete heavy chain for the 3D3, 3G10 and 3C4 monoclonal antibodies is outlined in SEQ ID NOs.: 5, 9 and 13 respectively, while the amino acid sequence of the complete heavy chain for the 3D3, 3G10 and 3C4 antibodies is outlined in SEQ ID NOs.: 6, 10 and 14 respectively (CDRS are shown in bold). The nucleotide sequence of the heavy chain variable region for the 3D3, 3G10 and 3C4 mAbs is outlined in SEQ ID NOs.: 16, 20 and 24 respectively, while the amino acid sequence of the heavy chain variable region for the 3D3, 3C4 and 3G10 mAbs is outlined in SEQ ID NOs.:18, 22 and 26 respectively.

Aside from the possibility of conducting interaction studies between the Fab monoclonals and the KAAG1 protein, the use of Fabs is limited with respect to conducting meaningful in vitro and in vivo studies to validate the biological function of the antigen. Thus, it was necessary to transfer the light and heavy chain variable regions contained in the Fabs to full antibody scaffolds, to generate mouse-human chimeric IgG1s. The expression vectors for both the light and heavy immunoglobulin chains were constructed such that i) the original bacterial signal peptide sequences upstream of the Fab expression vectors were replaced by mammalian signal peptides and ii) the light and heavy chain constant regions in the mouse antibodies were replaced with human constant regions. The methods to accomplish this transfer utilized standard molecular biology techniques that are familiar to those skilled in the art. A brief overview of the methodology is described here (see FIG. 8).

Light chain expression vector—an existing mammalian expression plasmid, called pTTVH8G (Durocher et al., 2002), designed to be used in the 293E transient transfection system was modified to accommodate the mouse light chain variable region. The resulting mouse-human chimeric light chain contained a mouse variable region followed by the human kappa constant domain. The cDNA sequence encoding the human kappa constant domain was amplified by PCR with primers OGS1773 and OGS1774 (SEQ ID NOS:52 and 53, respectively). The nucleotide sequence and the corresponding amino acid sequence for the human kappa constant region are shown in SEQ ID NOS:54 and 55, respectively. The resulting 321 base pair PCR product was ligated into pTTVH8G immediately downstream of the signal peptide sequence of human VEGF A (NM_{—}003376). This cloning step also positioned unique restriction endonuclease sites that permitted the precise positioning of the cDNAs encoding the mouse light chain variable regions. The sequence of the final expression plasmid, called pTTVK1, is shown in SEQ ID NO.:56. PCR primers specific for the light chain variable regions of antibodies 3D3, 3G10, and 3C4 (SEQ ID NOS:15, 19, and 23, respectively) were designed that incorporated, at their 5′-end, a sequence identical to the last 20 base pairs of the VEGF A signal peptide. The sequences of these primers are shown in SEQ ID NOS:57, 58, and 59. The same reverse primer was used to amplify all three light chain variable regions since the extreme 3′-ends were identical. This primer (SEQ ID NO.:60) incorporated, at its 3′-end, a sequence identical to the first 20 base pairs of the human kappa constant domain. Both the PCR fragments and the digested pTTVK1 were treated with the 3′-5′ exonuclease activity of T4 DNA polymerase resulting in complimentary ends that were joined by annealing. The annealing reactions were transformed into competent E. coli and the expression plasmids were verified by sequencing to ensure that the mouse light chain variable regions were properly inserted into the pTTVK1 expression vector. Those skilled in the art will readily recognize that the method used for construction of the light chain expression plasmids applies to all anti-KAAG1 antibodies contained in the original Fab library.

Heavy chain expression vector—the expression vector that produced the heavy chain immunoglobulins was designed in a similar manner to the pTTVK1 described above for production of the light chain immunoglobulins. Plasmid pYD11 (Durocher et al., 2002), which contains the human IgGK signal peptide sequence as well as the CH2 and CH3 regions of the human Fc domain of IgG1, was modified by ligating the cDNA sequence encoding the human constant CH1 region. PCR primers OGS1769 and OGS1770 (SEQ ID NOS:61 and 62), designed to contain unique restriction endonuclease sites, were used to amplify the human IgG1 CH1 region containing the nucleotide sequence and corresponding amino acid sequence shown in SEQ ID NOS:63 and 64. Following ligation of the 309 base pair fragment of human CH1 immediately downstream of the IgGK signal peptide sequence, the modified plasmid (SEQ ID NO.:65) was designated pYD15. When a selected heavy chain variable region is ligated into this vector, the resulting plasmid encodes a full IgG1 heavy chain immunoglobulin with human constant regions. PCR primers specific for the heavy chain variable regions of antibodies 3D3, 3G10, and 3C4 (SEQ ID NOS:17, 21, and 25, respectively) were designed that incorporated, at their 5′-end, a sequence identical to the last 20 base pairs of the IgGK signal peptide. The sequences of these primers are shown in SEQ ID NOS:66 (3D3 and 3G10 have the same 5′-end sequence) and 67. The same reverse primer was used to amplify all three heavy chain variable regions since the extreme 3′-ends were identical. This primer (SEQ ID NO.:68) incorporated, at its 3′-end, a sequence identical to the first 20 base pairs of the human CH1 constant domain. Both the PCR fragments and the digested pYD15 were treated with the 3′-5′ exonuclease activity of T4 DNA polymerase resulting in complimentary ends that were joined by annealing. The annealing reactions were transformed into competent E. coli and the expression plasmids were verified by sequencing to ensure that the mouse heavy chain variable regions were properly inserted into the pYD15 expression vector. Those skilled in the art will readily recognize that the method used for construction of the heavy chain expression plasmids applies to all anti-KAAG1 antibodies contained in the original Fab library.

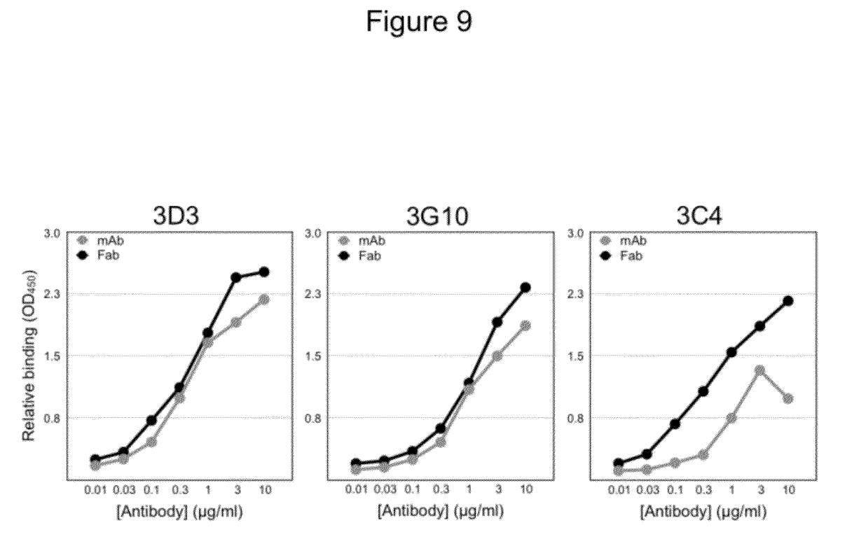

Expression of human IgG1s in 293E cells—The expression vectors prepared above that encoded the light and heavy chain immunoglobulins were expressed in 293E cells using the transient transfection system (Durocher et al., 2002). The methods used for co-transfecting the light and heavy chain expression vectors were described in Example 5. The ratio of light to heavy chain was optimized in order to achieve the most yield of antibody in the tissue culture medium and it was found to be 9:1 (L:H). The ability of the chimeric anti-KAAG1 monoclonal antibodies to bind to recombinant Fc-KAAG1 was measured in the ELISA and compared with the original mouse Fabs. The method was described in Example 5. As depicted in FIG. 9, the binding of the 3D3, and 3G10 chimeric IgG1 monoclonal antibodies was very similar to the Fabs. In the case of the 3C4, the binding activity of the chimeric was slightly less than the Fab. Despite this, this result shows that the transposition of the variable domains from the mouse Fabs into a human IgG1 backbone did not significantly affect the capacity of the light and heavy chain variable regions to confer KAAG1 binding.

### Example 8

This example describes the use of anti-KAAG1 antibodies to block the activity of KAAG1 in ovarian cancer cell models.

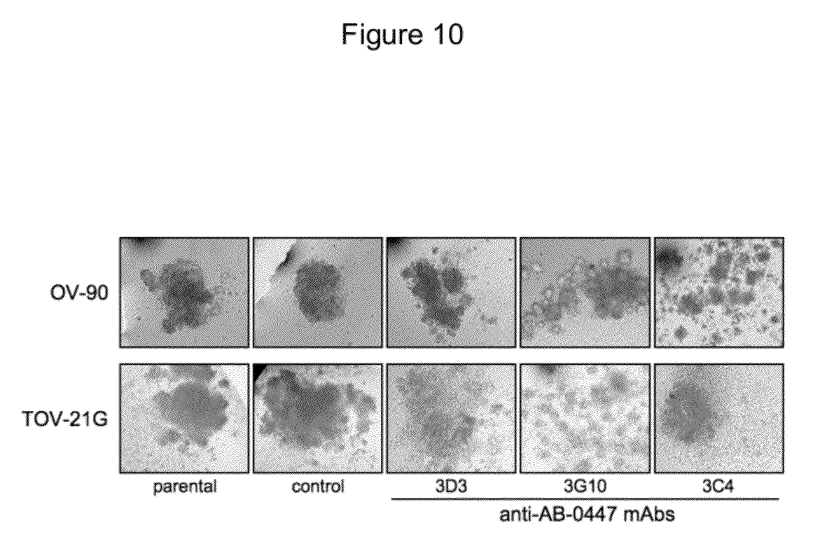

Example 3 disclosed RNAi studies showing that KAAG1 played an important role in the behavior of ovarian cancer cells. The monoclonal antibodies described above were used to determine whether it was possible to reproduce these results by targeting KAAG1 at the cell surface. TOV-21G and OV-90 cells were cultured under conditions to produce spheroids and treated with 10 μg/ml of 3D3, 3G10, or 3C4 anti-KAAG1 chimeric monoclonal antibody. As illustrated in FIG. 10, both cell lines efficiently formed spheroids when left untreated (parental) or when treated with antibody dilution buffer (control). In contrast, the presence of anti-KAAG1 antibodies resulted in loosely packed structures and in certain cases, the cells were unable to assemble into spheroids. These results confirm the earlier observations and suggest that the anti-KAAG1 monoclonal antibodies can modulate the activity of KAAG1 during the formation of spheroids. Since spheroid formation by cancer cell lines is an in vitro model for tumor formation, the results also suggest that blocking KAAG1 could lead to decreased tumor formation in vivo.

### Example 9

This example describes the use of anti-KAAG1 antibodies for detecting the expression of KAAG1 in ovarian tumors.

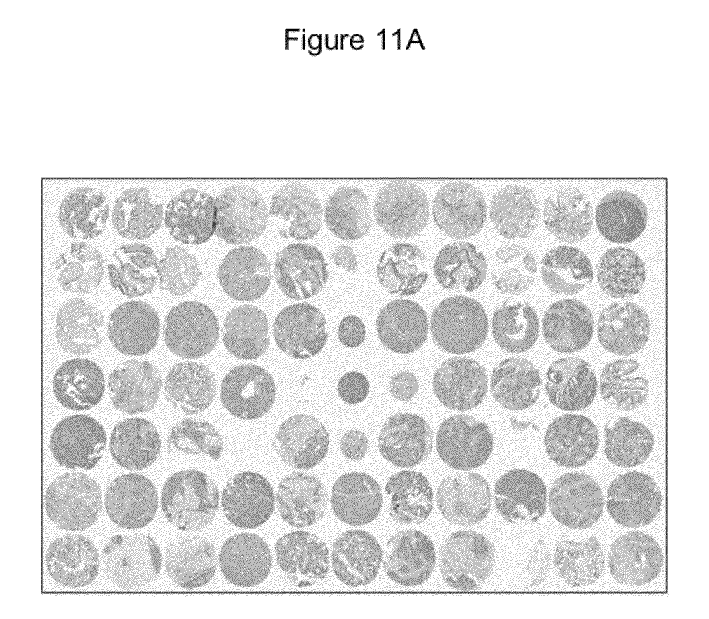

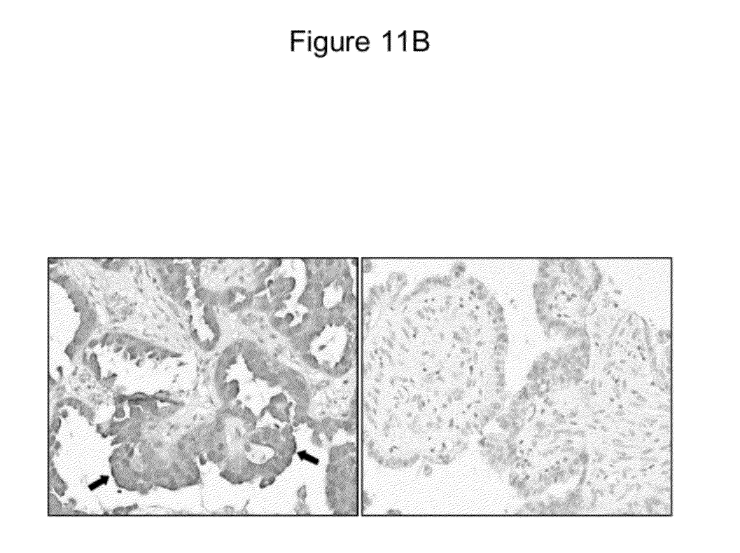

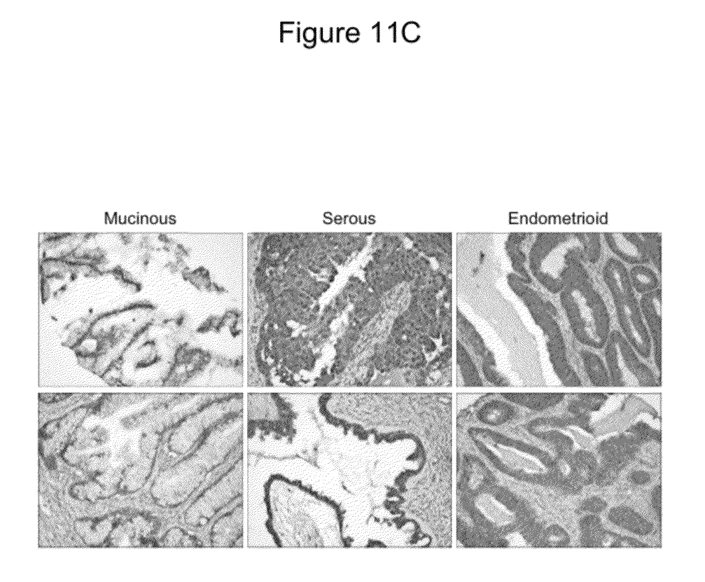

As a means of confirming the expression of KAAG1 protein in ovarian cancer tumors and in order determine if expression of the gene correlated with the presence of the protein, immunohistochemistry was conducted. Tissue microarrays were obtained that contained dozens of ovarian tumor samples generated from patient biopsies. Paraffin-embedded epithelial ovarian tumor samples were placed on glass slides and fixed for 15 min at 50° C. Deparaffinization was conducted by treating 2× with xylene followed by dehydration in successive 5 min washes in 100%, 80%, and 70% ethanol. The slides were washed 2× in PBS for 5 min and treated with antigen retrieval solution (citrate-EDTA) to unmask the antigen. Endogenous peroxide reactive species were removed by incubating slides with H₂O₂ in methanol and blocking was performed by incubating the slides with serum-free blocking solution (Dakocytomation) for 20 min at room temperature. The primary mAb (anti-KAAG1 3D3) was added for 1 h at room temperature. KAAG1-reactive antigen was detected by incubating with biotin-conjugated mouse anti-kappa followed by streptavidin-HRP tertiary antibody. Positive staining was revealed by treating the slides with DAB-hydrogen peroxide substrate for less than 5 min and subsequently counterstained with hematoxylin. The KAAG1 protein was found to be expressed at very high levels in the vast majority of ovarian tumor samples. A representative array containing 70 tumors is depicted in FIG. 11A. As demonstrated by the expression profiling studies that were performed using RT-PCR, KAAG1 transcripts were present in greater than 85% of ovarian tumor samples analyzed. Clearly, there is an excellent correlation between the transcription of the KAAG1 gene and the presence of the protein in ovarian cancer. Some of the samples were inspected at a higher magnification to determine which cells were expressing the KAAG1 protein. As depicted in FIG. 11B, KAAG1 is predominantly expressed in the surface epithelium of ovarian tumors. In addition, strong intensity was observed on the apical side of these epithelial cells (see arrows in FIG. 11B, magnification: 20×). Finally, immunohistochemistry was repeated on ovarian tumor samples that originated from different histotypes. As explained earlier, epithelial ovarian cancer can be classified into 4 major histotypes: serous, endometroid, clear cell, and mucinous. The expression of KAAG1 was detected in all types of epithelial ovarian cancer, in particular serous and endometroid histotypes (see FIG. 11C).

Taken together, these immunohistochemical studies illustrate the utility of detecting KAAG1 in ovarian cancer with the monoclonal antibodies.

### Example 10

### IgG1 Antibodies Against KAAG1 can Mediate ADCC

Antibody-Dependent Cell Cytotoxicity (ADCC) is a mechanism of cell-mediated immunity whereby effector cells, typically natural killer (NK) cells, of the immune system actively lyse target cells that have been bound by specific antibodies. The interaction between the NK cells and the antibody occurs via the constant Fc domain of the antibody and high-affinity Fcγ receptors on the surface of the NK cells. IgG₁s have the highest affinity for the Fc receptors while IgG₂ mAbs exhibit very poor affinity. For this reason the chimeric antibodies targeting KAAG1 were designed as IgG₁s. This type of effector function that is mediated in this manner can often lead to the selective killing of cancer cells that express high level of antigen on their cell surfaces.

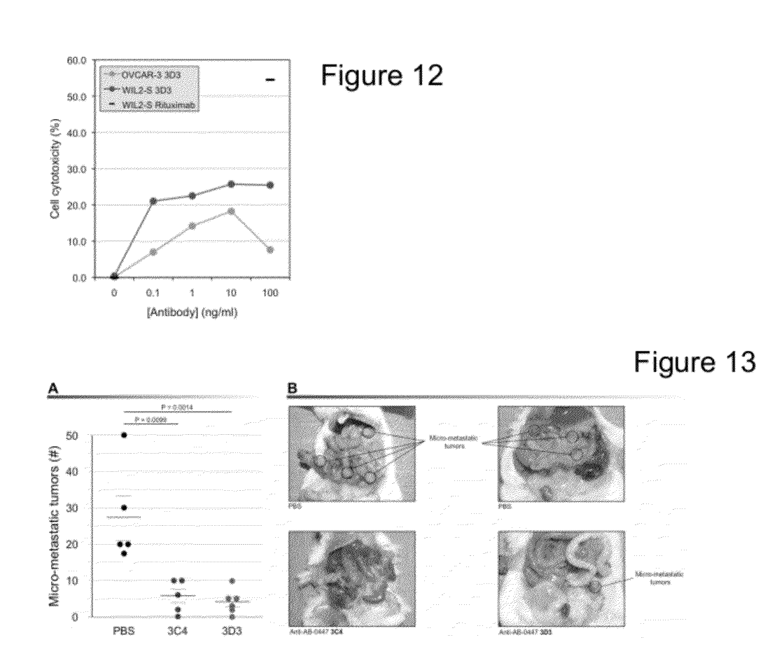

An in vitro assay to measure ADCC activity of the anti-KAAG1 IgG1 chimeric antibodies was adapted from a previously published method, which measured the ADCC activity of the anti-CD20 rituxan in the presence of a lymphoma cell line called WIL2-S (Idusogie et al., (2000) J. Immunol. 164, 4178-4184). Human peripheral blood mononuclear cells (PBMNCs) were used as a source of NK cells which were activated in the presence of increasing concentration of the 3D3 chimeric IgG₁ antibody (see FIG. 12). The target cells were incubated with the activated PBMNCs at a ratio of 1 to 25. As shown, cell death increased in a dose-dependent manner both in the presence of OVCAR-3 and the lymphoma cell line, the latter of which was shown to express KAAG1 by RT-PCR (not shown). As a positive control, the results from the published method were reproduced where high level of ADCC was obtained for rituxan in the presence of WIL2-S cells.

ADCC was also observed with other ovarian cancer cell lines that express relatively high levels of KAAG1. These results demonstrate that IgG1 antibodies that are specific for KAAG1, as exemplified by 3D3, can enhance the lysis of cancer cells which express the antigen on their cell surface.

### Example 11

### Antibodies Against KAAG1 can Reduce the Invasion of Ovarian Tumors

Patients that develop ovarian cancer have lesions that typically initiate by an uncontrolled growth of the cells in the epithelial layer of the ovary or, in some instances, the fallopian tube. If detected early, these primary tumors are surgically removed and first-line chemotherapy can result in very good response rates and improved overall survival. Unfortunately, 70% of the patients will suffer recurrent disease resulting in the spread of hundreds of micro-metastatic tumors throughout the abdominal cavity. Second-line therapies can be efficacious, but often patients either respond poorly or the tumors develop chemoresistance. Treatment options are limited and there are urgent needs for new therapies to circumvent resistance to cytotoxic drugs.

In order to test the efficacy of anti-KAAG1 antibodies in vivo, an animal model of ovarian cancer was used that is the closest representation of the clinical manifestation of the disease in humans. The TOV-112D cell line is of endometrioid origin and expresses the KAAG1 antigen as measured by RT-PCR. Previous IHC studies showed that ovarian tumors of the endometrioid histotype contain strong expression of KAAG1 thus rendering the 112D cell line an appropriate selection for testing anti-KAAG1 antibodies.

The intra-peritoneal inoculation of the TOV-112D cell line in SCID mice resulted in the implantation of dozens of micro-metastatic tumors that closely resemble those that are observed in humans. Mice treated with PBS, the diluent for the antibodies, contained upon examination, an average of 25-30 tumors per animal (FIGS. 13A and B). In some cases, the number of tumors was so high in the abdominal cavity of these mice that the number of tumors could not be easily determine; these mice were excluded from the statistical analysis. When the mice were treated with the 3C4 and 3D3 antibodies, the number of micro-metastatic tumors was drastically reduced. In addition, there was at least one animal per group treated with anti-KAAG1 where no tumors were seen. A second experiment was conducted in mice containing a larger number of TOV-112D tumors (>50/animal) and very similar results were obtained. Moreover, there was very little difference between the groups treated with the 3C4 compared to the 3D3 antibody. However, the tendency in these in vivo experiments as well as the results obtained in the cell-based assays show that the 3D3 antibody displayed slightly more efficacy. Whether, this is due to a more accessible epitope or a higher affinity of 3D3 compared to 3C4 for the antigen still remains to be established. The results from these two experiments demonstrated that targeting KAAG1 on the surface of ovarian cancer cells could lead to a significant reduction in the spread of the tumors in vivo.

Furthermore, these findings are in complete agreement with the observations that were made in the cell-based assays. For example, the increased expression of the KAAG1 mRNA in the spheroids compared to cell lines grown as monolayers; the reduction in cell migration in the presence of KAAG1 shRNAs, the reduction in the ability of cell lines to form spheroids when treated with KAAG1 antibodies; and finally, enhancement of ADCC activity by anti-KAAG1 IgG₁s. Taken together, the results strongly suggest that targeting KAAG1 with an antibody has great therapeutic potential in recurrent ovarian cancer.

### Example 12

### KAAG1 is Expressed in Skin Tumors and Renal Cell Carcinomas and is a Therapeutic Target in these Indications

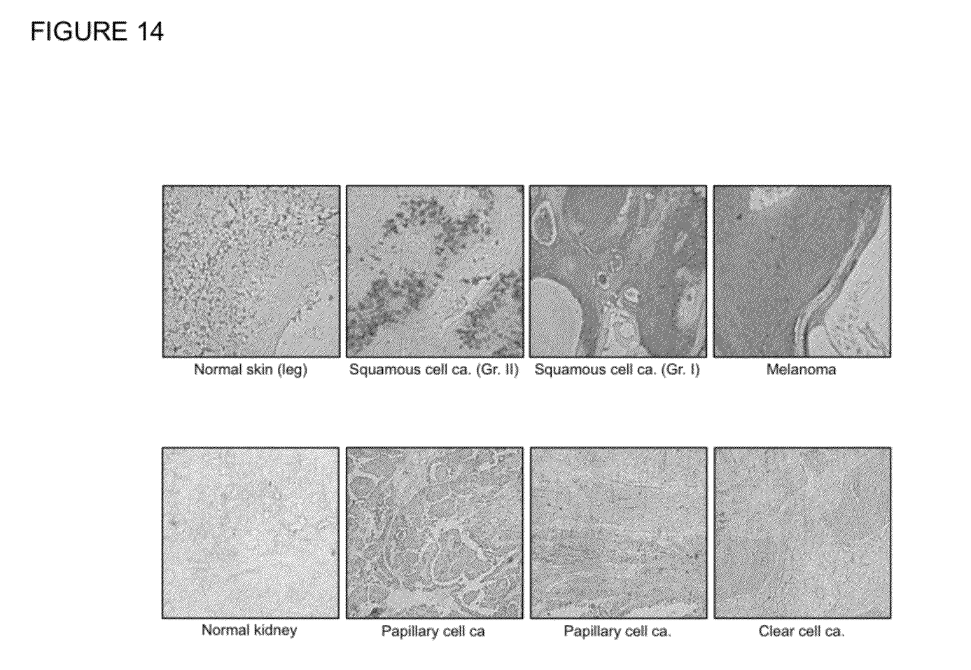

The mRNA profiling studies that were conducted showed that the transcript encoding the KAAG1 antigen was highly expressed in cell lines derived from melanoma samples and renal carcinomas. These results were disclosed in Sooknanan et al., 2007. To confirm the transcriptional regulation of the KAAG1 gene in these cancer types, immunohistochemistry was performed with an anti-KAAG1 antibody on human skin tumor tissue microarrays (Pantomics Inc., Richmond, Calif.) containing several sections isolated from squamous cell carcinomas and melanomas. The analysis of this array showed that there was very strong staining in biopsies isolated from squamous cell carcinomas and melanomas (FIG. 14, top panel). Both of these types are among the most common forms of skin cancers and interestingly, the squamous cell carcinomas are the most metastatic, a fact that again links the expression of KAAG1 to an invasive phenotype. As previously observed, the presence of KAAG1 was very weak or absent on the three normal skin samples that were contained on the array. Similarly, KAAG1 was detected in many of the samples contained in an array of renal cancer. Most of the positive samples were predominantly of the papillary cell carcinoma type and a few clear cell carcinomas expressed KAAG1 protein. Papillary carcinomas represent approximately 20% of renal cancer cases.

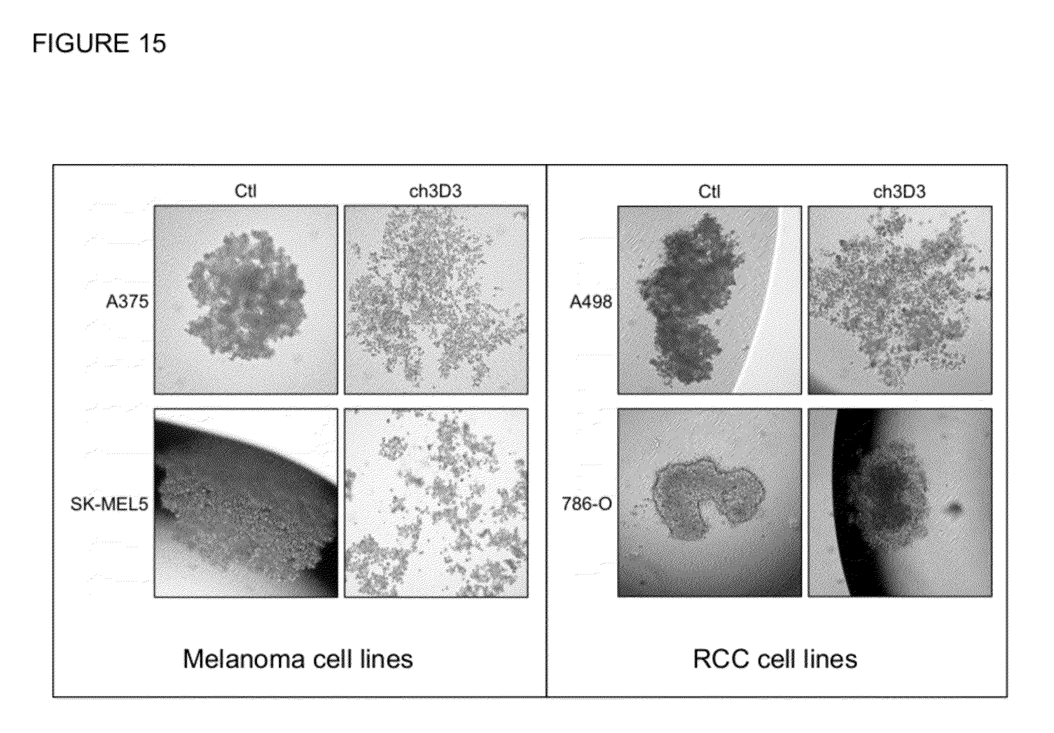

In order to test if the function of KAAG1 is the same in these types of cancer compared to its role in ovarian cancer, cell lines derived from melanoma and renal cell carcinomas were obtained and tested in the spheroid culture assay (see Example and 8). For the melanoma model, A375 and SK-MEL5 cells, two malignant melanoma cell lines, were cultured under conditions that allowed them to form spheroids in the presence of 5% FBS. The cultures were incubated with or without the anti-KAAG1 chimeric 3D3 antibody at a concentration of 5 μg/ml. As shown in FIG. 15, inclusion of 3D3 antibody in the cultures prevented the proper assembly of spheroid structures in melanoma cell lines. This result suggested that KAAG1 plays a similar role in melanoma as it does in ovarian cancer. Cell lines derived from renal cell carcinoma were also tested. The A-498 cell line is a renal papillary cell carcinoma cell line whereas the 786-0 is a renal clear cell carcinoma. As depicted in FIG. 15, only the A-498 spheroids were affected by the presence of the 3D3 anti-KAAG1 antibody while the 786-0 cell line was unaffected in this assay. These results parallel the immunohistochemistry results described above and indicate that the inhibition of spheroids formation is dependent on the presence of KAAG1 on the surface of renal cancer cells derived predominantly from papillary kidney cancers. It is possible however, that the anti-KAAG1 antibody may work in other types of assays for renal clear cell carcinoma.

Taken together, these data are strongly supportive of a critical function in role of KAAG1 in melanoma and kidney cancer and indicate that blocking KAAG1 with antibodies in these indications has therapeutic potential.

### Example 13

### KAAG1 is Expressed on the Surface of Ovarian Cancer Cells

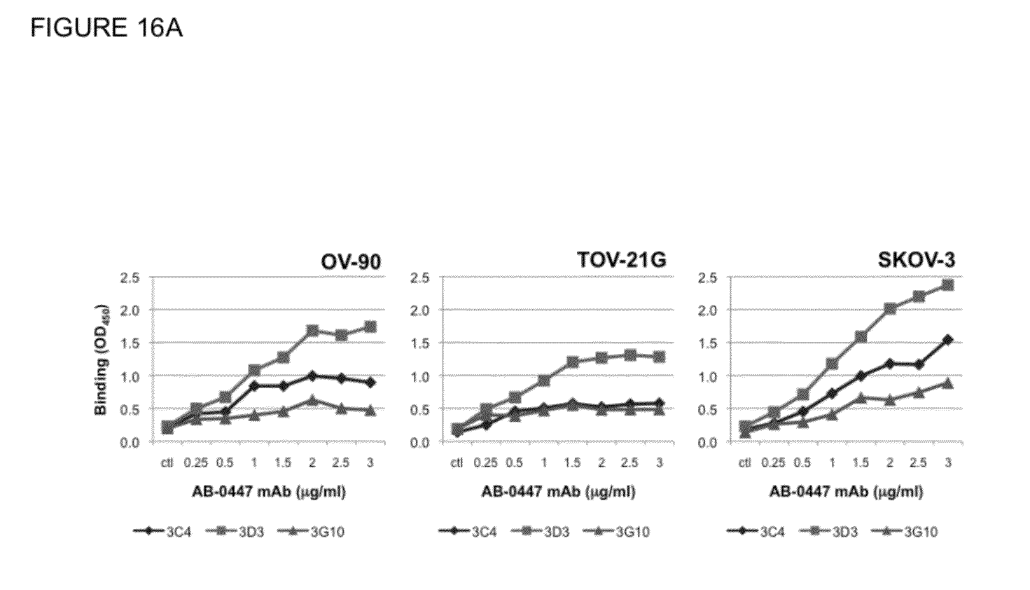

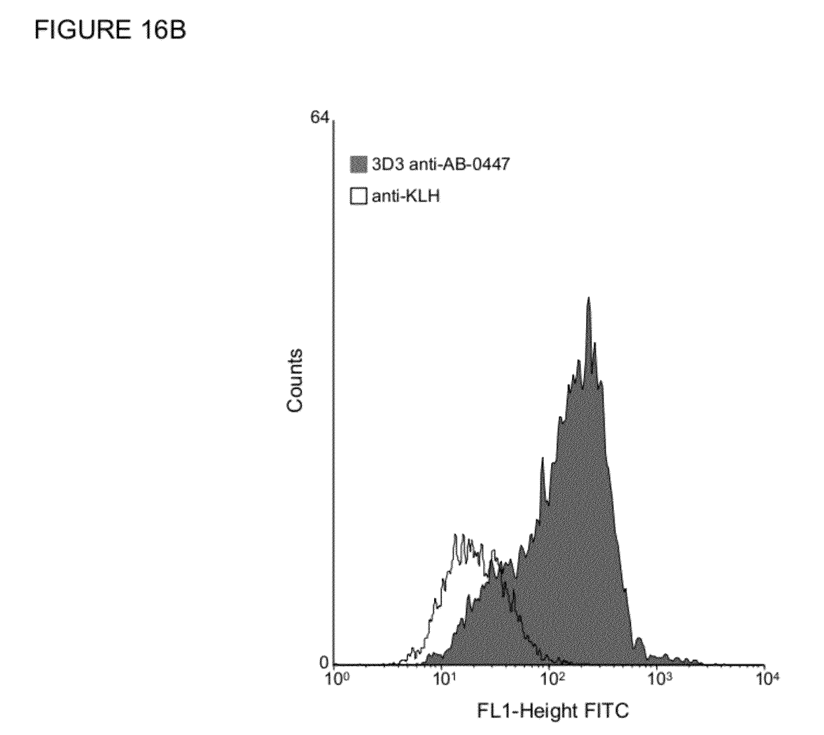

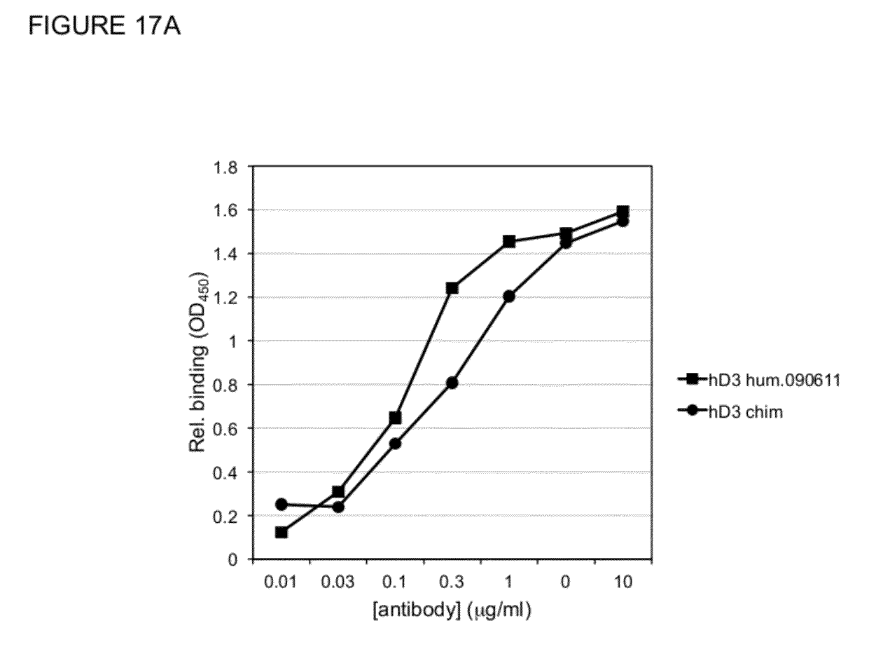

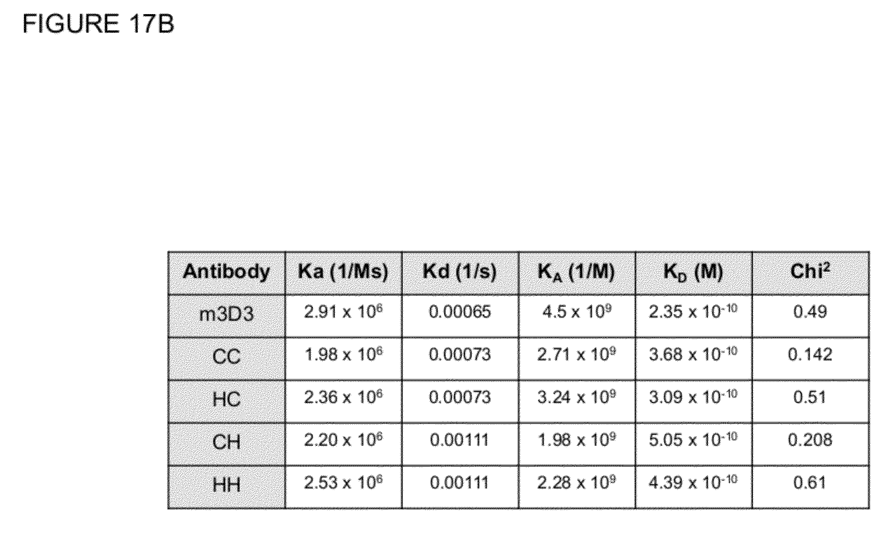

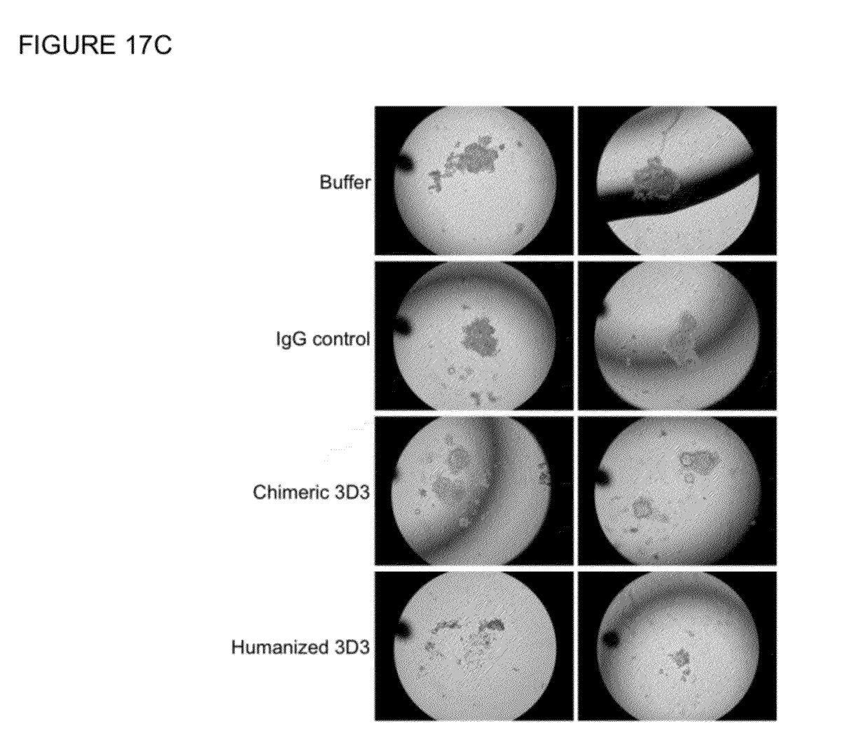

The combined results from the bioinformatics analysis of the primary structure of the cDNA encoding KAAG1, biochemical studies, and immunohistochemical detection of the protein in epithelial cells suggested that the KAAG1 antigen was located on the cell surface. However, more direct evidence was required to demonstrate that KAAG1 is indeed a membrane-bound protein. In one approach, ovarian cancer cell lines known to express KAAG1 were plated in micro-titer plates, fixed under conditions that do not permeate the cells, and incubated with increasing concentration of anti-KAAG1 chimeric antibodies. Following extensive washing of the cells, bound antibody was detected with HRP-conjugated anti-human IgG as a secondary antibody in a modified cell-based ELISA (see FIG. 16A). The first observation that can be made from these experiments is that the antibodies could be specifically captured by the cells suggesting that the KAAG1 was present at the cell surface. Secondly, the amount of binding was strongest on SKOV-3 cells and the TOV-21G cells exhibited the weakest binding. This was in complete agreement with RT-PCR data which demonstrated that the KAAG1 mRNA was expressed in similar proportions in these cell lines (not shown). Additionally, the 3D3 antibody produced the strongest signal implying that the epitope targeted by this antibody was the most accessible in this assay. The 3G10 could only detect KAAG1 in the cell line that expressed the highest level of AB-0447 (SKOV-3 cells, see right panel of FIG. 16A). A second approach used was flow cytometry. In this case, a mouse 3D3 anti-KAAG1 antibody was incubated with SKOV-3 ovarian cancer cells at saturating conditions and following extensive washing, the bound 3D3 anti-KAAG1 antibody was detected with anti-mouse IgG conjugated to FITC in a flow cytometer. As shown in FIG. 16B, the signal at the surface of SKOV-3 cells was much higher compared to same cells labeled with the negative control, an anti-KLH (Keyhole limpet hemocyanin) antibody, specific for a non-mammalian unrelated protein, which was at a fluorescence level the same as the background readings. Taken together, these results demonstrate that KAAG1 is located on the surface of cells.

### Example 14

### Methods for the Use of Humanized Anti-KAAG1 Antibodies

On the basis of both the in vitro and preliminary in vivo results, two mouse anti-KAAG1 antibody candidates, designated 3D3 and 3C4, were selected for humanization using in silico modeling using methods familiar to those in the art. In brief, the variable regions of the murine antibodies were modeled in 3D based on available crystal structures of mouse, humanized, and fully human variable regions that displayed high sequence homology and similar CDR loop lengths. The CDRs are the amino acid sequences that contribute to antigen binding; there are 3 CDRs on each antibody chain. Additionally, the framework regions, the amino acid sequences that intervene between the CDRs, were modified by standard homology comparison between mouse and human antibody sequences resulting in the ‘best-fit’ human sequence. These modifications ensured that the proper positioning of the CDR loops was maintained to ensure maximum antigen binding in the humanized structure as well as preserving the potential N- and O-linked glycosylation sites. The sequence of both the heavy and light chain variable regions in the humanized (h) 3D3 and 3G4 resulted in 96% and 94% humanization, respectively. The 3D3 required the maintenance of 3 unusual amino acids (Met93 and Gly94 on the heavy chain and Ser57 on the light chain) because of their proximity to the CDRs. Modeling predicted that replacement of these mouse amino acids with human equivalents might compromise binding of the antibody with the KAAG1 antigen. In the case of 3C4, 6 amino acids were considered unusual (Glut Gln72 and Ser98 on the heavy chain and Thr46, Phe49 and Ser87 on the light chain). In both figures, the light chain CDRs are indicated by L1, L2, and L3 for CDR1, CDR2, and CDR3, respectively, whereas the heavy chain CDRs are indicated by H1, H2, and H3 for CDR1, CDR2, and CDR3, respectively.

The sequences that encode the complete anti-KAAG1 3D3 immunoglobulin light and heavy chains are shown in SEQ ID NO.:69 and 70, respectively. The variable region of the humanized 3D3 light chain is contained between amino acids 21-133 of SEQ ID NO.:69 and is shown in SEQ ID NO.:71. The variable region of the humanized 3D3 heavy chain is contained between amino acids 20-132 of SEQ ID NO.:70 and is shown in SEQ ID NO.:72. The sequences that encode the complete anti-KAAG1 3C4 immunoglobulin light and heavy chains are shown in SEQ ID NO.:73 and 74, respectively. The variable region of the humanized 3C4 light chain is contained between amino acids 21-127 of SEQ ID NO.:73 and is shown in SEQ ID NO.:75. The variable region of the humanized 3C4 heavy chain is contained between amino acids 19-136 of SEQ ID NO.:74 and is shown in SEQ ID NO.:76.

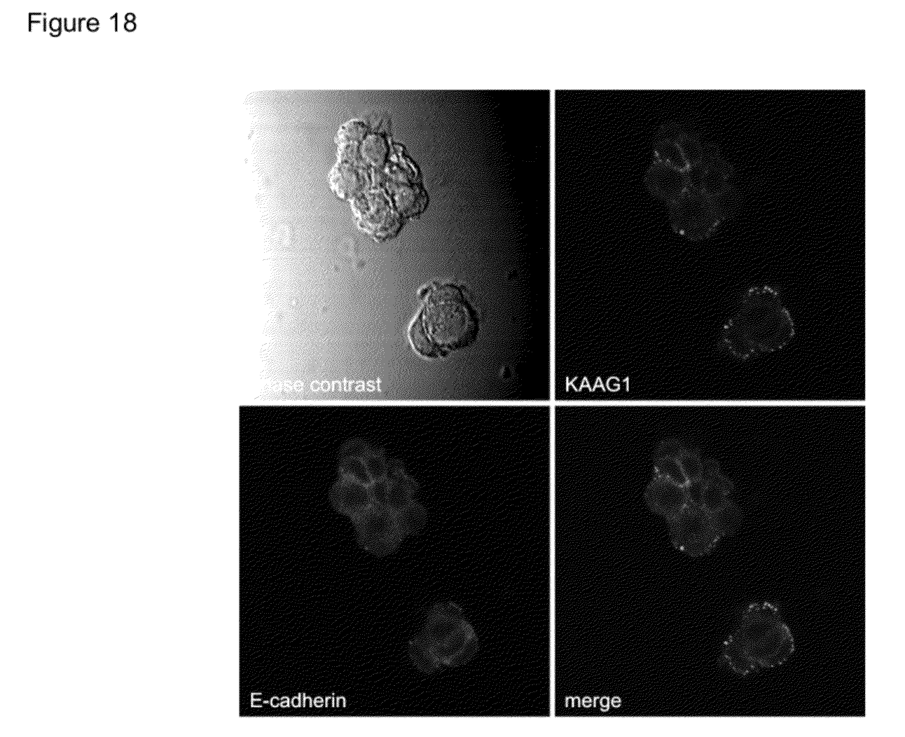

Following assembly of expression vectors and production of the h3D3 in transfected mammalian cells (see Example 7), several assays were performed to demonstrate the bio-equivalence of the humanization process. Since an antibody harboring effector functions was required, the h3D3 was assembled as a human IgG₁. ELISA-based assays were performed to directly compare the ability of the h3D3 to recombinant KAAG1. The methods used to perform these tests were as described in Example 5 using recombinant Fc-KAAG1. As shown in FIG. 18A, the binding activity of the h3D3 was identical to that of the chimeric 3D3.

More precise measurements were conducted using Surface Plasmon Resonance (SPR) in a Biacore instrument. Kinetic analysis was used to compare the affinity of the chimeric 3D3 with the h3D3 as well as with hybrid antibodies encompassing different permutations of the light and heavy chains (see FIG. 18B). Briefly, anti-human Fc was immobilized on the Biacore sensor chip and chimeric or h3D3 was captured on the chip. Different concentrations of monomeric recombinant KAAG1 were injected and the data were globally fitted to a simple 1:1 model to determine the kinetic parameters of the interaction. The kinetic parameters of the chimeric 3D3 were tabulated in FIG. 18B (m3D3). The average K_{D} of the chimeric 3D3 was 2.35×10⁻¹⁰ M. In comparison, all permutations of the chimeric(C)/humanized(H) displayed very similar kinetic parameters. The average K_{D} of the chimeric light chain expressed with the chimeric heavy chain (indicated as ‘CC’ in FIG. 18B) was 2.71×10⁻¹⁰ M, the average K_{D} of the humanized light chain expressed with the chimeric heavy chain (indicated as ‘HC’ in FIG. 18B) was 3.09×10⁻¹⁰ M, the average K_{D} of the chimeric light chain expressed with the humanized heavy chain (indicated as ‘CH’ in FIG. 18B) was 5.05×10⁻¹⁰ M, and the average K_{D} of the humanized light chain expressed with the humanized heavy chain (indicated as ‘HH’ in FIG. 18B) was 4.39×10⁻¹⁰ M. The analyses indicated that the humanization of 3D3 conserved the binding activity of the original mouse antibody.

The biological function of the h3D3 was evaluated in the spheroid culture assay (see Example 8). SKOV-3 ovarian cancer cells were cultured in the presence of 5% FBS in the presence of h3D3 or a non-KAAG1 binding isotype control antibody. The results (shown in FIG. 18C), indicated that treatment with either the buffer or the non-related IgG did not inhibit the formation of the compact 3-D structures. In contrast, both the chimeric 3D3 and the humanized 3D3 prevented the spheroids from forming. The results are shown in duplicate (left and right panels). These results indicate that the biological activity of the chimeric 3D3 was conserved in the humanized 3D3 and suggests that the h3D3 will behave in an identical manner.

### Example 15

### Methods for Use of Anti-KAAG1 Antibodies as Antibody Conjugates

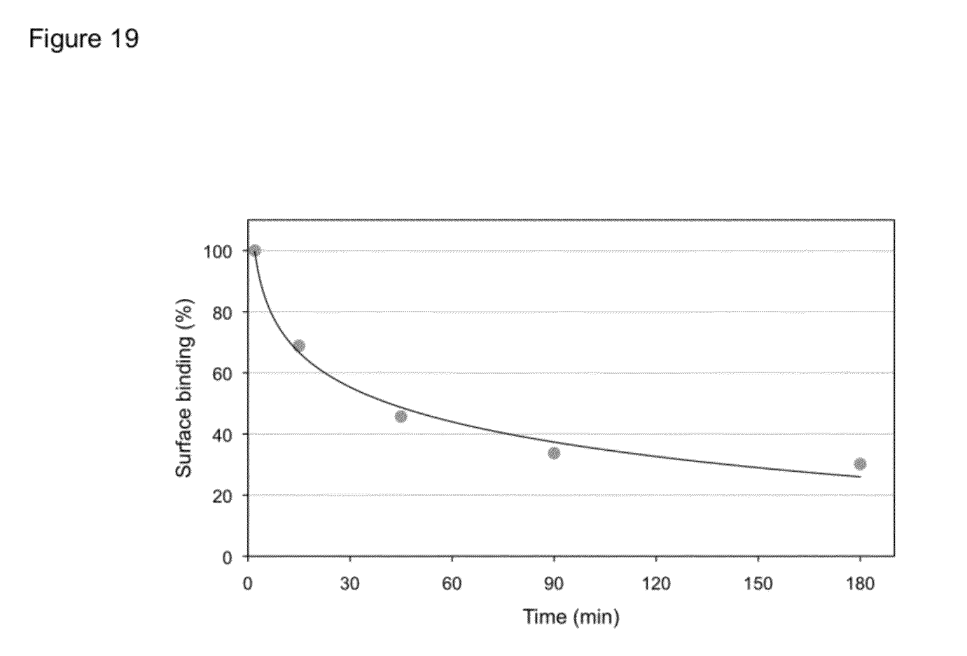

As demonstrated above, the KAAG1 antigen was detected on the surface of ovarian cancer cells using a cell-based ELISA method and flow cytometry. To further substantiate these findings, fluorescence microscopy was used to visualize the antigen-antibody complex on the surface of cells. OVCAR-3 cells were seeded on coverslips and grown O/N. The chimeric 3D3 IgG1 anti-KAAG1 antibody was added to the coverslips and incubated at 37 C, 5% CO₂ for no longer than 2 h. The cells were fixed in para-formaldehyde and stained with human anti-IgG-FITC for 30 min at RT. After washing, the cells were observed using a confocal microscope. The antibody bound to the surface of cells was detected by incubating the coated cells in the presence of FITC-labeled anti-human IgG. As displayed in FIG. 19, the pattern of staining is indicative of an enrichment at the cells surface (see upper right panel, KAAG1). Furthermore, staining with an antibody specific for E-cadherin, a known membrane protein, showed that the staining of E-cadherin co-localized with that of KAAG1 (see lower panels, E-cadherin and merge). Similar results were seen with SKOV-3 and TOV-112D cells, two other cell lines that are positive for KAAG1 expression. These data confirm that KAAG1 is detectable on the surface of ovarian cancer cells and that the 3D3 antibody can be used to bind to the antigen on these cells. Therefore, by several different methods, it was established that the protein encoded by the KAAG1 gene can be detected at the surface of ovarian cancer cells.

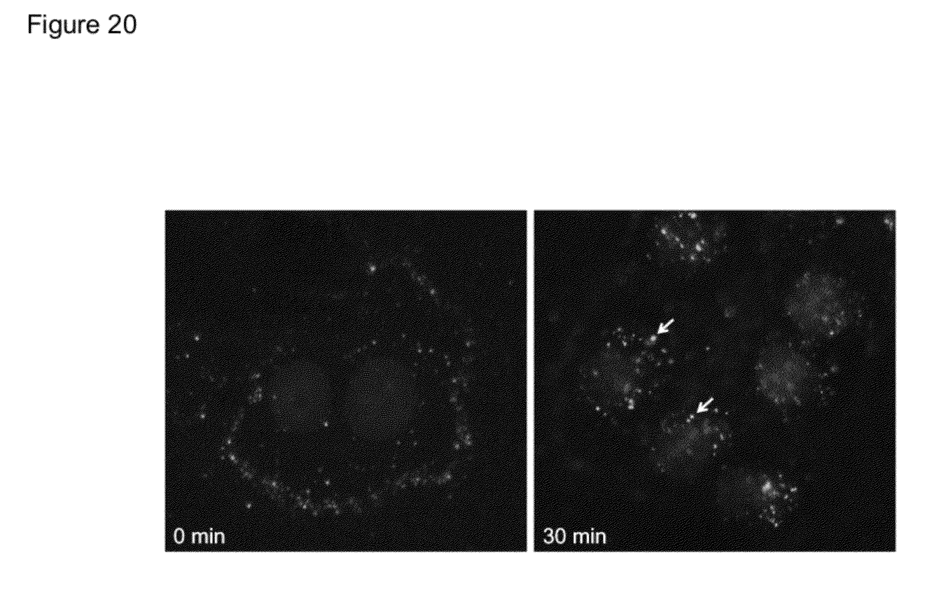

There are several different molecular events that can occur upon binding of an antibody to its target on the surface of cells. These include i) blocking accessibility to another cell-surface antigen/receptor or a ligand, ii) formation of a relatively stable antibody-antigen complex to allow cells to be targeted via ADCC or CDC, iii) signaling events can occur as exemplified by agonistic antibodies, iv) the complex can be internalized, or v) the complex can be shed from the cell surface. To address this question we wished to examine the behavior of the antibody-antigen complex on the surface of the cells as it pertains to KAAG1. SKOV-3 cells were plated, washed, and incubated with 5 μg/ml chimeric 3D3 antibody. After washing, complete OSE medium was added and the cells placed at 37 C for up to 180 minutes. The cells were removed at the indicated times, rapidly cooled, and prepared for cytometry with FITC-conjugated anti-human IgG and the results were expressed as the percentage of mean fluorescence intensity (MFI, % surface binding) remaining. As illustrated in FIG. 20, the fluorescence signal decreases over a period of 30-45 minutes. This result indicates that the complex between the 3D3 antibody and KAAG1 exhibits stability on the surface of SKOV-3 cells. However, it is evident from this experiment that the complex slowly disappeared from the cells which indicated that an internalization of the complex had occurred. Preliminary studies to elucidate the mechanism responsible for this decrease in cell-surface fluorescence have revealed that the complex appears to be internalized.

These findings were further confirmed by conducting immunofluorescence on live cells to see if this internalization could be microscopically observed. OVCAR-3 cells were seeded on cover slips in full medium (OSE medium (Wisent) containing 10% FBS, 2 mM glutamine, 1 mM sodium-pyruvate, 1× non-essential amino acids, and antibiotics. Once the cells were properly adhered, the medium was removed and the cover slips washed twice gently with ice-cold PBS containing 1% FBS, 1 mM MgCl2, 1 mM CaCl2. Chimeric 3D3 (human IgG1) at 10 μg/ml was added to the cells and incubated on ice for 1 h. The coverslips were incubated for 30 minutes full medium at 37 C and following washing in PBS, they were fixed at room temperature in 3.7% formaldehyde (in PBS) containing saponin for 30 min. AB-3D3 was visualized with rabbit anti-human IgG-488 Daylight (Jackson ImmunoResearch) diluted 1:400 and mounted on microscope slides using Gold Antifade reagent with DAPI (Invitrogen). As seen in FIG. 21, the 3D3 antibody is able to detect complexes predominantly on the cell surface at time 0 (FIG. 21, left panel) but after 30 minutes incubation at 37 C, these complexes were detected inside the cells (see FIG. 21, right panel, arrows). This data is in complete agreement with the flow cytometry data (see FIG. 21) and confirms that the KAAG1/3D3 complex is internalized in cells. Finally, in additional studies, it was found that the KAAG1/3D3 complexes co-localize with early endosome antigen 1 (EEA1) in SKOV-3 cells. EEA1 is a protein known to exist in vesicles that occur during endosomal trafficking.

In other experiments, the 3D3 antibody was conjugated with doxorubicin and administered to tumor-bearing mice. The antibody conjugate was shown to be cytotoxic to tumor cells (data not shown).

Taken together, these studies demonstrated that antibodies specific for KAAG1 might have uses as an antibody-drug conjugate (ADC). Thus, the high level of ovarian cancer specificity of KAAG1 coupled with the capacity of this target to be internalized in cells would support the development of applications as an ADC.

One of skill in the art will readily recognize that orthologues for all mammals maybe identified and verified using well-established techniques in the art, and that this disclosure is in no way limited to one mammal. The term “mammal(s)” for purposes of this disclosure refers to any animal classified as a mammal, including humans, domestic and farm animals, and zoo, sports, or pet animals, such as dogs, cats, cattle, horses, sheep, pigs, goats, rabbits, etc. Preferably, the mammal is human.

The sequences in the experiments discussed above are representative of the NSEQ being claimed and in no way limit the scope of the invention. The disclosure of the roles of the NSEQs in proliferation of ovarian cancer cells satisfies a need in the art to better understand ovarian cancer disease, providing new compositions that are useful for the diagnosis, prognosis, treatment, prevention and evaluation of therapies for ovarian cancer and other cancers where said genes are expressed as well.

The art of genetic manipulation, molecular biology and pharmaceutical target development have advanced considerably in the last two decades. It will be readily apparent to those skilled in the art that newly identified functions for genetic sequences and corresponding protein sequences allows those sequences, variants and derivatives to be used directly or indirectly in real world applications for the development of research tools, diagnostic tools, therapies and treatments for disorders or disease states in which the genetic sequences have been implicated.

Although the present invention has been described herein above by way of preferred embodiments thereof, it maybe modified, without departing from the spirit and nature of the subject invention as defined in the appended claims.

Sequences referred in the description

SEQ ID NO.: 1
GAGGGGCATCAATCACACCGAGAAGTCACAGCCCCTCAACCACTGAGGTGTGGGGGGGTAGGGATC
TGCATTTCTTCATATCAACCCCACACTATAGGGCACCTAAATGGGTGGGCGGTGGGGGAGACCGAC
TCACTTGAGTTTCTTGAAGGCTTCCTGGCCTCCAGCCACGTAATTGCCCCCGCTCTGGATCTGGTC
TAGCTTCCGGATTCGGTGGCCAGTCCGCGGGGTGTAGATGTTCCTGACGGCCCCAAAGGGTGCCTG
AACGCCGCCGGTCACCTCCTTCAGGAAGACTTCGAAGCTGGACACCTTCTTCTCATGGATGACGAC
GCGGCGCCCCGCGTAGAAGGGGTCCCCGTTGCGGTACACAAGCACGCTCTTCACGACGGGCTGAGA
CAGGTGGCTGGACCTGGCGCTGCTGCCGCTCATCTTCCCCGCTGGCCGCCGCCTCAGCTCGCTGCT
TCGCGTCGGGAGGCACCTCCGCTGTCCCAGCGGCCTCACCGCACCCAGGGCGCGGGATCGCCTCCT
GAAACGAACGAGAAACTGACGAATCCACAGGTGAAAGAGAAGTAACGGCCGTGCGCCTAGGCGTCC
ACCCAGAGGAGACACTAGGAGCTTGCAGGACTCGGAGTAGACGCTCAAGTTTTTCACCGTGGCGTG
CACAGCCAATCAGGACCCGCAGTGCGCGCACCACACCAGGTTCACCTGCTACGGGCAGAATCAAGG
TGGACAGCTTCTGAGCAGGAGCCGGAAACGCGCGGGGCCTTCAAACAGGCACGCCTAGTGAGGGCA
GGAGAGAGGAGGACGCACACACACACACACACACAAATATGGTGAAACCCAATTTCTTACATCATA
TCTGTGCTACCCTTTCCAAACAGCCTA
SEQ ID NO.: 2
MDDDAAPRVEGVPVAVHKHALHDGLRQVAGPGAAAAHLPRWPPPQLAASRREAPPLSQRPHRTQGA
GSPPETNEKLTNPQVKEK
SEQ ID NO.: 3
GACATTGTGATGACCCAGTCTCCATCCTCCCTGGCTGTGTCAATAGGACAGAAGGTCACTATGAAC
TGCAAGTCCAGTCAGAGCCTTTTAAATAGTAACTTTCAAAAGAACTTTTTGGCCTGGTACCAGCAG
AAACCAGGCCAGTCTCCTAAACTTCTGATATACTTTGCATCCACTCGGGAATCTAGTATCCCTGAT
CGCTTCATAGGCAGTGGATCTGGGACAGATTTCACTCTTACCATCAGCAGTGTGCAGGCTGAAGAC
CTGGCAGATTACTTCTGTCAGCAACATTATAGCACTCCGCTCACGTTCGGTGCTGGGACCAAGCTG
GAGCTGAAAGCTGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCC
TGCGAAGTCACCCATCAGGGCCTGAGCTCGCCCGTCACAAAGAGCTTCAACAGGGGAGAGTGT
SEQ ID NO.: 4
DIVMTQSPSSLAVSIGQKVTMNCKSSQSLLNSNFQKNFLAWYQQKPGQSPKLLIYFASTRESSIPD
RFIGSGSGTDFTLTISSVQAEDLADYFCQQHYSTPLTFGAGTKLELKAVAAPSVFIFPPSDEQLKS
GTASVVCLLNNFYPREAKVQWKVDNALQSGNSQESVTEQDSKDSTYSLSSTLTLSKADYEKHKVYA
CEVTHQGLSSPVTKSFNRGEC
SEQ ID NO.: 5
GAGGTTCAGCTGCAGCAGTCTGTAGCTGAGCTGGTGAGGCCTGGGGCTTCAGTGACGCTGTCCTGC
AAGGCTTCGGGCTACATATTTACTGACTATGAGATACACTGGGTGAAGCAGACTCCTGTGCATGGC
CTGGAATGGATTGGGGTTATTGATCCTGAAACTGGTAATACTGCCTTCAATCAGAAGTTCAAGGGC
AAGGCCACACTGACTGCAGACATATCCTCCAGCACAGCCTACATGGAACTCAGCAGTTTGACATCT
GAGGACTCTGCCGTCTATTACTGTATGGGTTATTCTGATTATTGGGGCCAAGGCACCACTCTCACA
GTCTCCTCAGCCTCAACGAAGGGCCCATCTGTCTTTCCCCTGGCCCCCTCCTCCAAGAGCACCTCT
GGGGGCACAGCGGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTG
AATCACAAGCCCAGCAACACCAAGGTGGACAAGAAAGTTGAGCCCAAATCTTGTGAATTCACTCAC
ACATGCCCACCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCT
CCCGGGAAA
SEQ ID NO.: 6
EVQLQQSVAELVRPGASVTLSCKASGYIFTDYEIHWVKQTPVHGLEWIGVIDPETGNTAFNQKFKG
KATLTADISSSTAYMELSSLTSEDSAVYYCMGYSDYWGQGTTLTVSSASTKGPSVFPLAPSSKSTS
GGTAALGCLVKDYFPEPVTVSWNSGALTSGVHTFPAVLQSSGLYSLSSVVTVPSSSLGTQTYICNV
NHKPSNTKVDKKVEPKSCEFTHTCPPCPAPELLGGPSVFLEPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO.: 7
GATGTTTTGATGACCCAAACTCCACGCTCCCTGTCTGTCAGTCTTGGAGATCAAGCCTCCATCTCT
TGTAGATCGAGTCAGAGCCTTTTACATAGTAATGGAAACACCTATTTAGAATGGTATTTGCAGAAA
CCAGGCCAGCCTCCAAAGGTCCTGATCTACAAAGTTTCCAACCGATTTTCTGGGGTCCCAGACAGG
TTCAGTGGCAGTGGATCAGGGACAGATTTCACACTCAAGATCAGCGGAGTGGAGGCTGAGGATCTG
GGAGTTTATTACTGCTTTCAAGGTTCACATGTTCCTCTCACGTTCGGTGCTGGGACCAAGCTGGAG
CTGAAAGCTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGGAGAGTGT
SEQ ID NO.: 8
DVLMTQTPRSLSVSLGDQASISCRSSQSLLHSNGNTYLEWYLQKPGQPPKVLIYKVSNRFSGVPDR
FSGSGSGTDFTLKISGVEAEDLGVYYCFQGSHVPLTFGAGTKLELKAVAAPSVFIFPPSDEQLKSG
TASVVCLLNNFYPREAKVQWKVDNALQSGNSQESVTEQDSKDSTYSLSSTLTLSKADYEKHKVYAC
EVTHQGLSSPVTKSFNRGEC
SEQ ID NO.: 9
GAGATCCAGCTGCAGCAGTCTGGACCTGAGTTGGTGAAGCCTGGGGCTTCAGTGAAGATATCCTGT
AAGGCTTCTGGATACACCTTCACTGACAACTACATGAACTGGGTGAAGCAGAGCCATGGAAAGAGC
CTTGAGTGGATTGGAGATATTAATCCTTACTATGGTACTACTACCTACAACCAGAAGTTCAAGGGC
AAGGCCACATTGACTGTAGACAAGTCCTCCCGCACAGCCTACATGGAGCTCCGCGGCCTGACATCT
GAGGACTCTGCAGTCTATTACTGTGCAAGAGATGACTGGTTTGATTATTGGGGCCAAGGGACTCTG
GTCACTGTCTCTGCAGCCTCAACGAAGGGCCCATCTGTCTTTCCCCTGGCCCCCTCCTCCAAGAGC
ACCTCTGGGGGCACAGCGGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGC
AACGTGAATCACAAGCCCAGCAACACCAAGGTGGACAAGAAAGTTGAGCCCAAATCTTGTGAATTC
ACTCACACATGCCCACCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCC
CTGTCTCCCGGGAAA
SEQ ID NO.: 10
EIQLQQSGPELVKPGASVKISCKASGYTFTDNYMNWVKQSHGKSLEWIGDINPYYGTTTYNQKFKG
KATLTVDKSSRTAYMELRGLTSEDSAVYYCARDDWFDYWGQGTLVTVSAASTKGPSVFPLAPSSKS
TSGGTAALGCLVKDYFPEPVTVSWNSGALTSGVHTFPAVLQSSGLYSLSSVVTVPSSSLGTQTYIC
NVNHKPSNTKVDKKVEPKSCEFTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO.: 11
GACATCGTTATGTCTCAGTCTCCATCTTCCATGTATGCATCTCTAGGAGAGAGAGTCACTATCACT
TGCAAGGCGAGTCAGGACATTCATAACTTTTTAAACTGGTTCCAGCAGAAACCAGGAAAATCTCCA
AAGACCCTGATCTTTCGTGCAAACAGATTGGTAGATGGGGTCCCATCAAGGTTCAGTGGCAGTGGA
TCTGGGCAAGATTATTCTCTCACCATCAGCAGCCTGGAGTTTGAAGATTTGGGAATTTATTCTTGT
CTACAGTATGATGAGATTCCGCTCACGTTCGGTGCTGGGACCAAGCTGGAGCTGAGAGCTGTGGCT
GCACCATCTGTCTTCATCTTCCCGCCATCTGATGAGCAGTTGAAATCTGGAACTGCCTCTGTTGTG
TGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
SEQ ID NO.: 12
DIVMSQSPSSMYASLGERVTITCKASQDIHNFLNWFQQKPGKSPKTLIFRANRLVDGVPSRFSGSG
SGQDYSLTISSLEFEDLGIYSCLQYDEIPLTFGAGTKLELRAVAAPSVFIFPPSDEQLKSGTASVV
CLLNNFYPREAKVQWKVDNALQSGNSQESVTEQDSKDSTYSLSSTLTLSKADYEKHKVYACEVTHQ
GLSSPVTKSFNRGEC
SEQ ID NO.: 13
GAGGTGCAGCTTCAGGAGTCAGGACCTGACCTGGTGAAACCTTCTCAGTCACTTTCACTCACCTGC
ACTGTCACTGGCTTCTCCATCACCAGTGGTTATGGCTGGCACTGGATCCGGCAGTTTCCAGGAAAC
AAACTGGAGTGGATGGGCTACATAAACTACGATGGTCACAATGACTACAACCCATCTCTCAAAAGT
CGAATCTCTATCACTCAAGACACATCCAAGAACCAGTTCTTCCTGCAGTTGAATTCTGTGACTACT
GAGGACACAGCCACATATTACTGTGCAAGCAGTTACGACGGCTTATTTGCTTACTGGGGCCAAGGG
ACTCTGGTCACTGTCTCTGCAGCCTCAACGAAGGGCCCATCTGTCTTTCCCCTGGCCCCCTCCTCC
AAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGT
GAATTCACTCACACATGCCCACCGTGCCCAGCACCTGAACTCCTGGGGGGACCGTCAGTCTTCCTC
TTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCCAGCC
CCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCC
AGCGACATCGCCGTGGAGTGGGAGAGCAATGGGCAGCCGGAGAACAACTACAAGACCACGCCTCCC
GTGCTGGACTCCGACGGCTCCTTCTTCCTCTACAGCAAGCTCACCGTGGACAAGAGCAGGTGGCAG
CAGGGGAACGTCTTCTCATGCTCCGTGATGCATGAGGCTCTGCACAACCACTACACGCAGAAGAGC
CTCTCCCTGTCTCCCGGGAAA
SEQ ID NO.: 14
EVQLQESGPDLVKPSQSLSLTCTVTGFSITSGYGWHWIRQFPGNKLEWMGYINYDGHNDYNPSLKS
RISITQDTSKNQFFLQLNSVTTEDTATYYCASSYDGLFAYWGQGTLVTVSAASTKGPSVFPLAPSS
KSTSGGTAALGCLVKDYFPEPVTVSWNSGALTSGVHTFPAVLQSSGLYSLSSVVTVPSSSLGTQTY
ICNVNHKPSNTKVDKKVEPKSCEFTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO.: 15
GACATTGTGATGACCCAGTCTCCATCCTCCCTGGCTGTGTCAATAGGACAGAAGGTCACTATGAAC
TGCAAGTCCAGTCAGAGCCTTTTAAATAGTAACTTTCAAAAGAACTTTTTGGCCTGGTACCAGCAG
AAACCAGGCCAGTCTCCTAAACTTCTGATATACTTTGCATCCACTCGGGAATCTAGTATCCCTGAT
CGCTTCATAGGCAGTGGATCTGGGACAGATTTCACTCTTACCATCAGCAGTGTGCAGGCTGAAGAC
CTGGCAGATTACTTCTGTCAGCAACATTATAGCACTCCGCTCACGTTCGGTGCTGGGACCAAGCTG
GAGCTGAAA
SEQ ID NO.: 16
DIVMTQSPSSLAVSIGQKVTMNCKSSQSLLNSNFQKNFLAWYQQKPGQSPKLLIYFASTRESSIPD
RFIGSGSGTDFTLTISSVQAEDLADYFCQQHYSTPLTFGAGTKLELK
SEQ ID NO.: 17
GAGGTTCAGCTGCAGCAGTCTGTAGCTGAGCTGGTGAGGCCTGGGGCTTCAGTGACGCTGTCCTGC
AAGGCTTCGGGCTACATATTTACTGACTATGAGATACACTGGGTGAAGCAGACTCCTGTGCATGGC
CTGGAATGGATTGGGGTTATTGATCCTGAAACTGGTAATACTGCCTTCAATCAGAAGTTCAAGGGC
AAGGCCACACTGACTGCAGACATATCCTCCAGCACAGCCTACATGGAACTCAGCAGTTTGACATCT
GAGGACTCTGCCGTCTATTACTGTATGGGTTATTCTGATTATTGGGGCCAAGGCACCACTCTCACA
GTCTCCTCA
SEQ ID NO.: 18
EVQLQQSVAELVRPGASVTLSCKASGYIFTDYEIHWVKQTPVHGLEWIGVIDPETGNTAFNQKFKG
KATLTADISSSTAYMELSSLTSEDSAVYYCMGYSDYWGQGTTLTVSS
SEQ ID NO.: 19
GATGTTTTGATGACCCAAACTCCACGCTCCCTGTCTGTCAGTCTTGGAGATCAAGCCTCCATCTCT
TGTAGATCGAGTCAGAGCCTTTTACATAGTAATGGAAACACCTATTTAGAATGGTATTTGCAGAAA
CCAGGCCAGCCTCCAAAGGTCCTGATCTACAAAGTTTCCAACCGATTTTCTGGGGTCCCAGACAGG
TTCAGTGGCAGTGGATCAGGGACAGATTTCACACTCAAGATCAGCGGAGTGGAGGCTGAGGATCTG
GGAGTTTATTACTGCTTTCAAGGTTCACATGTTCCTCTCACGTTCGGTGCTGGGACCAAGCTGGAG
CTGAAA
SEQ ID NO.: 20
DVLMTQTPRSLSVSLGDQASISCRSSQSLLHSNGNTYLEWYLQKPGQPPKVLIYKVSNRFSGVPDR
FSGSGSGTDFTLKISGVEAEDLGVYYCFQGSHVPLTFGAGTKLELK
SEQ ID NO.: 21
GAGATCCAGCTGCAGCAGTCTGGACCTGAGTTGGTGAAGCCTGGGGCTTCAGTGAAGATATCCTGT
AAGGCTTCTGGATACACCTTCACTGACAACTACATGAACTGGGTGAAGCAGAGCCATGGAAAGAGC
CTTGAGTGGATTGGAGATATTAATCCTTACTATGGTACTACTACCTACAACCAGAAGTTCAAGGGC
AAGGCCACATTGACTGTAGACAAGTCCTCCCGCACAGCCTACATGGAGCTCCGCGGCCTGACATCT
GAGGACTCTGCAGTCTATTACTGTGCAAGAGATGACTGGTTTGATTATTGGGGCCAAGGGACTCTG
GTCACTGTCTCTGCA
SEQ ID NO.: 22
EIQLQQSGPELVKPGASVKISCKASGYTFTDNYMNWVKQSHGKSLEWIGDINPYYGTTTYNQKFKG
KATLTVDKSSRTAYMELRGLTSEDSAVYYCARDDWFDYWGQGTLVTVSA
SEQ ID NO.: 23
GACATCGTTATGTCTCAGTCTCCATCTTCCATGTATGCATCTCTAGGAGAGAGAGTCACTATCACT
TGCAAGGCGAGTCAGGACATTCATAACTTTTTAAACTGGTTCCAGCAGAAACCAGGAAAATCTCCA
AAGACCCTGATCTTTCGTGCAAACAGATTGGTAGATGGGGTCCCATCAAGGTTCAGTGGCAGTGGA
TCTGGGCAAGATTATTCTCTCACCATCAGCAGCCTGGAGTTTGAAGATTTGGGAATTTATTCTTGT
CTACAGTATGATGAGATTCCGCTCACGTTCGGTGCTGGGACCAAGCTGGAGCTGAGA
SEQ ID NO.: 24
DIVMSQSPSSMYASLGERVTITCKASQDIHNFLNWFQQKPGKSPKTLIFRANRLVDGVPSRFSGSG
SGQDYSLTISSLEFEDLGIYSCLQYDEIPLTFGAGTKLELR
SEQ ID NO.: 25
GAGGTGCAGCTTCAGGAGTCAGGACCTGACCTGGTGAAACCTTCTCAGTCACTTTCACTCACCTGC
ACTGTCACTGGCTTCTCCATCACCAGTGGTTATGGCTGGCACTGGATCCGGCAGTTTCCAGGAAAC
AAACTGGAGTGGATGGGCTACATAAACTACGATGGTCACAATGACTACAACCCATCTCTCAAAAGT
CGAATCTCTATCACTCAAGACACATCCAAGAACCAGTTCTTCCTGCAGTTGAATTCTGTGACTACT
GAGGACACAGCCACATATTACTGTGCAAGCAGTTACGACGGCTTATTTGCTTACTGGGGCCAAGGG
ACTCTGGTCACTGTCTCTGCA
SEQ ID NO.: 26
EVQLQESGPDLVKPSQSLSLTCTVTGFSITSGYGWHWIRQFPGNKLEWMGYINYDGHNDYNPSLKS
RISITQDTSKNQFFLQLNSVTTEDTATYYCASSYDGLFAYWGQGTLVTVSA
SEQ.ID NO. 27
biotin-actgtactAACCCTGCGGCCGCTTTTTTTTTTTTTTTTTTTTV
SEQ.ID NO. 28
GGAATTCTAATACGACTCACTATAGGGAGACGAAGACAGTAGACAGG
SEQ.ID NO. 29
CGCGCCTGTCTACTGTCTTCGTCTCCCTATAGTGAGTCGTATTAGAATTC
SEQ.ID NO. 30
GGAATTCTAATACGACTCACTATAGGGAGAGCCTGCACCAACAGTTAACAGG
SEQ.ID NO. 31
CGCGCCTGTTAACTGTTGGTGCAGGCTCTCCCTATAGTGAGTCGTATTAGAATTC
SEQ.ID NO. 32
GGGAGACGAAGACAGTAGA
SEQ.ID NO. 33
GCCTGCACCAACAGTTAACA
SEQ.ID NO. 34
GGAATTCTAATACGACTCACTATAGGGA
SEQ.ID NO. 35
CGCGTCCCTATAGTGAGTCGTATTAGAATTC
SEQ.ID NO. 36
TTTTCCCAGTCACGACGTTGTAAAACGACGGCCAGTGAATTCTAATACGACTCACTATAGGGAGAT
GGAGAAAAAAATCACTGGACGCGTGGCGCGCCATTAATTAATGCGGCCGCTAGCTCGAGTGATAAT
AAGCGGATGAATGGCTGCAGGCATGCAAGCTTGGCGTAATCATGGTCATAGCTGTTTCCTGTGTGA
AATTGTTATCCGCTCACAATTCCACACAACATACGAGCCGGAAGCATAAAGTGTAAAGCCTGGGGT
GCCTAATGAGTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCA
AAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCAATGCTCACGCTGTAGGTAT
CTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTTGAAG
TGGTGGCCTAACTACGGCTACACTAGAAGGACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGCAATAAAC
CAGCCAGCCGGAAGGGCCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGAA
TAGTGTATGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAGGATCTTACCG
CTGTTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATATTATTGAAGCATTTATCAGGGTTATTGT
CTCATGAGCGGATACATATTTGAATGTATTTAGAAAAATAAACAAATAGGGGTTCCGCGCACATTT
CCCCGAAAAGTGCCACCTGACGTCTAAGAAACCATTATTATCATGACATTAACCTATAAAAATAGG
CGTATCACGAGGCCCTTTCGTCTCGCGCGTTTCGGTGATGACGGTGAAAACCTCTGACACATGCAG
CTCCCGGAGACGGTCACAGCTTGTCTGTAAGCGGATGCCGGGAGCAGACAAGCCCGTCAGGGCGCG
TCAGCGGGTGTTGGCGGGTGTCGGGGCTGGCTTAACTATGCGGCATCAGAGCAGATTGTACTGAGA
GTGCACCATATGCGGTGTGAAATACCGCACAGATGCGTAAGGAGAAAATACCGCATCAGGCGCCAT
TCGCCATTCAGGCTGCGCAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAGGG
SEQ.ID NO. 37
TTTTCCCAGTCACGACGTTGTAAAACGACGGCCAGTGAATTCAATTAACCCTCACTAAAGGGAGAC
TTGTTCCAAATGTGTTAGGcgCGCCGCATGCGTCGACGGATCCTGAGAACTTCAGGCTCCTGGGCA
ACGTGCTGGTTATTGTGCTGTCTCATCATTTTGGCAAAGAATTCACTCCTCAGGTGCAGGCTGCCT
ATCAGAAGGTGGTGGCTGGTGTGGCCAATGCCCTGGCTCACAAATACCACTGAGATCTTTTTCCCT
CTGCCAAAAATTATGGGGACATCATGAAGCCCCTTGAGCATCTGACTTCTGGCTAATAAAGGAAAT
TTATTTTCATTGCAAAAAAAAAAAGCGGCCGCTCTTCTATAGTGTCACCTAAATGGCCCAGCGGCC
GAGCTTGGCGTAATCATGGTCATAGCTGTTTCCTGTGTGAAATTGTTATCCGCTCACAATTCCACA
CAACATACGAGCCGGAAGCATAAAGTGTAAAGCCTGGGGTGCCTAATGAGTGAGCTAACTCACATT
AATTGCGTTGCGCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATACGGTTATC
CACAGAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAG
CTCCCTCGTGCGCTCTCCTGTTCCGACCCTGCCGCTTACCGGATACCTGTCCGCCTTTCTCCCTTC
GGGAAGCGTGGCGCTTTCTCAAAGCTCACGCTGTAGGTATCTCAGTTCGGTGTAGGTCGTTCGCTC
CAAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTGACGCTCAGTGGAACGA
AAACTCACGTTAAGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTC
GTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGT
GTTATCACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAGTTGCTC
TTGCCCGGCGTCAATACGGGATAATACCGCGCCACATAGCAGAACTTTAAAAGTGCTCATCATTGG
AAAACGTTCTTCGGGGCGAAAACTCTCAAGGATCTTACCGCTGTTGAGATCCAGTTCGATGTAACC
CACTCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAATG
TATTTAGAAAAATAAACAAATAGGGGTTCCGCGCACATTTCCCCGAAAAGTGCCACCTGACGTCTA
AGAAACCATTATTATCATGACATTAACCTATAAAAATAGGCGTATCACGAGGCCCTTTCGTCTCGC
GCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACC
GCACAGATGCGTAAGGAGAAAATACCGCATCAGGCGCCATTCGCCATTCAGGCTGCGCAACTGTTG
GGAAGGGCGATCGGTGCGGGCCTCTTCGCTATTACGCCAGCTGGCGAAAGGGGGATGTGCTGCAAG
GCGATTAAGTTGGGTAACGCCAGGG
SEQ.ID NO. 38
TGAAGGTCGGAGTCAACGGATTTGGT
SEQ.ID NO. 39
CATGTGGGCCATGAGGTCCACCAC
SEQ ID NO.: 40
GAGGGGCATCAATCACACCGAGAA
SEQ ID NO.: 41
CCCCACCGCCCACCCATTTAGG
SEQ ID NO.: 42
TGAAGGTCGGAGTCAACGGATTTGGT
SEQ ID NO.: 43
CATGTGGGCCATGAGGTCCACCAC
SEQ ID NO.: 44
GGCCTCCAGCCACGTAATT
SEQ ID NO.: 45
GGCGCTGCTGCCGCTCATC
SEQ ID NO.: 46
TCGCGCGTTTCGGTGATGACGGTGAAAACCTCTGACACATGCAGCTCCCGGAGACGGTCACAGCTT
GTCTGTAAGCGGATGCCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACCATATGCGGTGTGAAA
TACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTG
CCAAGCTTTTCCAAAAAACTACCGTTGTTATAGGTGTCTCTTGAACACCTATAACAACGGTAGTGG
ATCCCGCGTCCTTTCCACAAGATATATAAACCCAAGAAATCGAAATACTTTCAAGTTACGGTAAGC
ATATGATAGTCCATTTTAAAACATAATTTTAAAACTGCAAACTACCCAAGAAATTATTACTTTCTA
CGTCACGTATTTTGTACTAATATCTTTGTGTTTACAGTCAAATTAATTCTAATTATCTCTCTAACA
GCCTTGTATCGTATATGCAAATATGAAGGAATCATGGGAAATAGGCCCTCTTCCTGCCCGACCTTG
GCGCGCGCTCGGCGCGCGGTCACGCTCCGTCACGTGGTGCGTTTTGCCTGCGCGTCTTTCCACTGG
GGAATTCATGCTTCTCCTCCCTTTAGTGAGGGTAATTCTCTCTCTCTCCCTATAGTGAGTCGTATT
AATTCCTTCTCTTCTATAGTGTCACCTAAATCGTTGCAATTCGTAATCATGTCATAGCTGTTTCCT
GTGTGAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAAAAAACCACCGCTGGTAGCGGT
GGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGC
ACATTTCCCCGAAAAGTGCCACCTATTGGTGTGGAAAGTCCCCAGGCTCCCCAGCAGGCAGAAGTA
TGCAAAGCATGCATCTCAATTAGTCAGCAACCAGGTGTGGAAAGTCCCCAGGCTCCCCAGCAGGCA
GAAGTATGCAAAGCATGCATCTCAATTAGTCAGCAACCATAGTCCCGCCCCTAACTCCGCCCATCC
CGCCCCTAACTCCGCCCAGTTCCGCCCATTCTCCGCCCCATGGCTGACTAATTTTTTTTATTTATG
CAGAGGCCGAGGCCGCCTCGGCCTCTGAGCTATTCCAGAAGTAGTGAGGAGGCTTTTTTGGAGGCC
TAGGCTTTTGCAAAAAGCTAGCTTGCATGCCTGCAGGTCGGCCGCCACGACCGGTGCCGCCACCAT
CCCCTGACCCACGCCCCTGACCCCTCACAAGGAGACGACCTTCCATGACCGAGTACAAGCCCACGG
TGCGCCTCGCCACCCGCGACGACGTCCCCCGGGCCGTACGCACCCTCGCCGCCGCGTTCGCCGACT
ACCCCGCCACGCGCCACACCGTCGACCCGGACCGCCACATCGAGCGGGTCACCGAGCTGCAAGAAC
TCTTCCTCACGCGCGTCGGGCTCGACATCGGCAAGGTGTGGGTCGCGGACGACGGCGCCGCGGTGG
CGGTCTGGACCACGCCGGAGAGCGTCGAAGCGGGGGCGGTGTTCGCCGAGATCGGCCCGCGCATGG
CCGAGTTGAGCGGTTCCCGGCTGGCCGCGCAGCAACAGATGGAAGGCCTCCTGGCGCCGCACCGGC
CCAAGGAGCCCGCGTGGTTCCTGGCCACCGTCGGCGTCTCGCCCGACCACCAGGGCAAGGGTCTGG
GCAGCGCCGTCGTGCTCCCCGGAGTGGAGGCGGCCGAGCGCGCCGGGGTGCCCGCCTTCCTGGAGA
CCTCCGCGCCCCGCAACCTCCCCTTCTACGAGCGGCTCGGCTTCACCGTCACCGCCGACGTCGAGG
TGCCCGAAGGACCGCGCACCTGGTGCATGACCCGCAAGCCCGGTGCCTGACGCCCGCCCCACGACC
CGCAGCGCCCGACCGAAAGGAGCGCACGACCCCATGGCTCCGACCGAAGCCACCCGGGGCGGCCCC
GCCGACCCCGCACCCGCCCCCGAGGCCCACCGACTCTAGAGGATCATAATCAGCCATACCACATTT
GTAGAGGTTTTACTTGCTTTAAAAAACCTCCCACACCTCCCCCTGAACCTGAAACATAAAATGAAT
GCAATTGTTGTTGTTAACTTGTTTATTGCAGCTTATAATGGTTACAAATAAAGCAATAGCATCACA
AATTTCACAAATAAAGCATTTTTTTCACTGCAATCTAAGAAACCATTATTATCATGACATTAACCT
ATAAAAATAGGCGTATCACGAGGCCCTTTCGTC
SEQ ID NO.: 47
GTAAGCGGATCCATGGATGACGACGCGGCGCCC
SEQ ID NO.: 48
GTAAGCAAGCTTCTTCTCTTTCACCTGTGGATT
SEQ ID NO.: 49
GTACATTTATATTGGCTCATGTCCAATATGACCGCCATGTTGACATTGATTATTGACTAGTTATTA
ATAGTAATCAATTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGC
CCACTTGGCAGTACATCAAGTGTATCATATGCCAAGTCCGCCCCCTATTGACGTCAATGACGGTAA
ATGGCCCGCCTGGCATTATGCCCAGTACATGACCTTACGGGACTTTCCTACTTGGCAGTACATCTA
CGTATTAGTCATCGCTATTACCATGGTGATGCGGTTTTGGCAGTACACCAATGGGCGTGGATAGCG
GTTTGACTCACGGGGATTTCCAAGTCTCCACCCCATTGACGTCAATGGGAGTTTGTTTTGGCACCA
AAATCAACGGGACTTTCCAAAATGTCGTAATAACCCCGCCCCGTTGACGCAAATGGGCGGTAGGCG
TGTACGGTGGGAGGTCTATATAAGCAGAGCTCGTTTAGTGAACCGTCAGATCCTCACTCTCTTCCG
CATCGCTGTCTGCGAGGGCCAGCTGTTGGGCTCGCGGTTGAGGACAAACTCTTCGCGGTCTTTCCA
GTACTCTTGGATCGGAAACCCGTCGGCCTCCGAACGGTACTCCGCCACCGAGGGACCTGAGCCAGT
CCGCATCGACCGGATCGGAAAACCTCTCGAGAAAGGCGTCTAACCAGTCACAGTCGCAAGGTAGGC
TGAGCACCGTGGCGGGCGGCAGCGGGTGGCGGTCGGGGTTGTTTCTGGCGGAGGTGCTGCTGATGA
TGTAATTAAAGTAGGCGGTCTTGAGCCGGCGGATGGTCGAGGTGAGGTGTGGCAGGCTTGAGATCC
AGCTGTTGGGGTGAGTACTCCCTCTCAAAAGCGGGCATGACTTCTGCGCTAAGATTGTCAGTTTCC
AAAAACGAGGAGGATTTGATATTCACCTGGCCCGATCTGGCCATACACTTGAGTGACAATGACATC
CACTTTGCCTTTCTCTCCACAGGTGTCCACTCCCAGGTCCAAGTTTGCCGCCACCATGGAGACAGA
CACACTCCTGCTATGGGTACTGCTGCTCTGGGTTCCAGGTTCCACTGGCGCCGGATCAACTCACAC
ATGCCCACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGATGA
GCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGTTGGACTCCGA
CGGCTCCTTCTTCCTCTACAGCAAGCTCACCGTGGACAAGAGCAGGTGGCAGCAGGGGAACGTCTT
CTCATGCTCCGTGATGCATGAGGCTCTGCACAACCACTACACGCAGAAGAGCCTCTCCCTGTCTCC
CGGGAAAGCTAGCGGAGCCGGAAGCACAACCGAAAACCTGTATTTTCAGGGCGGATCCGAATTCAA
GCTTGATATCTGATCCCCCGACCTCGACCTCTGGCTAATAAAGGAAATTTATTTTCATTGCAATAG
TGTGTTGGAATTTTTTGTGTCTCTCACTCGGAAGGACATATGGGAGGGCAAATCATTTGGTCGAGA
TCCCTCGGAGATCTCTAGCTAGAGCCCCGCCGCCGGACGAACTAAACCTGACTACGGCATCTCTGC
CCCTTCTTCGCGGGGCAGTGCATGTAATCCCTTCAGTTGGTTGGTACAACTTGCCAACTGAACCCT
AAACGGGTAGCATATGCTTCCCGGGTAGTAGTATATACTATCCAGACTAACCCTAATTCAATAGCA
TATGTTACCCAACGGGAAGCATATGCTATCGAATTAGGGTTAGTAAAAGGGTCCTAAGGAACAGCG
ATGTAGGTGGGCGGGCCAAGATAGGGGCGCGATTGCTGCGATCTGGAGGACAAATTACACACACTT
GCGCCTGAGCGCCAAGCACAGGGTTGTTGGTCCTCATATTCACGAGGTCGCTGAGAGCACGGTGGG
CTAATGTTGCCATGGGTAGCATATACTACCCAAATATCTGGATAGCATATGCTATCCTAATCTATA
TCTGGGTAGCATAGGCTATCCTAATCTATATCTGGGTAGCATATGCTATCCTAATCTATATCTGGG
TAGTATATGCTATCCTAATTTATATCTGGGTAGCATAGGCTATCCTAATCTATATCTGGGTAGCAT
ATGCTATCCTAATCTATATCTGGGTAGTATATGCTATCCTAATCTGTATCCGGGTAGCATATGCTA
TCCTAATAGAGATTAGGGTAGTATATGCTATCCTAATTTATATCTGGGTAGCATATACTACCCAAA
TATCTGGATAGCATATGCTATCCTAATCTATATCTGGGTAGCATATGCTATCCTAATCTATATCTG
GGTAGCATAGGCTATCCTAATCTATATCTGGGTAGCATATGCTATCCTAATCTATATCTGGGTAGT
ATATGCTATCCTAATTTATATCTGGGTAGCATAGGCTATCCTAATCTATATCTGGGTAGCATATGC
TATCCTAATCTATATCTGGGTAGTATATGCTATCCTAATCTGTATCCGGGTAGCATATGCTATCCT
CACGATGATAAGCTGTCAAACATGAGAATTAATTCTTGAAGACGAAAGGGCCTCGTGATACGCCTA
TTTTTATAGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCATGAGACAA
TAACCCTGATAAATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATCTCAACAGCGGT
AAGATCCTTGAGAGTTTTCGCCCCGAAGAACGTTTTCCAATGATGAGCACTTTTAAAGTTCTGCTA
TGTGGCGCGGTATTATCCCGTGTTGACGCCGGGCAAGAGCAACTCGGTCGCCGCATACACTATTCT
CAGAATGACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTTGATCGT
TGGGAACCGGAGCTGAATGAAGCCATACCAAACGACGAGCGTGACACCACGATGCCTGCAGCAATG
GCAACAACGTTGCGCAAACTATTAACTGGCGAACTACTTACTCTAGCTTCCCGGCAACAATTAATA
GACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTT
GATAATCTCATGACCAAAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTCCGAAGGTAACT
GGCTTCAGCAGAGCGCAGATACCAAATACTGTCCTTCTAGTGTAGCCGTAGTTAGGCCACCACTTC
AAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGATAC
CTACAGCGTGAGCATTGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTGAGTGAGCT
GATACCGCTCGCCGCAGCCGAACGACCGAGCGCAGCGAGTCAGTGAGCGAGGAAGC
SEQ ID NO.: 50
GTAAGCAAGCTTAGGCCGCTGGGACAGCGGAGGTGC
SEQ ID NO.: 51
GTAAGCAAGCTTGGCAGCAGCGCCAGGTCCAGC
SEQ ID NO.: 52
GTAAGCAGCGCTGTGGCTGCACCATCTGTCTTC
SEQ ID NO.: 53
GTAAGCGCTAGCCTAACACTCTCCCCTGTTGAAGC
SEQ ID NO.: 54
GCTGTGGCTGCACCATCTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACAGTGGAAGGTGGATAAC
GCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTC
ACCCATCAGGGCCTGAGCTCGCCCGTCACAAAGAGCTTCAACAGGGGAGAGTGTTAG
SEQ ID NO.: 55
AVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO.: 56
CTTGAGCCGGCGGATGGTCGAGGTGAGGTGTGGCAGGCTTGAGATCCAGCTGTTGGGGTGAGTACT
CCCTCTCAAAAGCGGGCATTACTTCTGCGCTAAGATTGTCAGTTTCCAAAAACGAGGAGGATTTGA
TATTCACCTGGCCCGATCTGGCCATACACTTGAGTGACAATGACATCCACTTTGCCTTTCTCTCCA
CAGGTGTCCACTCCCAGGTCCAAGTTTAAACGGATCTCTAGCGAATTCATGAACTTTCTGCTGTCT
TGGGTGCATTGGAGCCTTGCCTTGCTGCTCTACCTCCACCATGCCAAGTGGTCCCAGGCTTGAGAC
GGAGCTTACAGCGCTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGTGT
TAGGGTACCGCGGCCGCTTCGAATGAGATCCCCCGACCTCGACCTCTGGCTAATAAAGGAAATTTA
TTTTCATTGCAATAGTGTGTTGGAATTTTTTGTGTCTCTCACTCGGAAGGACATATGGGAGGGCAA
ATCATTTGGTCGAGATCCCTCGGAGATCTCTAGCTAGAGCCCCGCCGCCGGACGAACTAAACCTGA
CTACGGCATCTCTGCCCCTTCTTCGCGGGGCAGTGCATGTAATCCCTTCAGTTGGTTGGTACAACT
TGCCAACTGGGCCCTGTTCCACATGTGACACGGGGGGGGACCAAACACAAAGGGGTTCTCTGACTG
TAGTTGACATCCTTATAAATGGATGTGCACATTTGCCAACACTGAGTGGCTTTCATCCTGGAGCAG
ACTTTGCAGTCTGTGGACTGCAACACAACATTGCCTTTATGTGTAACTCTTGGCTGAAGCTCTTAC
ACCAATGCTGGGGGACATGTACCTCCCAGGGGCCCAGGAAGACTACGGGAGGCTACACCAACGTCA
ATCAGAGGGGCCTGTGTAGCTACCGATAAGCGGACCCTCAAGAGGGCATTAGCAATAGTGTTTATA
AGGCCCCCTTGTTAACCCTAAACGGGTAGCATATGCTTCCCGGGTAGTAGTATATACTATCCAGAC
TAACCCTAATTCAATAGCATATGTTACCCAACGGGAAGCATATGCTATCGAATTAGGGTTAGTAAA
AGGGTCCTAAGGAACAGCGATATCTCCCACCCCATGAGCTGTCACGGTTTTATTTACATGGGGTCA
GGATTCCACGAGGGTAGTGAACCATTTTAGTCACAAGGGCAGTGGCTGAAGATCAAGGAGCGGGCA
GTGAACTCTCCTGAATCTTCGCCTGCTTCTTCATTCTCCTTCGTTTAGCTAATAGAATAACTGCTG
AGTTGTGAACAGTAAGGTGTATGTGAGGTGCTCGAAAACAAGGTTTCAGGTGACGCCCCCAGAATA
AAATTTGGACGGGGGGTTCAGTGGTGGCATTGTGCTATGACACCAATATAACCCTCACAAACCCCT
TGGGCAATAAATACTAGTGTAGGAATGAAACATTCTGAATATCTTTAACAATAGAAATCCATGGGG
TGGGGACAAGCCGTAAAGACTGGATGTCCATCTCACACGAATTTATGGCTATGGGCAACACATAAT
CCTAGTGCAATATGATACTGGGGTTATTAAGATGTGTCCCAGGCAGGGACCAAGACAGGTGAACCA
TGTTGTTACACTCTATTTGTAACAAGGGGAAAGAGAGTGGACGCCGACAGCAGCGGACTCCACTGG
TTGTCTCTAACACCCCCGAAAATTAAACGGGGCTCCACGCCAATGGGGCCCATAAACAAAGACAAG
TGGCCACTCTTTTTTTTGAAATTGTGGAGTGGGGGCACGCGTCAGCCCCCACACGCCGCCCTGCGG
TTTTGGACTGTAAAATAAGGGTGTAATAACTTGGCTGATTGTAACCCCGCTAACCACTGCGGTCAA
ACCACTTGCCCACAAAACCACTAATGGCACCCCGGGGAATACCTGCATAAGTAGGTGGGCGGGCCA
AGATAGGGGCGCGATTGCTGCGATCTGGAGGACAAATTACACACACTTGCGCCTGAGCGCCAAGCA
CAGGGTTGTTGGTCCTCATATTCACGAGGTCGCTGAGAGCACGGTGGGCTAATGTTGCCATGGGTA
GCATATACTACCCAAATATCTGGATAGCATATGCTATCCTAATCTATATCTGGGTAGCATAGGCTA
TCCTAATCTATATCTGGGTAGCATATGCTATCCTAATCTATATCTGGGTAGTATATGCTATCCTAA
TTTATATCTGGGTAGCATAGGCTATCCTAATCTATATCTGGGTAGCATATGCTATCCTAATCTATA
TCTGGGTAGTATATGCTATCCTAATCTGTATCCGGGTAGCATATGCTATCCTAATAGAGATTAGGG
TAGTATATGCTATCCTAATTTATATCTGGGTAGCATATACTACCCAAATATCTGGATAGCATATGC
TATCCTAATCTATATCTGGGTAGCATATGCTATCCTAATCTATATCTGGGTAGCATAGGCTATCCT
AATCTATATCTGGGTAGCATATGCTATCCTAATCTATATCTGGGTAGTATATGCTATCCTAATTTA
TATCTGGGTAGCATAGGCTATCCTAATCTATATCTGGGTAGCATATGCTATCCTAATCTATATCTG
GGTAGTATATGCTATCCTAATCTGTATCCGGGTAGCATATGCTATCCTCACGATGATAAGCTGTCA
AACATGAGAATTAATTCTTGAAGACGAAAGGGCCTCGTGATACGCCTATTTTTATAGGTTAATGTC
ATGATAATAATGGTTTCTTAGACGTCAGGTGGCACTTTTCGGGGAAATGTGCGCGGAACCCCTATT
TGTTTATTTTTCTAAATACATTCAAATATGTATCCGCTCATGAGACAATAACCCTGATAAATGCTT
CAATAATATTGAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCTTGAGAGTTTT
CGCCCCGAAGAACGTTTTCCAATGATGAGCACTTTTAAAGTTCTGCTATGTGGCGCGGTATTATCC
CGTGTTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGAGCTA
ACCGCTTTTTTGCACAACATGGGGGATCATGTAACTCGCCTTGATCGTTGGGAACCGGAGCTGAAT
GAAGCCATACCAAACGACGAGCGTGACACCACGATGCCTGCAGCAATGGCAACAACGTTGCGCAAA
CTATTAACTGGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGATAAATCTGGA
GCCGGTGAGCGTGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAGATTGAT
TTAAAACTTCATTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCATTGA
GAAAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTTCCTGCG
TTATCCCCTGATTCTGTGGATAACCGTATTACCGCCTTTGAGTGAGCTGATACCGCTCGCCGCAGC
CGAACGACCGAGCGCAGCGAGTCAGTGAGCGAGGAAGCGGAAGAGCGCCCAATACGCAAACCGCCT
CTCCCCGCGCGTTGGCCGATTCATTAATGCAGCTGGCACGACAGGTTTCCCGACTGGAAAGCGGGC
AGTGAGCGCAACGCAATTAATGTGAGTTAGCTCACTCATTAGGCACCCCAGGCTTTACACTTTATG
CTTCCGGCTCGTATGTTGTGTGGAATTGTGAGCGGATAACAATTTCACACAGGAAACAGCTATGAC
CATGATTACGCCAAGCTCTAGCTAGAGGTCGACCAATTCTCATGTTTGACAGCTTATCATCGCAGA
TCCGGGCAACGTTGTTGCATTGCTGCAGGCGCAGAACTGGTAGGTATGGCAGATCTATACATTGAA
TCAATATTGGCAATTAGCCATATTAGTCATTGGTTATATAGCATAAATCAATATTGGCTATTGGCC
ATTGCATACGTTGTATCTATATCATAATATGTACATTTATATTGGCTCATGTCCAATATGACCGCC
ATGTTGACATTGATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCATATGCCAAG
TCCGCCCCCTATTGACGTCAATGACGGTAAATGGCCCGCCTGGCATTATGCCCAGTACATGACCTT
ACGGGACTTTCCTACTTGGCAGTACATCTACGTATTAGTCATCGCTATTACCATGGTGATGCGGTT
TTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAATAACCC
CGCCCCGTTGACGCAAATGGGCGGTAGGCGTGTACGGTGGGAGGTCTATATAAGCAGAGCTCGTTT
AGTGAACCGTCAGATCCTCACTCTCTTCCGCATCGCTGTCTGCGAGGGCCAGCTGTTGGGCTCGCG
GTTGAGGACAAACTCTTCGCGGTCTTTCCAGTACTCTTGGATCGGAAACCCGTCGGCCTCCGAACG
GTACTCCGCCACCGAGGGACCTGAGCGAGTCCGCATCGACCGGATCGGAAAACCTCTCGAGAAAGG
CGTCTAACCAGTCACAGTCGCAAGGTAGGCTGAGCACCGTGGCGGGCGGCAGCGGGTGGCGGTCGG
GGTTGTTTCTGGCGGAGGTGCTGCTGATGATGTAATTAAAGTAGGCGGT
SEQ DI NO.: 57
ATGCCAAGTGGTCCCAGGCTGACATTGTGATGACCCAGTCTCC
SEQ ID NO.: 58
ATGCCAAGTGGTCCCAGGCTGATGTTTTGATGACCCAAACTCC
SEQ ID NO.: 59
ATGCCAAGTGGTCCCAGGCTGACATCGTTATGTCTCAGTCTCC
SEQ ID NO.: 60
GGGAAGATGAAGACAGATGGTGCAGCCACAGC
SEQ ID NO.: 61
GTAAGCGCTAGCGCCTCAACGAAGGGCCCATCTGTCTTTCCCCTGGCCCC
SEQ ID NO.: 62
GTAAGCGAATTCACAAGATTTGGGCTCAACTTTCTTG
SEQ ID NO.: 63
GCCTCCACCAAGGGCCCATCGGTCTTCCCCCTGGCACCCTCCTCCAAGAGCACCTCTGGGGGCACA
GCAGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACAAG
CCCAGCAACACCAAGGTGGACAAGAAAGTTGAGCCCAAATCTTGT
SEQ ID NO.: 64
ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSC
SEQ ID NO.: 65
CTTGAGCCGGCGGATGGTCGAGGTGAGGTGTGGCAGGCTTGAGATCCAGCTGTTGGGGTGAGTACT
CCCTCTCAAAAGCGGGCATTACTTCTGCGCTAAGATTGTCAGTTTCCAAAAACGAGGAGGATTTGA
TATTCACCTGGCCCGATCTGGCCATACACTTGAGTGACAATGACATCCACTTTGCCTTTCTCTCCA
CAGGTGTCCACTCCCAGGTCCAAGTTTGCCGCCACCATGGAGACAGACACACTCCTGCTATGGGTA
CTGCTGCTCTGGGTTCCAGGTTCCACTGGCGGAGACGGAGCTTACGGGCCCATCTGTCTTTCCCCT
GGCCCCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAAAGTTGA
GCCCAAATCTTGTGAATTCACTCACACATGCCCACCGTGCCCAGCACCTGAACTCCTGGGGGGACC
GTCAGTCTTCCTCTTCCCCCCAAAACCCAAGGACACCCTCATGATCTCCCGGACCCCTGAGGTCAC
ATGCGTGGTGGTGGACGTGAGCCACGAAGACCCTGAGGTCAAGTTCAACTGGTACGTGGACGGCGT
GGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCCCGAGAACCACAGGT
GTACACCCTGCCCCCATCCCGGGATGAGCTGACCAAGAACCAGGTCAGCCTGACCTGCCTGGTCAA
AGGCTTCTATCCCAGCGACATCGCCGTGGAGTGGGAGAGCAATGGGCAGCCGGAGAACAACTACAA
GACCACGCCTCCCGTGCTGGACTCCGACGGCTCCTTCTTCCTCTACAGCAAGCTCACCGTGGACAA
GAGCAGGTGGCAGCAGGGGAACGTCTTCTCATGCTCCGTGATGCATGAGGCTCTGCACAACCACTA
CACGCAGAAGAGCCTCTCCCTGTCTCCCGGGAAATGATCCCCCGACCTCGACCTCTGGCTAATAAA
GGAAATTTATTTTCATTGCAATAGTGTGTTGGAATTTTTTGTGTCTCTCACTCGGAAGGACATATG
GGAGGGCAAATCATTTGGTCGAGATCCCTCGGAGATCTCTAGCTAGAGCCCCGCCGCCGGACGAAC
TAAACCTGACTACGGCATCTCTGCCCCTTCTTCGCGGGGCAGTGCATGTAATCCCTTCAGTTGGTT
GGTACAACTTGCCAACTGAACCCTAAACGGGTAGCATATGCTTCCCGGGTAGTAGTATATACTATC
CAGACTAACCCTAATTCAATAGCATATGTTACCCAACGGGAAGCATATGCTATCGAATTAGGGTTA
GTAAAAGGGTCCTAAGGAACAGCGATGTAGGTGGGCGGGCCAAGATAGGGGCGCGATTGCTGCGAT
CTGGAGGACAAATTACACACACTTGCGCCTGAGCGCCAAGCACAGGGTTGTTGGTCCTCATATTCA
CGAGGTCGCTGAGAGCACGGTGGGCTAATGTTGCCATGGGTAGCATATACTACCCAAATATCTGGA
TAGCATATGCTATCCTAATCTATATCTGGGTAGCATAGGCTATCCTAATCTATATCTGGGTAGCAT
ATGCTATCCTAATCTATATCTGGGTAGTATATGCTATCCTAATTTATATCTGGGTAGCATAGGCTA
TCCTAATCTATATCTGGGTAGCATATGCTATCCTAATCTATATCTGGGTAGTATATGCTATCCTAA
TCTGTATCCGGGTAGCATATGCTATCCTAATAGAGATTAGGGTAGTATATGCTATCCTAATTTATA
TCTGGGTAGCATATACTACCCAAATATCTGGATAGCATATGCTATCCTAATCTATATCTGGGTAGC
ATATGCTATCCTAATCTATATCTGGGTAGCATAGGCTATCCTAATCTATATCTGGGTAGCATATGC
TATCCTAATCTATATCTGGGTAGTATATGCTATCCTAATTTATATCTGGGTAGCATAGGCTATCCT
AATCTATATCTGGGTAGCATATGCTATCCTAATCTATATCTGGGTAGTATATGCTATCCTAATCTG
TATCCGGGTAGCATATGCTATCCTCACGATGATAAGCTGTCAAACATGAGAATTAATTCTTGAAGA
CGAAAGGGCCTCGTGATACGCCTATTTTTATAGGTTAATGTCATGATAATAATGGTTTCTTAGACG
TCAGGTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAACGTTTTCCAATG
ATGAGCACTTTTAAAGTTCTGCTATGTGGCGCGGTATTATCCCGTGTTGACGCCGGGCAAGAGCAA
CTCGGTCGCCGCATACACTATTCTCAGAATGACTTGGTTGAGTACTCACCAGTCACAGAAAAGCAT
CTTACGGATGGCATGACAGTAAGAGAATTATGCAGTGCTGCCATAACCATGAGTGATAACACTGCG
GCCAACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACGAGCGT
GACACCACGATGCCTGCAGCAATGGCAACAACGTTGCGCAAACTATTAACTGGCGAACTACTTACT
CTAGCTTCCCGGCAACAATTAATAGACTGGATGGAGGCGGATAAAGTTGCAGGACCACTTCTGCGC
TCGGCCCTTCCGGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACACGACGGGGAGT
CAGGCAACTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTAATTTAAA
AGGATCTAGGTGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTCCTTCTAGTG
TAGCCGTAGTTAGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGCGA
ACGACCTACACCGAACTGAGATACCTACAGCGTGAGCATTGAGAAAGCGCCACGCTTCCCGAAGGG
AGAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGCGGCCTTTTTACGGTTC
CTGGCCTTTTGCTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGCGCAGCGAGTCA
GTGAGCGAGGAAGCGTACATTTATATTGGCTCATGTCCAATATGACCGCCATGTTGACATTGATTA
TTGACTAGTTATTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCCCATTGACGTCA
ATAATGACGTATGTTCCCATAGTAACGCCAATAGGGACTTTCCATTGACGTCAATGGGTGGAGTAT
TTACGGTAAACTGCCCACTTGGCAGTACATCAAGTGTATCATATGCCAAGTCCGCCCCCTATTGAC
GTCAATGACGGTAAATGGCCCGCCTGGCATTATGCCCAGTACATGACCTTACGGGACTTTCCTACT
TGGCAGTACATCTACGTATTAGTCATCGCTATTACCATGGTGATGCGGTTTTGGCAGTACACCAAT
GGGCGTGGATAGCGGTTTGACTCACGGGGATTTCCAAGTCTCCACCCCATTGACGTCAATGGGAGT
TTGTTTTGGCACCAAAATCAACGGGACTTTCCAAAATGTCGTAATAACCCCGCCCCGTTGACGCAA
ATGGGCGGTAGGCGTGTACGGTGGGAGGTCTATATAAGCAGAGCTCGTTTAGTGAACCGTCAGATC
CTCACTCTCTTCCGCATCGCTGTCTGCGAGGGCCAGCTGTTGGGCTCGCGGTTGAGGACAAACTCT
TCGCGGTCTTTCCAGTACTCTTGGATCGGAAACCCGTCGGCCTCCGAACGGTACTCCGCCACCGAG
GGACCTGAGCGAGTCCGCATCGACCGGATCGGAAAACCTCTCGAGAAAGGCGTCTAACCAGTCACA
GTCGCAAGGTAGGCTGAGCACCGTGGCGGGCGGCAGCGGGTGGCGGTCGGGGTTGTTTCTGGCGGA
GGTGCTGCTGATGATGTAATTAAAGTAGGCGGT
SEQ ID NO.: 66
GGGTTCCAGGTTCCACTGGCGAGGTTCAGCTGCAGCAGTCTGT
SEQ ID NO.: 67
GGGTTCCAGGTTCCACTGGCGAGGTGCAGCTTCAGGAGTCAGG
SEQ ID NO.: 68
GGGGCCAGGGGAAAGACAGATGGGCCCTTCGTTGAGGC
SEQ ID NO.: 69
MVLQTQVFISLLLWISGAYGDIVMTQSPDSLAVSLGERATINCKSSQSLLNSNFQKNFLAWYQQKP
GQPPKLLIYFASTRESSVPDRFSGSGSGTDFTLTISSLQAEDVAVYYCQQHYSTPLTFGQGTKLEI
KRTVAAPSVFIFPPSDEQLKSGTASVVCLLNNEYPREAKVQWKVDNALQSGNSQESVTEQDSKDST
YSLSSTLTLSKADYEKHKVYACEVTHQGLSSPVTKSFNRGEC
SEQ ID NO.: 70
MDWTWRILFLVAAATGTHAEVQLVQSGAEVKKPGASVKVSCKASGYIFTDYEIHWVRQAPGQGLEW
MGVIDPETGNTAFNQKFKGRVTITADTSTSTAYMELSSLTSEDTAVYYCMGYSDYWGQGTLVTVSS
ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPGK
SEQ ID No: 71
DIVMTQSPDSLAVSLGERATINCKSSQSLLNSNFQKNFLAWYQQKPGQPPKLLIYFASTRESSVPD
RFSGSGSGTDFTLTISSLQAEDVAVYYCQQHYSTPLTFGQGTKLEIK
SEQ ID NO.: 72
EVQLVQSGAEVKKPGASVKVSCKASGYIFTDYEIHWVRQAPGQGLEWMGVIDPETGNTAFNQKFKG
RVTITADTSTSTAYMELSSLTSEDTAVYYCMGYSDYWGQGTLVTVSS
SEQ ID NO.: 73
MVLQTQVFISLLLWISGAYGDIVMTQSPSSLSASVGDRVTITCKASQDIHNFLNWFQQKPGKAPKT
LIFRANRLVDGVPSRFSGSGSGTDYTLTISSLQPEDFATYSCLQYDEIPLTFGQGTKLEIKRTVAA
PSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO.: 74
MDWTWRILFLVAAATGTHAEVQLQESGPGLVKPSQTLSLTCTVSGFSITSGYGWHWIRQHPGKGLE
WIGYINYDGHNDYNPSLKSRVTISQDTSKNQFSLKLSSVTAADTAVYYCASSYDGLFAYWGQGTLV
TVSSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLSL
SPGK
SEQ ID No.: 75
DIVMTQSPSSLSASVGDRVTITCKASQDIHNFLNWFQQKPGKAPKTLIFRANRLVDGVPSRFSGSG
SGTDYTLTISSLQPEDFATYSCLQYDEIPLTFGQGTKLEIK
SEQ ID NO.: 76
EVQLQESGPGLVKPSQTLSLTCTVSGFSITSGYGWHWIRQHPGKGLEWIGYINYDGHNDYNPSLKS
RVTISQDTSKNQFSLKLSSVTAADTAVYYCASSYDGLFAYWGQGTLVTVS

### REFERENCES

Jemal A, Murray T, Ward E, Samuels A, Tiwari R C, Ghafoor A, Feuer E J and Thun M J. Cancer statistics, 2005. CA Cancer J Clin 2005; 55:10-30.
Menon U, Skates S J, Lewis S, Rosenthal A N, Rufford B, Sibley K, Macdonald N, Dawnay A, Jeyarajah A, Bast R C Jr, Oram D and Jacobs I J. Prospective study using the risk of ovarian cancer algorithm to screen for ovarian cancer. J Clin Oncol. 2005 Nov. 1; 23(31):7919-26.
Bonome T, Lee J Y, Park D C, Radonovich M, Pise-Masison C, Brady J, Gardner G J, Hao K, Wong W H, Barrett J C, Lu K H, Sood A K, Gershenson D M, Mok S C and Birrer M J. Expression profiling of serous low malignancy potential, low grade, and high grade tumors of the ovary. Cancer Res 2005; 65:10602-10612.
Chambers, A. and Vanderhyden, B. Ovarian Cancer Biomarkers in Urine. Clin Cancer Res 2006; 12(2):323-327.
Berek et al. Cancer Medicine. 5th ed. London: B. C. Decker, Inc.; 2000. p. 1687-1720.
Bristow R. E. Surgical standards in the management of ovarian cancer. Curr Opin Oncol 2000; 12:474-480.
Brown E, Stewart M, Rye T, Al-Nafussi A, Williams A R, Bradburn M, Smyth J and Gabra H. Carcinosarcoma of the ovary: 19 years of prospective data from a single center. Cancer 2004; 100:2148-2153.
Shih, L-M and Kurman, R J. Molecular Pathogenesis of Ovarian Borderline Tumors: New Insights and Old Challenges. Clin Cancer Res 2005; 11(20):7273-7279.
Seidman J D, Russell P, Kurman R J. Surface epithelial tumors of the ovary. In: Kurman R J, editor. Blaustein's pathologyo f the female genital tract. 5th ed. New York: Springer-Verlag; 2002. pp. 791-904.
Mor G, Visintin I, Lai Y, Zhao H, Schwartz P, Rutherford T, Yue L, Bray-Ward P and Ward D C Serum protein markers for early detection of ovarian cancer. PNAS 2005; 102:7677-7682.
Kozak K R, Amneus M W, Pusey S M, Su F, Luong M N, Luong S A, Reddy S T and Farias-Eisner R. Identification of biomarkers for ovarian cancer using strong anion-exchange ProteinChips: potential use in diagnosis and prognosis. PNAS 2003; 100:12343-12348.
McIntosh M W, Drescher C, Karlan B, Scholler N, Urban N, Hellstrom K E and Hellstrom I. Gynecol Oncol. 2004 October; 95(1):9-15.
Woolas R P, Xu F J, Jacobs I J, Yu Y H, Daly L, Berchuck A, Soper J T, Clarke-Pearson D L, Oram D H and Bast R C Jr. Elevation of multiple serum markers in patients with stage I ovarian cancer. J Natl Cancer Inst. 1993 Nov. 3; 85(21):1748-51.
Schorge J O, Drake R D, Lee H, Skates S J, Rajanbabu R, Miller D S, Kim J H, Cramer D W, Berkowitz R S and Mok S C. Osteopontin as an adjunct to CA125 in detecting recurrent ovarian cancer. Clin. Cancer Res. 10; 3474-3478.
Gorelik E, Landsittel D P, Marrangoni A M, Modugno F, Velikokhatnaya L, Winans M T, Bigbee W L, Herberman R B and Lokshin A E. Multiplexed Immunobead-Based Cytokine Profiling for Early Detection of Ovarian Cancer Cancer Epidemiol Biomarkers Prey. 2005 April; 14(4):981-7. Mack et al., 2003. U.S. Patent Application 20030124579.
Monahan et al., 2003. U.S. Patent Application 20030087250.
Wonsey et al., 2006. U.S. Patent Application 20060014686.
Santin A. D., 2006. U.S. Patent Application 20060078941.
Santin A. D., 2006. U.S. Patent Application 20060084594.
Jazaeri et al., 2005. U.S. Patent Application 20050095592.
Monahan et al., 2005. U.S. Patent Application 20050214831.
Gordon et al., 2003. U.S. Patent Application 20030219760.
Mor et al., 2005. U.S. Patent Application 20050214826.
Jhaveri et al., 2006. Antisense oligonucleotides targeting folate receptor alpha, and use thereof. U.S. Pat. No. 7,030,236.
Agrawal N, Dasaradhi P V, Mohmmed A, Malhotra P, Bhatnagar R K and Mukherjee S K. RNA interference: biology, mechanism, and applications. Microbiol. Mol Biol Rev. 2003 December; 67(4):657-85.
Brummelkamp T R, Bernards R and Agami R. A system for stable expression of short interfering RNAs in mammalian cells. Science 2002. 296: 550-553.
Elbashir S M, Harborth J, Lendeckel W, Yalcin A, Weber K, and Tuschl T. Duplexes of 21-nucleotide RNAs mediate RNA interference in cultured mammalian cells. Nature 2001. 411: 494-498.
Hannon G J. RNA interference. Nature 2002. 418: 244-251.
Leamon C P and Low P S. Folate-mediated targeting: from diagnostics to drug and gene delivery. Drug Discov Today 2001. 6: 44-51.
Mosmann T. Rapid colorimetric assay for cellular growth and survival: application to proliferation and cytotoxicity assays. J Immunol Methods. 1983. 65: 55-63.
Provencher D M, Lounis H, Champoux L, Tetrault M, Manderson E N, Wang J C, Eydoux P, Savoie R, Tonin P N and Mes-Masson A M. Characterization of four novel epithelial ovarian cancer cell lines. In Vitro Cell Dev Biol Anim 2000. 36: 357-361.
Samouelian V, Maugard C M, Jolicoeur M, Bertrand R, Arcand S L Tonin P N,
Provencher D M, and Mes-Masson A M. Chemosensitivity and radiosensitivity profiles of four new human epithelial ovarian cancer cell lines exhibiting genetic alterations in BRCA2, TGFbeta-RII, KRAS2, TP53 and/or CDNK2A. Cancer Chemother Pharmacol 2004. 54: 497-504.
Cannistra S A and McGuire W P. Progress in the management of gynecologic cancer. J. Clin. Oncol. 2007; 25(20): 2865-2866.
Oei A L, Sweep F C, Thomas C M, Boerman O C, Massuger L F. The use of monoclonal antibodies for the treatment of epithelial ovarian cancer. Int. J. Oncol. 2008; 32(6): 1145-1157.
Nicodemus C F and Berek J S. Monoclonal antibody therapy of ovarian cancer. Expert Rev. Anticancer Ther. 2005; 5(1): 87-96.
Burger R A. Experience with bevacizumab in the management of epithelial ovarian cancer. J. Clin. Oncol. 2007; 25(20): 2902-2908.
Simon I, Zhuo S, Corral L, Diamandis E P, Sarno M J, Wolfert R L, Kim N R. B7-H4 is a novel membrane-bound protein and a candidate serum and tissue biomerker for ovarian cancer. Cancer Res. 2006; 66(3): 1570-1575.
Ebel W, Routhier E L, Foley B, Jacob S, McDonough J M, Patel R K, Turchin H A, Chao Q, Kline J B, Old L J, Phillips M D, Nicolaides N C, Sass P M, Grasso L. Preclinical evaluation of MORab-003, a humanized monoclonal antibody antagonizing folate receptor-alpha. Cancer Immun. 2007; 7: 6-13.
Van den Eynde B J, Gaugler B, Probst-Kepper M, Michaux L, Devuyst O, Lorge F, Weynants P, Boon T. A new antigen recognized by cytotoxic T lymphocytes on a human kidney tumor results from reverse strand transcription. J. Exp. Med. 1999; 190(12): 1793-1799.
Van den Eynde: PCT/US99/10424.
Sooknanan R, Tremblay G B, Filion M. Polynucleotides and polypeptide sequences involved in cancer. 2007; PCT/CA2007/001134.
Schumacher J, Anthoni H, Dandouh F, Konig I R, Hillmer A M, Kluck N, Manthey M, Plume E, Warnke A, Remschmidt H, Hülsmann J, Cichon S, Lindgren C M, Propping P, Zucchelli M, Ziegler A, Peyrard-Janvid M, Schulte-Körne G, Nöthen M M, Kere J. Strong genetic evidence of DCDC2 as a susceptibility gene for dyslexia. Am. J. Hum. Genet. 2006; 78: 52-62.
Cope N, Harold D, Hill G, Moskvina V, Stevenson J, Holmans P, Owen M J, O'Donovan M C, Williams J. Strong evidence that KIAA0319 on chromosome 6p is a susceptibility gene for developmental dyslexia. Am. J. Hum. Genet. 2005; 76: 581-591.
Benoit M H, Hudson T J, Maire G, Squire J A, Arcand S L, Provencher D, Mes-Masson A M, Tonin P N. Global analysis of chromosome X gene expression in primary cultures of normal ovarian surface epithelial cells and epithelial ovarian cancer cell lines. Int. J. Oncol. 2007; 30(1): 5-17.
Cody N A, Zietarska M, Filali-Mouhim A, Provencher D M, Mes-Masson A M, Tonin P N. Influence of monolayer, spheroid, and tumor growth conditions on chromosome 3 gene expression in tumorigenic epithelial ovarian cancer cell lines. BMC Med. Genomics 2008; 1(1):34.
Buechler J, Valkirs G, Gray J. Polyvalent display libraries. 2000; U.S. Pat. No. 6,057,098.
Durocher Y, Kamen A, Perret S, Pham P L. Enhanced production of recombinant proteins by transient transfection of suspension-growing mammalian cells. 2002; Canadian patent application No. CA 2446185.
Durocher Y. Expression vectors for enhanced transient gene expression and mammalian cells expressing them. 2004; U.S. patent application No. 60/662,392.

What is claimed is:

1. A method of treating melanoma or renal cancer comprising administering an antibody or antigen binding fragment which specifically binds to a polypeptide having an amino acid sequence at least 80% identical to SEQ ID NO.:2 (KAAG1) to a subject in need.

2. The method of claim 1, wherein the polypeptide has a sequence at least 90% identical to SEQ ID NO.:1.

3. The method of claim 2, wherein the polypeptide has a sequence at least 95% identical to SEQ ID NO.:1.

4. The method of claim 3, wherein the polypeptide has a sequence identical to SEQ ID NO.:1.

5. The method of claim 1, wherein the antibody or antigen binding fragment is capable of impairing an activity of the polypeptide in renal cancer cells or in melanoma cells.

6. The method of claim 1, wherein the antibody or antigen binding fragment is capable of impairing an activity of the polypeptide in ovarian cancer cells.

7. The method of claim 5, wherein the antibody or antigen binding fragment is capable of impairing an activity of the polypeptide in ovarian cancer cells.

8. The method of claim 7, wherein the activity is ovarian cancer tumorigenesis.

9. The method of claim 1, wherein the cancer is malignant.

10. The method of claim 1, wherein the cancer is characterized as a late-stage.

11. The method of claim 1, wherein said antibody is a polyclonal antibody.

12. The method of claim 1, wherein the antibody is a monoclonal antibody.

13. The method of claim 1, wherein the antibody is a chimeric antibody.

14. The method of claim 1, wherein the antibody is a humanized antibody.

15. The method of claim 1, wherein the antibody is a human antibody.

16. The method of claim 1, wherein the antigen binding fragment is a FV, a Fab, a Fab′ or a (Fab′)₂.

17. The method of claim 1, wherein the antibody is conjugated with a chemotherapeutic agent.

18. The method of claim 1, wherein the subject in need has or is suspected of having renal cancer.

19. The method of claim 1, wherein the subject in need has or is suspected of having a melanoma.
